(19) United States
(12) Patent Application Publication
(10) Pub. No.: US 2023/0002408 A1
(43) Pub. Date: Jan. 5, 2023

(54) HETEROARYLDIHYDROPYRIMIDINE DERIVATIVES AND METHODS OF TREATING HEPATITIS B INFECTIONS

(71) Applicant: JANSSEN PHARMACEUTICA NV, Beerse (BE)

(72) Inventors: Gang DENG, Shanghai (CN); Zhanling CHENG, Shanghai (CN); Zhiguo LIU, Shanghai (CN); Chao LIANG, Shanghai (CN); Jianping WU, Shanghai (CN); Linglong KONG, Shanghai (CN); Xiangjun DENG, Shanghai (CN); Yimin JIANG, Shanghai (CN); Yanping XU, Shanghai (CN)

(21) Appl. No.: 17/755,896

(22) PCT Filed: Dec. 18, 2020
(86) PCT No.: PCT/CN2020/137480

§ 371 Date: May 11, 2022

(30) Foreign Application Priority Data
Dec. 20, 2019 (CN) PCT/CN2019/127038

(51) Int. Cl. C07D 498/04

(52) U.S. Cl. CPC C07D 498/04

(57) ABSTRACT

Provided herein are compounds useful for the treatment of HBV infection in a subject in need thereof, pharmaceutical compositions thereof, and methods of inhibiting, suppressing, or preventing HBV infection in the subject.

### BACKGROUND

Chronic hepatitis B virus (HBV) infection is a significant global health problem, affecting over 5% of the world population (over 350 million people worldwide and 1.25 million individuals in the U.S.).

Despite the availability of a prophylactic HBV vaccine, the burden of chronic HBV infection continues to be a significant unmet worldwide medical problem, due to suboptimal treatment options and sustained rates of new infections in most parts of the developing world. Current treatments do not provide a cure and are limited to only two classes of agents (interferon alpha and nucleoside analogues/inhibitors of the viral polymerase); drug resistance, low efficacy, and tolerability issues limit their impact. The low cure rates of HBV are attributed at least in part to the fact that complete suppression of virus production is difficult to achieve with a single antiviral agent. However, persistent suppression of HBV DNA slows liver disease progression and helps to prevent hepatocellular carcinoma. Current therapy goals for HBV-infected patients are directed to reducing serum HBV DNA to low or undetectable levels, and to ultimately reducing or preventing the development of cirrhosis and hepatocellular carcinoma.

The HBV capsid protein plays essential functions during the viral life cycle. HBV capsid/core proteins form metastable viral particles or protein shells that protect the viral genome during intercellular passage, and also play a central role in viral replication processes, including genome encapsidation, genome replication, and virion morphogenesis and egress. Capsid structures also respond to environmental cues to allow un-coating after viral entry. Consistently, the appropriate timing of capsid assembly and dis-assembly, the appropriate capsid stability and the function of core protein have been found to be critical for viral infectivity.

There is a need in the art for therapeutic agents that can increase the suppression of virus production and that can treat, ameliorate, or prevent HBV infection. Administration of such therapeutic agents to an HBV infected patient, either as monotherapy or in combination with other HBV treatments or ancillary treatments, will lead to significantly reduced virus burden, improved prognosis, diminished progression of the disease and enhanced seroconversion rates.

Background art on heteroaryldihydropyrimidines for use in the treatment of HBV includes WO 2015/132276, WO2013/102655, WO01/68641 and WO99/54326.

### SUMMARY

Provided herein are compounds useful for the treatment of HBV infection in a subject in need thereof. Thus, in an aspect, provided herein is a compound of Formula (I)

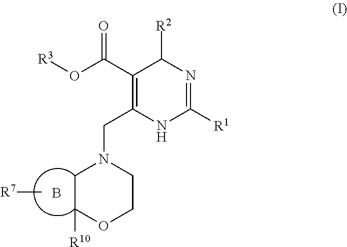

including the deuterated isomers, stereoisomers or tautomeric forms thereof, or a pharmaceutically acceptable salt thereof, wherein:

R¹ is selected from aryl or heteroaryl, each optionally substituted with one or more substituents selected from halogen, and C₁-C₆alkyl;

R² is selected from aryl or heteroaryl, each substituted with R⁴, R⁵, and R⁶;

R³ is C₁-C₄alkyl;

R⁴, R⁵, and R⁶ independently are selected from the group consisting of H, —OH, —CN, C₁-C₄alkyl, and halogen;

R¹⁰ is selected from the group consisting of H, and C₁-C₄alkyl;

Ring B is selected from the group consisting of 3-8 membered saturated or unsaturated rings, each of the rings optionally comprising 1, 2 or 3 heteroatoms selected from O, N, and S, and each of the rings optionally substituted with one or more substituents independently selected from the group consisting of halogen, ═O, —OH, —CN, C₁-C₄alkyl, C₁-C₄alkyloxy, and hydroxy C₁-C₄alkyl, wherein each C₁-C₄alkyl is optionally substituted with halogen;

R⁷ is selected from the group consisting of —SO₂—R⁸, —SO₂-Q-R⁸, —OC(═O)C₁-C₆alkyl, —C(═O)OC₁-C₆alkyl-COOH, —C(═O)NHC₁-C₆alkyl-COOH, —C(═O)O-Q-COOH, —C(═O)NH-Q-COOH, —C(═O)C₁-C₆alkyl, —C(═O)C₁-C₆alkyl-R⁸, —NHC₁-C₆alkyl-R⁸, —C₁-C₆alkyl, —C₁-C₆alkyl-R⁸, —C₁-C₆alkoxyC₁-C₆alkyl-R⁸, —(CH₂)ₚ—R⁸, —(CH₂)ₚ—C(R¹¹R¹²)—R⁸, and (CH₂)ₚ-Q-R⁸;

R⁸ is selected from the group consisting of —C₁-C₆alkyl, —C₁-C₆alkyl-COOH, —COOH, and carboxylic acid bioisosteres;

R¹¹ and R¹² together with the carbon atom to which they are attached form a 3-8 membered saturated ring optionally substituted with R⁹, the 3-8 membered saturated ring optionally containing a heteroatom, the heteroatom being N or 0;

Q is selected from the group consisting of aryl, heteroaryl, and a 3-8 membered saturated ring, each optionally substituted with R⁹, the 3-8 membered saturated ring optionally containing a heteroatom, the heteroatom being N or O;

R⁹ is selected from the group consisting of H, —C₁-C₆alkyl, —C₁-C₆alkoxyC₁-C₆alkyl and —C₁-C₆alkylcarbonyl;

p is an integer of 0, 1, 2, 3, or 4;

or a pharmaceutically acceptable salt or a solvate thereof.

In another aspect, provided herein is a pharmaceutical composition comprising at least one compound of Formula I, or a pharmaceutically acceptable salt thereof, together with a pharmaceutically acceptable carrier.

In another aspect, provided herein is a pharmaceutical composition comprising at least one disclosed compound, together with a pharmaceutically acceptable carrier.

In another aspect, provided herein is a method of treating an HBV infection or of an HBV-induced disease in an individual in need thereof, comprising administering to the individual a therapeutically effective amount of a compound of Formula I or a pharmaceutically acceptable salt thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula I, or a pharmaceutically acceptable salt thereof.

In an embodiment, any of the methods provided herein can further comprising administering to the individual at least one additional therapeutic agent selected from the group consisting of HBV inhibitors as herein further defined.

### DETAILED DESCRIPTION

Provided herein are compounds, e.g., the compounds of I, or pharmaceutically acceptable salts thereof, that are useful in the treatment and prevention of HBV infection in subject.

Without being bound to any particular mechanism of action, these compounds are believed to modulate or disrupt HBV assembly and other HBV core protein functions necessary for HBV replication or the generation of infectious particles and/or may disrupt HBV capsid assembly leading to empty capsids with greatly reduced infectivity or replication capacity. In other words, the compounds provided herein may act as capsid assembly modulators.

The compounds provided herein have potent antiviral activity, exhibit favorable metabolic properties, tissue distribution, safety and pharmaceutical profiles, and are suitable for use in humans. Disclosed compounds may modulate (e.g., accelerate, delay, inhibit, disrupt or reduce) normal viral capsid assembly or disassembly, bind capsid or alter metabolism of cellular polyproteins and precursors. The modulation may occur when the capsid protein is mature, or during viral infectivity. Disclosed compounds can be used in methods of modulating the activity or properties of HBV cccDNA, or the generation or release of HBV RNA particles from within an infected cell.

In one embodiment, the compounds described herein are suitable for monotherapy and are effective against natural or native HBV strains and against HBV strains resistant to currently known drugs. In another embodiment, the compounds described herein are suitable for use in combination therapy.

### Definitions

Listed below are definitions of various terms used to describe this invention. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include”, “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

As used herein, the term “capsid assembly modulator” refers to a compound that disrupts or accelerates or inhibits or hinders or delays or reduces or modifies normal capsid assembly (e.g., during maturation) or normal capsid disassembly (e.g., during infectivity) or perturbs capsid stability, thereby inducing aberrant capsid morphology and function. In one embodiment, a capsid assembly modulator accelerates capsid assembly or disassembly, thereby inducing aberrant capsid morphology. In another embodiment, a capsid assembly modulator interacts (e.g. binds at an active site, binds at an allosteric site, modifies or hinders folding and the like) with the major capsid assembly protein (CA), thereby disrupting capsid assembly or disassembly. In yet another embodiment, a capsid assembly modulator causes a perturbation in structure or function of CA (e.g., ability of CA to assemble, disassemble, bind to a substrate, fold into a suitable conformation, or the like), which attenuates viral infectivity or is lethal to the virus.

As used herein, the term “treatment” or “treating” is defined as the application or administration of a therapeutic agent, i.e., a disclosed compound (alone or in combination with another pharmaceutical agent), to a patient, or application or administration of a therapeutic agent to an isolated tissue or cell line from a patient (e.g., for diagnosis or ex vivo applications), who has an HBV infection, a symptom of HBV infection or the potential to develop an HBV infection, with the purpose to cure, heal, alleviate, relieve, alter, remedy, ameliorate, improve or affect the HBV infection, the symptoms of HBV infection, or the potential to develop an HBV infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “alkyl,” by itself or as part of another substituent means, unless otherwise stated, a straight or branched chain hydrocarbon having the number of carbon atoms designated (i.e., C₁-C₃alkyl means an alkyl having one to three carbon atoms, C₁-C₄alkyl means an alkyl having one to four carbon) and includes straight and branched chains. Examples include methyl, ethyl, propyl, isopropyl, butyl, isobutyl, tert-butyl. Embodiments of alkyl generally include, but are not limited to, C₁-C₁₀ alkyl, such as C₁-C₆ alkyl, such as C₁-C₄ alkyl.

As used herein, the term “alkenyl,” by itself or as part of another substituent means, unless otherwise stated, a linear or branched chain of hydrocarbons comprising at least one carbon to carbon double bond, having the number of carbon atoms designated (i.e., C₂-C₄ alkenyl or C₂₋₄alkenyl means an alkenyl having two to four to eight carbon atoms, C₄-C₈ alkenyl or C₄₋₈alkenyl means an alkenyl having four carbon atoms. Embodiments of alkenyl generally include, but are not limited to, C₂-C₆ alkenyl, such as C₂-C₄ alkenyl, such as C₂-C₃ alkenyl.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “3-8 membered saturated ring” refers to a mono cyclic non-aromatic saturated radical, wherein each of the atoms forming the ring (i.e., skeletal atoms) is a carbon atom, unless such ring contains one or more heteroatoms if so further defined. 3-8 Membered saturated rings include groups having 3 to 8 ring atoms. Monocyclic 3-8 membered saturated rings include, but are not limited to, cyclopropyl, cyclobutyl, cyclopentyl, cyclohexyl, cycloheptyl, cyclooctyl.

As used herein, a 3-8 membered saturated ring may optionally contain 1, 2 or 3 heteroatoms selected from O, N, and S, and each of the rings optionally substituted with one or more substituents.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two, or three rings), wherein such rings may be attached together in a pendent manner, such as a biphenyl, or may be fused, such as naphthalene. Examples of aryl groups include phenyl, anthracyl, and naphthyl. Preferred examples are phenyl (e.g., C₆-aryl) and biphenyl (e.g., C₁₂-aryl). In some embodiments, aryl groups have from six to sixteen carbon atoms. In some embodiments, aryl groups have from six to twelve carbon atoms (e.g., C₆-C₁₂-aryl). In some embodiments, aryl groups have six carbon atoms (e.g., C₆-aryl).

As used herein, the term “heteroaryl” or “heteroaromatic” refers to a heterocycle having aromatic character. Heteroaryl substituents may be defined by the number of carbon atoms, e.g., C₁-C₉-heteroaryl indicates the number of carbon atoms contained in the heteroaryl group without including the number of heteroatoms. For example, a C₁-C₉-heteroaryl will include an additional one to four heteroatoms. A polycyclic heteroaryl may include one or more rings that are partially saturated. Non-limiting examples of heteroaryls include pyridyl, pyrazinyl, pyrimidinyl (including, e.g., 2- and 4-pyrimidinyl), pyridazinyl, thienyl, furyl, pyrrolyl (including, e.g., 2-pyrrolyl), imidazolyl, thiazolyl, oxazolyl, pyrazolyl (including, e.g., 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Non-limiting examples of polycyclic heterocycles and heteroaryls include indolyl (including, e.g., 3-, 4-, 5-, 6- and 7-indolyl), indolinyl, quinolyl, tetrahydroquinolyl, isoquinolyl (including, e.g., 1- and 5-isoquinolyl), 1,2,3,4-tetrahydroisoquinolyl, cinnolinyl, quinoxalinyl (including, e.g., 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g., 3-, 4-, 5-, 6- and 7-benzofuryl), 2,3-dihydrobenzofuryl, 1,2-benzisoxazolyl, benzothienyl (including, e.g., 3-, 4-, 5-, 6-, and 7-benzothienyl), benzoxazolyl, benzothiazolyl (including, e.g., 2-benzothiazolyl and 5-benzothiazolyl), purinyl, benzimidazolyl (including, e.g., 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group.

As used herein, the terminology “selected from . . . ” (e.g., “R⁴ is selected from A, B and C”) is understood to be equivalent to the terminology “selected from the group consisting of . . . ” (e.g., “R⁴ is selected from the group consisting of A, B and C”).

One embodiment relates to a compound of Formula (I) as defined herein wherein the carboxylic acid bioisosteres are —S(═O)₂(OH), —P(═O)(OH)₂, —C(═O)NHOH, C(═O)NHCN, 1,2,4-oxadiazol-5(4H)-one, or 3-hydroxy-4-methylcyclobut-3-ene-1,2-dione. This refers to the following structures:

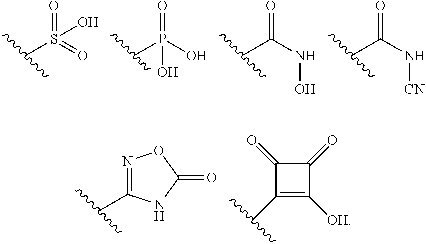 [SMILES: *S(=O)(=O)O ; *P(=O)(O)O ; *C(=O)NO ; *C(=O)NC#N ; *c1noc(=O)[nH]1 ; *c1c(O)c(=O)c1=O]

An embodiment relates to a compound of Formula (I) as defined herein, wherein ring B is selected from 5-7 membered saturated rings, each optionally comprising one heteroatom being N, and each optionally substituted with one or more substituents independently selected from the group consisting of halogen, ═O, —OH, —CN, C₁-C₄alkyl, C₁-C₄alkyloxy, and hydroxyC₁-C₄alkyl.

An embodiment relates to a compound of Formula (I) is selected from the group consisting of compound satisfying the following general formulae:

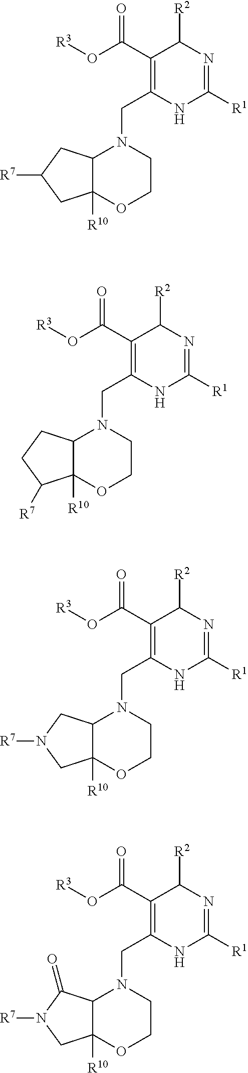

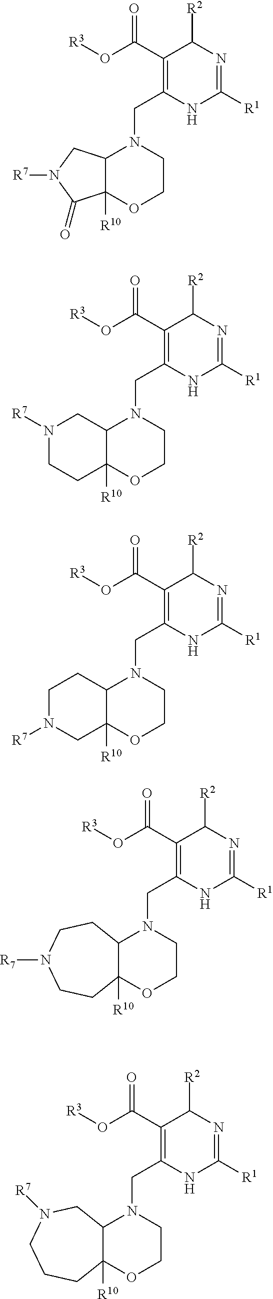

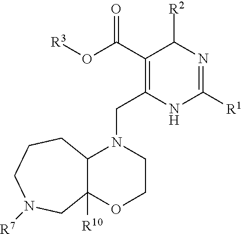

An embodiment relates to a compound of Formula (I) as defined herein, wherein wherein R¹ is selected from the group consisting of thiazolyl, pyridyl, and oxazolyl, each optionally substituted with one or more substituents selected from F, and C₁-C₆alkyl.

An embodiment relates to a compound of Formula (I) as defined herein, wherein R¹ is thiazolyl.

An embodiment relates to a compound of Formula (I) as defined herein, wherein R² is selected from the group consisting of phenyl, thiophenyl, and pyridyl, each substituted with R⁴, R⁵, and R⁶, wherein R⁴, R⁵, and R⁶ independently are selected from the group consisting of H, —OH, —CN, C₁-C₄alkyl, and halogen.

An embodiment relates to a compound of Formula (I) as defined herein, wherein R² is phenyl substituted with R⁴, R⁵, and R⁶, wherein R⁴, R⁵, and R⁶ independently are selected from the group consisting of H, —CN, —CH₃, F, Cl and Br.

An embodiment relates to a compound of Formula (I) as defined herein, wherein R³ is methyl, ethyl, propyl, or isopropyl.

An embodiment relates to a compound of Formula (I) as defined herein, wherein R⁷ is selected from the group consisting of —SO₂-Q-R⁸, —C(═O)OC₁-C₆alkyl-COOH, —C(═O)NHC₁-C₆alkyl-COOH, —C(═O)O-Q-COOH, —C(═O)NH-Q-COOH, —C(═O)C₁-C₆alkyl-R⁸, —NHC₁-C₆alkyl-R⁸, —C₁-C₆alkyl-R⁸, —(CH₂)ₚ—R⁸, and —(CH₂)ₚ-Q-R⁸.

An embodiment relates to a compound of Formula (I) as defined herein, wherein R⁸ is selected from the group consisting of —C₁-C₆alkyl-COOH, —COOH, —C(═O)NHS(═O)₂C₁-C₆alkyl, and tetrazolyl.

An embodiment relates to a compound of Formula (I) as defined herein, wherein Q is selected from the group consisting of aryl, heteroaryl, and a 3-8 membered saturated ring, each optionally substituted with R⁹, the 3-8 membered saturated ring optionally containing a heteroatom, the heteroatom being N or O; and R⁹ is selected from the group consisting of H, —C₁-C₆alkyl.

An embodiment relates to a compound of Formula (I) as defined herein, wherein p is an integer of 0, 1, or 2.

An embodiment relates to a compound is selected from the group consisting of compound satisfying the following formulae:

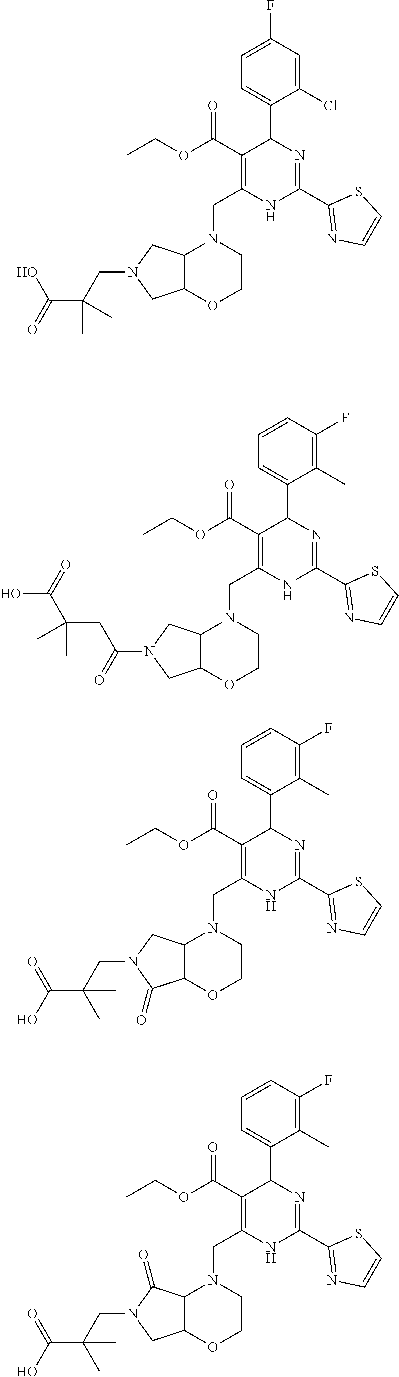 [SMILES: CCOC(=O)C1=C(CN2CCOC3CN(CC(C)(C)C(=O)O)CC32)NC(c2nccs2)=NC1c1ccc(F)cc1Cl ; CCOC(=O)C1=C(CN2CCOC3CN(C(=O)CC(C)(C)C(=O)O)CC32)NC(c2nccs2)=NC1c1cccc(F)c1C ; CCOC(=O)C1=C(CN2CCOC3C(=O)N(CC(C)(C)C(=O)O)CC32)NC(c2nccs2)=NC1c1cccc(F)c1C ; CCOC(=O)C1=C(CN2CCOC3CN(CC(C)(C)C(=O)O)C(=O)C32)NC(c2nccs2)=NC1c1cccc(F)c1C]

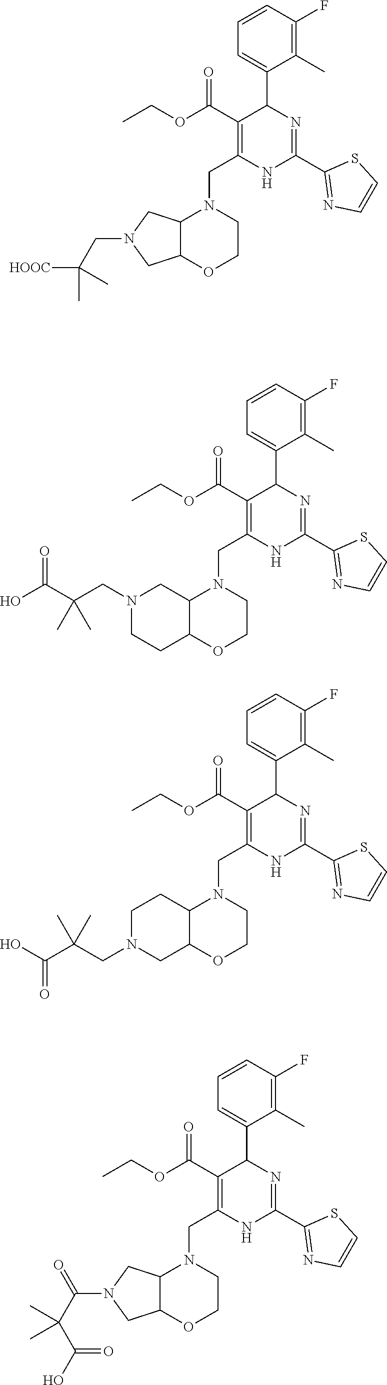 [SMILES: CCOC(=O)C1=C(CN2CCOC3CN(CC(C)(C)C(=O)O)CC32)NC(c2nccs2)=NC1c1cccc(F)c1C ; CCOC(=O)C1=C(CN2CCOC3CCN(CC(C)(C)C(=O)O)CC32)NC(c2nccs2)=NC1c1cccc(F)c1C ; CCOC(=O)C1=C(CN2CCOC3CN(CC(C)(C)C(=O)O)CCC32)NC(c2nccs2)=NC1c1cccc(F)c1C ; CCOC(=O)C1=C(CN2CCOC3CN(C(=O)C(C)(C)C(=O)O)CC32)NC(c2nccs2)=NC1c1cccc(F)c1C]

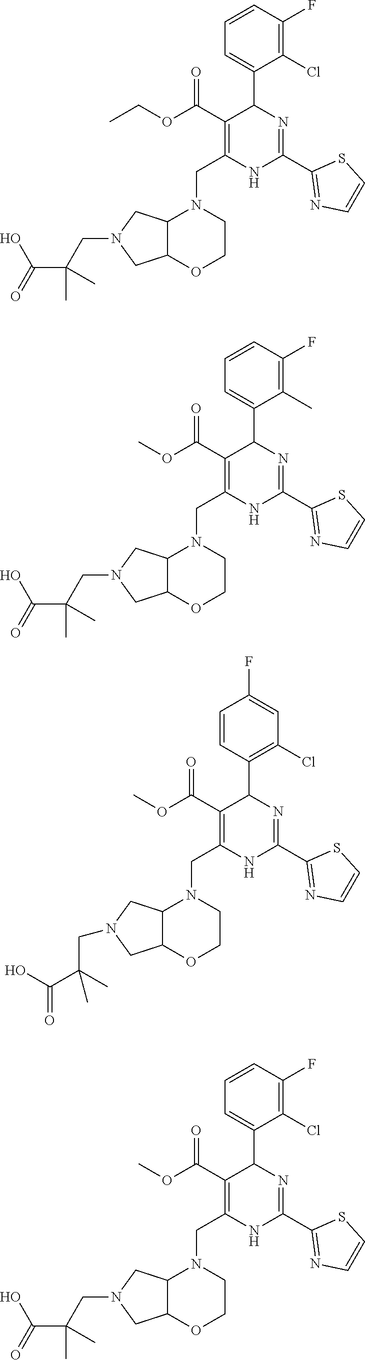 [SMILES: CCOC(=O)C1=C(CN2CCOC3CN(CC(C)(C)C(=O)O)CC32)NC(c2nccs2)=NC1c1cccc(F)c1Cl ; COC(=O)C1=C(CN2CCOC3CN(CC(C)(C)C(=O)O)CC32)NC(c2nccs2)=NC1c1cccc(F)c1C ; COC(=O)C1=C(CN2CCOC3CN(CC(C)(C)C(=O)O)CC32)NC(c2nccs2)=NC1c1ccc(F)cc1Cl ; COC(=O)C1=C(CN2CCOC3CN(CC(C)(C)C(=O)O)CC32)NC(c2nccs2)=NC1c1cccc(F)c1Cl]

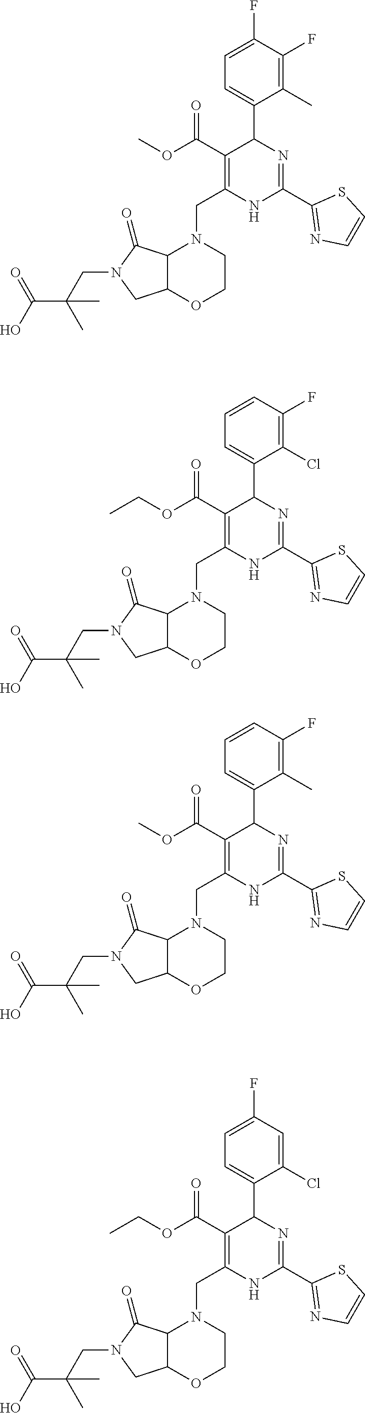 [SMILES: COC(=O)C1=C(CN2CCOC3CN(CC(C)(C)C(=O)O)C(=O)C32)NC(c2nccs2)=NC1c1ccc(F)c(F)c1C ; CCOC(=O)C1=C(CN2CCOC3CN(CC(C)(C)C(=O)O)C(=O)C32)NC(c2nccs2)=NC1c1cccc(F)c1Cl ; COC(=O)C1=C(CN2CCOC3CN(CC(C)(C)C(=O)O)C(=O)C32)NC(c2nccs2)=NC1c1cccc(F)c1C ; CCOC(=O)C1=C(CN2CCOC3CN(CC(C)(C)C(=O)O)C(=O)C32)NC(c2nccs2)=NC1c1ccc(F)cc1Cl]

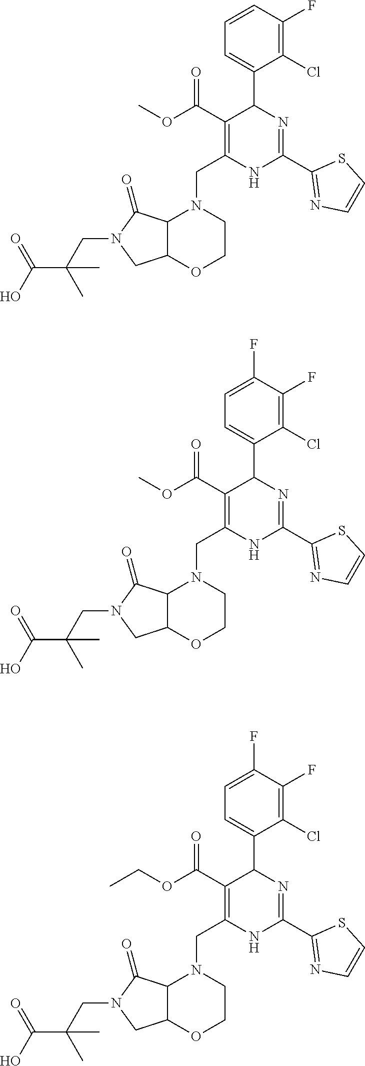 [SMILES: COC(=O)C1=C(CN2CCOC3CN(CC(C)(C)C(=O)O)C(=O)C32)NC(c2nccs2)=NC1c1cccc(F)c1Cl ; COC(=O)C1=C(CN2CCOC3CN(CC(C)(C)C(=O)O)C(=O)C32)NC(c2nccs2)=NC1c1ccc(F)c(F)c1Cl ; CCOC(=O)C1=C(CN2CCOC3CN(CC(C)(C)C(=O)O)C(=O)C32)NC(c2nccs2)=NC1c1ccc(F)c(F)c1Cl]

The disclosed compounds may possess one or more stereocenters, and each stereocenter may exist independently in either the R or S configuration. For some compounds, the stereochemical configuration at indicated centres has been assigned as “R*”, “S*”, “*R” or (*S) when the absolute stereochemistry is undetermined although the compound itself has been isolated as a single stereoisomer and is enantiomerically/diastereomerically pure. In an embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein.

Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the disclosed compound described herein. In another embodiment, compounds described herein contain one or more chiral centers. These compounds are prepared by any means, including stereoselective synthesis, enantioselective synthesis or separation of a mixture of enantiomers or diastereomers. Resolution of compounds and isomers thereof is achieved by any means including, by way of non-limiting example, chemical processes, enzymatic processes, fractional crystallization, distillation, and chromatography.

When the absolute R or S stereochemistry of a compound cannot be determined, it can be identified by the retention time after chromatography under particular chromatographic conditions as determined by chromatography column, eluent etc.

In one embodiment, the disclosed compounds may exist as tautomers. All tautomers are included within the scope of the compounds presented herein.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements).

In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In an embodiment, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and techniques known to a person skilled in the art. General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources or are prepared using procedures described herein.

### Methods

Provided herein is a method of treating an HBV infection in an individual in need thereof, comprising administering to the individual a therapeutically effective amount of a disclosed compound.

Also provided herein is a method of eradicating an HBV infection in an individual in need thereof, comprising administering to the individual a therapeutically effective amount of a disclosed compound.

Provided herein is a method of reducing viral load associated with an HBV infection in an individual in need thereof, comprising administering to the individual a therapeutically effective amount of a disclosed compound.

Further, provided herein is a method of reducing reoccurrence of an HBV infection in an individual in need thereof, comprising administering to the individual a therapeutically effective amount of a disclosed compound.

Provided herein is a method of inhibiting or reducing the formation or presence of HBV DNA-containing particles or HBV RNA-containing particles in an individual in need thereof, comprising administering to the individual a therapeutically effective amount of a disclosed compound.

In certain aspects, the methods and/or compositions described herein are effective for inhibiting or reducing the formation or presence of HBV-associated particles in vitro or in vivo (e.g., in a cell, in a tissue, in an organ (e.g., in the liver), in an organism or the like). HBV-associated particles may contain HBV DNA (i.e., linear and/or covalently closed circular DNA (cccDNA)) and/or HBV RNA (i.e., pre-genomic RNA and/or sub-genomic RNA). Accordingly, HBV-associated particles include HBV DNA-containing particles or HBV RNA-containing particles.

As used herein, “HPV-associated particles” refer to both infectious HBV virions (i.e., Dane particles) and non-infectious HBV subviral particles (i.e., HBV filaments and/or HBV spheres). HBV virions comprise an outer envelope including surface proteins, a nucleocapsid comprising core proteins, at least one polymerase protein, and an HBV genome. HBV filaments and HBV spheres comprise HBV surface proteins, but lack core proteins, polymerase and an HBV genome. HBV filaments and HBV spheres are also known collectively as surface antigen (HBsAg) particles. HBV spheres comprise middle and small HBV surface proteins. HBV filaments also include middle, small and large HBV surface proteins.

HBV subviral particles can include the nonparticulate or secretory HBeAg, which serves as a marker for active replication of HBV.

Provided herein is a method of reducing an adverse physiological impact of an HBV infection in an individual in need thereof, comprising administering to the individual a therapeutically effective amount of a disclosed compound.

Also provided herein is a method of reducing, slowing, or inhibiting an HBV infection in an individual in need thereof, comprising administering to the individual a therapeutically effective amount of a disclosed compound.

Provided herein is a method of inducing reversal of hepatic injury from an HBV infection in an individual in need thereof, comprising administering to the individual a therapeutically effective amount of a disclosed compound.

Provided herein is a method of reducing the physiological impact of long-term antiviral therapy for HBV infection in an individual in need thereof, comprising administering to the individual a therapeutically effective amount of a disclosed compound.

Provided herein is a method of prophylactically treating an HBV infection in an individual in need thereof, wherein the individual is afflicted with a latent HBV infection, comprising administering to the individual a therapeutically effective amount of a disclosed compound.

In an embodiment, the individual is refractory to other therapeutic classes of HBV drugs (e.g., HBV polymerase inhibitors, interferons, viral entry inhibitors, viral maturation inhibitors, literature-described capsid assembly modulators, antiviral compounds of distinct or unknown mechanism, and the like, or combinations thereof). In another embodiment, the disclosed method reduces viral load in an individual suffering from an HBV infection to a greater extent or at a faster rate compared to the extent that other therapeutic classes of HBV drugs reduce viral load in the individual.

In an embodiment, the administering of a disclosed compound, or a pharmaceutically acceptable salt thereof, allows for administering of the at least one additional therapeutic agent at a lower dose or frequency as compared to the administering of the at least one additional therapeutic agent alone that is required to achieve similar results in prophylactically treating an HBV infection in an individual in need thereof.

In an embodiment, the administering of a disclosed compound, or a pharmaceutically acceptable salt thereof, reduces the viral load in the individual to a greater extent or at a faster rate compared to the administering of a compound selected from the group consisting of an HBV polymerase inhibitor, interferon, viral entry inhibitor, viral maturation inhibitor, distinct capsid assembly modulator, antiviral compounds of distinct or unknown mechanism, and any combination thereof.

In an embodiment, the disclosed method reduces viral load in an individual suffering from an HBV infection, thus allowing lower doses or varying regimens of combination therapies to be used.

In an embodiment, the disclosed method causes a lower incidence of viral mutation or viral resistance compared to other classes of HBV drugs, thereby allowing for long term therapy and minimizing the need for changes in treatment regimens.

In an embodiment, the administering of a compound the invention, or a pharmaceutically acceptable salt thereof, causes a lower incidence of viral mutation or viral resistance than the administering of a compound selected from the group consisting of an HBV polymerase inhibitor, interferon, viral entry inhibitor, viral maturation inhibitor, distinct capsid assembly modulator, antiviral compounds of distinct or unknown mechanism, and combination thereof.

In an embodiment, the disclosed method increases the seroconversion rate from HBV infected to non-HBV infected or from detectable HBV viral load to non-detectable HBV viral load beyond that of current treatment regimens. As used herein, “seroconversion” refers to the period of time during which HBV antibodies develop and become detectable.

In an embodiment, the disclosed method increases or normalizes or restores normal health, elicits full recovery of normal health, restores life expectancy, or resolves the viral infection in the individual in need thereof.

In an embodiment, the disclosed method eliminates or decreases the number of HBV RNA particles that are released from HBV infected cells thus enhancing, prolonging, or increasing the therapeutic benefit of the disclosed compounds.

In an embodiment, the disclosed method eradicates HBV from an individual infected with HBV, thereby obviating the need for long term or life-long treatment, or shortening the duration of treatment, or allowing for reduction in dosing of other antiviral agents.

In another embodiment, the disclosed method further comprises monitoring or detecting the HBV viral load of the subject, and wherein the method is carried out for a period of time including until such time that the HBV virus is undetectable.

Accordingly, in an embodiment, provided herein is a method of treating an HBV infection in an individual in need thereof, comprising administering to the individual a therapeutically effective amount of a compound of Formula I, or a pharmaceutically acceptable salt thereof.

Accordingly, in an embodiment, provided herein is a method of treating an HBV infection in an individual in need thereof, comprising administering to the individual a therapeutically effective amount of a compound of Formula I, or a pharmaceutically acceptable salt thereof.

In another embodiment, provided herein is a method of treating an HBV infection in an individual in need thereof, comprising administering to the individual a therapeutically effective amount of a compound of Table 1, or a pharmaceutically acceptable salt thereof.

In an embodiment of any of the methods provided herein, the method can further comprise monitoring the HBV viral load of the subject, wherein the method is carried out for a period of time such that the HBV virus is undetectable.

### Combination Therapies

The disclosed compounds may be useful in combination with one or more additional compounds useful for treating HBV infection, or a HBV-associated or -induced disease, or a liver disease. These additional compounds may comprise other disclosed compounds and/or compounds known to treat, prevent, or reduce the symptoms or effects of HBV infection, or of an HBV-associated or -induced disease, or of a liver disease.

Particularly, in an aspect a product is provided comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the compound or pharmaceutically acceptable salt of the application or the pharmaceutical composition of the application, and wherein said second compound is another HBV inhibitor which is selected from the group consisting of HBV combination drugs, HBV DNA polymerase inhibitors, immunomodulators toll-like (TLR) receptor modulators, interferon alpha receptor ligands, hyaluronidase inhibitors, hepatitis b surface antigen (HbsAg) inhibitors, cytotoxic T-lymphocyte-associated protein 4 (ipi4) inhibitors, cyclohilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, farnsoid X receptor agonists, HBV antibodies, CCR2 chemokine antagonists, thymosin agonists, cytokines, nuceloprotein modulators, retinoic acid-inducible gene 1 stimulators, NOD2 stimulators, phosphatidylinositol 3-kinase (P13K) inhibitors, indoleamine 2,3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and (other) anti-HBV drugs.

The one or more additional compounds may e.g., be selected from interferon (for example, interferon-alpha-2a is pegylated interferon-alpha-2a (PEGASYS)), nucleoside or nucleotide or non-nucleos(t)ide polymerase inhibitors, immunomodulatory agents (e.g., IL-12, IL-18, IFN-alpha, -beta, and -gamma and TNF-alpha among others), TLR agonists, siRNAs and antisense oligonucleotides.

In another embodiment, the disclosed compound and the at least one additional therapeutic agent are co-formulated. In yet another embodiment, the disclosed compound and the at least one additional therapeutic agent are co-administered.

For any combination therapy described herein, synergistic effect may be calculated, for example, using suitable methods such as the Sigmoid-Eurax equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In an embodiment of any of the methods of administering combination therapies provided herein, the method can further comprise monitoring or detecting the HBV viral load of the subject, wherein the method is carried out for a period of time including until such time that the HBV virus is undetectable.

### Administration/Dosage/Formulations

In another aspect, provided herein is a pharmaceutical composition comprising at least one disclosed compound, or a pharmaceutically acceptable salt thereof, together with a pharmaceutically acceptable carrier.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

In particular embodiments, the compound is formulated in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms of the invention are dictated by and directly dependent on (a) the unique characteristics of the disclosed compound and the particular therapeutic effect to be achieved, and (b) the limitations inherent in the art of compounding/formulating such a disclosed compound for the treatment of HBV infection in a patient.

In an embodiment, the compositions of the invention are formulated using one or more pharmaceutically acceptable excipients or carriers. In an embodiment, the pharmaceutical compositions of the invention comprise a therapeutically effective amount of a disclosed compound and a pharmaceutically acceptable carrier.

In some embodiments, the dose of a disclosed compound is from about 1 mg to about 2,500 mg. In some embodiments, a dose of a disclosed compound used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., another drug for HBV treatment) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In an embodiment, the present invention is directed to a packaged pharmaceutical composition comprising a container holding a therapeutically effective amount of a disclosed compound, alone or in combination with a second pharmaceutical agent; and instructions for using the compound to treat, prevent, or reduce one or more symptoms of HBV infection in a patient.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be un-coated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The term “comprising”, which is synonymous with “including” or “containing”, is open-ended, and does not exclude additional, unrecited element(s), ingredient(s) or method step(s), whereas the term “consisting of” is a closed term, which excludes any additional element, step, or ingredient which is not explicitly recited.

The term “essentially consisting of” is a partially open term, which does not exclude additional, unrecited element(s), step(s), or ingredient(s), as long as these additional element(s), step(s) or ingredient(s) do not materially affect the basic and novel properties of the invention.

The term “comprising” (or “comprise(s)”) hence includes the term “consisting of” (“consist(s) of”), as well as the term “essentially consisting of” (“essentially consist(s) of”). Accordingly, the term “comprising” (or “comprise(s)”) is, in the present application, meant as more particularly encompassing the term “consisting of” (“consist(s) of”), and the term “essentially consisting of” (“essentially consist(s) of”).

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated. Each of the relevant disclosures of all references cited herein is specifically incorporated by reference. The following examples are offered by way of illustration, and not by way of limitation.

### EXAMPLES

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

### PREPARATIVE EXAMPLES

Unless otherwise indicated, LCMS and NMR was conducted by using one of the following general methods.

General Methods of LCMS and NMR

General Procedure A

The LCMS measurement was performed using an Agilent system comprising a binary pump with degasser, an autosampler, a column oven (set at 40° C., unless otherwise indicated) and a column as specified in the respective methods below. Flow from the column was split to a MS and UV spectrometer. The MS detector was configured with an electrospray ionization source. Mass spectra were acquired by scanning from 100 to 1000 in 1.06 sec/cycle. The capillary voltage was 3 kV for positive ionization mode and 2.5 kV for negative ionization mode and the source temperature was maintained at 100° C. Nitrogen was used as the nebulizer gas. Data acquisition was performed with an Agilent ChemStation data system.

Method 1

In addition to the general procedure A: reversed phase LCMS for quality control was performed by Agilent 1200 with a diode-array detector (DAD) and carried out on a Sunfire C18 column (5 μm, 4.6×50 mm) with a flow rate of 1.5 ml/min. Two mobile phases (mobile phase A1: 0.02% ammoniumacetate in water; mobile phase A2: 0.1% TFA in water; mobile phase B1: acetonitrile) were employed to run a gradient condition from 95% A1 or A2 and 5% B to 5% A1 or A2 and 95% B in 4.0 minutes. An injection volume of 1˜10 μl was used.

Method 2

In addition to the general procedure A: reversed phase LCMS for monitoring the reactions was performed by Agilent 1260 with a variable wavelength detector (VWD) and carried out on a Dikma Diamonsil plus C18 column (5 μm, 4.6×30 mm) with a flow rate of 2.0 ml/min. Two mobile phases (mobile phase A1: H₂O+0.02% ammoniumacetate+5% ACN; mobile phase A2: H₂O+0.1% TFA+5% ACN; mobile phase B: acetonitrile) were employed to run a gradient condition from 95% A1 or A2 and 5% B to 5% A1 or A2 and 95% B in 1.4 minutes. An injection volume of 1˜5 μl was used.

Method 3

In addition to the general procedure A: reversed phase LCMS for monitoring the reactions was performed by Agilent 6120 (stationary phase Sunfire C18 2.5 μm, 3.0×30 mm. Mobile phase: 0.01% FA solution in water, and ACN, Gradient from 5% ACN to 95% in 2.5 min and stay in 95% for 1 min.

General Procedure B

The LCMS measurement was performed using a UPLC (Ultra Performance Liquid Chromatography) Acquity (Waters) system comprising a quaternary pump with degasser, an autosampler, a photo-diode array detector (PDA) and a column as specified in the respective methods below, the column is hold at a temperature of 40° C. Flow from the column was brought to MS detector. The MS detector was configured with an electrospray ionization source. Mass spectra were acquired by scanning from 100 to 1000 in 0.25 sec/cycle. The capillary needle voltage was 3 kV and the source temperature was maintained at 120° C. Cone voltage was 30 V for positive ionization mode and 30 V for negative ionization mode. Nitrogen was used as the nebulizer gas. Data acquisition was performed with a Waters-Micromass MassLynx-Openlynx data system.

Reversed phase UPLC was carried out on a Waters Acquity BEH (bridged ethylsiloxane/silica hybrid) C18 column (1.7 μm, 2.1×50 mm) with a flow rate of 0.5 ml/min. Two mobile phases (mobile phase A: 95% (H₂O+0.02% ammoniumacetate+5% ACN); mobile phase B: acetonitrile; mobile phase C: 95% (H₂O+0.1% TFA+5% ACN) were employed to run a gradient condition from 95% A or C and 5% B to 5% A or C and 95% B in 1 minute. An injection volume of 0.5 μl was used.

General Procedure C

The reversed phase preparation was performed using a system comprising two unit pumps without degasser, a UV/Vis detector and a column as specified in the respective methods below. Flow from the column was split to a UV spectrometer.

Method 1

In addition to the general procedure C: Prep-reversed phase LC was carried out on a Gilson with an autosampler, an Xbridge prep C18 OBD column (5 μm, 19×150 mm) with a flow rate of 15-20 ml/min. Two mobile phases (mobile phase A1: H₂O (0.1% Ammonium bicarbonate); mobile phase A2: H₂O (Ammonium hydroxide);

mobile phase A3: H₂O (0.1% TFA); mobile phase B: acetonitrile) were employed to run a gradient condition from 95% A1 or A2 or A3 and 5% B to 20% A1 or A2 or A3 and 80% B. Data acquisition was performed with a Trilution LC data system.

Method 2

In addition to the general procedure C: reversed phase preparation was carried out on a automatic medium pressure flash separation-Compact Purifier from Lisure Science Ltd. with reversed phase SW-5231 C18 column (40-60 μm, 120 Å, 18 g, 40 g, 130 g) with a flow rate of 30-100 ml/min. Two mobile phases (mobile phase A1: H₂O (0.1% Ammonium bicarbonate); mobile phase A2: H₂O (Ammonium hydroxide); mobile phase A3: H₂O (0.1% Hydrochloric acid); mobile phase A4: H₂O; mobile phase B: acetonitrile) were employed to run a gradient condition from 95% A1 or A2 or A3 or A4 and 5% B to 5% A1 or A2 or A3 or A4 and 95% B. Data acquisition was performed with a Compact data system.

Method 3

In addition to the general procedure C: Prep-reversed phase LC was carried out on a Waters with an autosampler, a Xbridge prep C18 OBD column (Sum, 19*150 mm) with a flow rate of 20 ml/min. Two mobile phases (mobile phase A: H₂O (0.1% Ammonium bicarbonate); mobile phase B: acetonitrile) were employed to run a gradient condition from 95% A and 5% B to 50% A and 50% B. Data acquisition was performed with a Waters MassLynx data system.

General Procedure D

The chiral measurement was performed using a system comprising an autosampler, a column oven (set at ambient, unless otherwise indicated), a diode-array detector (DAD) and a column as specified in the respective methods below. Flow from the column was split to a UV spectrometer. LC spectra were acquired by scanning from 190 nm to 400 nm with deuterium lamp and from 401 nm to 800 nm with tungsten lamp using a slit width of 1.2 nm. The chiral chiralpak or chiralcel columns from Daicel Chiral technologies (China) Ltd. are divided into two types according to the different stuffings: Type 1: IA, IB, IC, ID, IE, IF, IG, IH; Type 2: AD-H, AS-H, OD-H, OJ-H.

Method 1:

In addition to the general procedure D: Chiral HPLC was carried out on an Agilent 1200 or Shimadzu LC-20A with a quaternary pump with degasser, a chiral column (Sum, 4.6*250 mm) with a flow rate of 1.0 ml/min for chiral analysis or a chiral column (Sum, 20*250 mm) with a flow rate of 10-20 ml/min for chiral preparation. The mobile phases are the different ratios among MeOH, EtOH, Hex, IPA etc. Data acquisition was performed with an Agilent ChemStation or Shimadzu LabSolutions data system.

Method 2:

In addition to the general procedure D: chiral analysis was carried out on a Waters-TharSFC with a column oven (40° C.) with a flow rate of 2-3 ml/min and data acquisition was performed with TharSFC Chrom Scope data system. Chiral-preparation was carried out on a Waters-SFC-80 with a flow rate of 45-60 ml/min and data acquisition was performed with Waters-TharSFC SuperChrom data system. The mobile phase is CO2 and MeOH, EtOH can be used as co-solvents.

General Procedure E

The below NMR experiments were carried out using a NMR spectrometers at ambient temperature, using internal deuterium lock and equipped with BBO 400 MHz S1 5 mm with Z-gradient; PLUS (²H, ¹H, BBF) probe head for the 400 MHz and DUL 300 MHz S1 5 mm Z-gradient (²H, ¹H, ¹³C) probe head for the 300 MHz. Chemical shifts (δ) are reported in parts per million (ppm).

Method 1:

In addition to the general procedure E: A Bruker Avance III 400 MHz spectrometer was used to measure the NMR experiment.

Method 2:

In addition to the general procedure E: A Bruker Avance Neo 400 MHz spectrometer was used to measure the NMR experiment.

Method 3:

In addition to the general procedure E: A ZKNJ BIXI-1 300 MHz spectrometer was used to measure the NMR experiment.

Method 4:

In addition to the general procedure E: A Bruker Ascend 400 MHz spectrometer was used to measure the NMR experiment.

Exemplary compounds useful in methods of the invention will now be described by reference to the illustrative synthetic schemes for their general preparation below and the specific examples to follow.

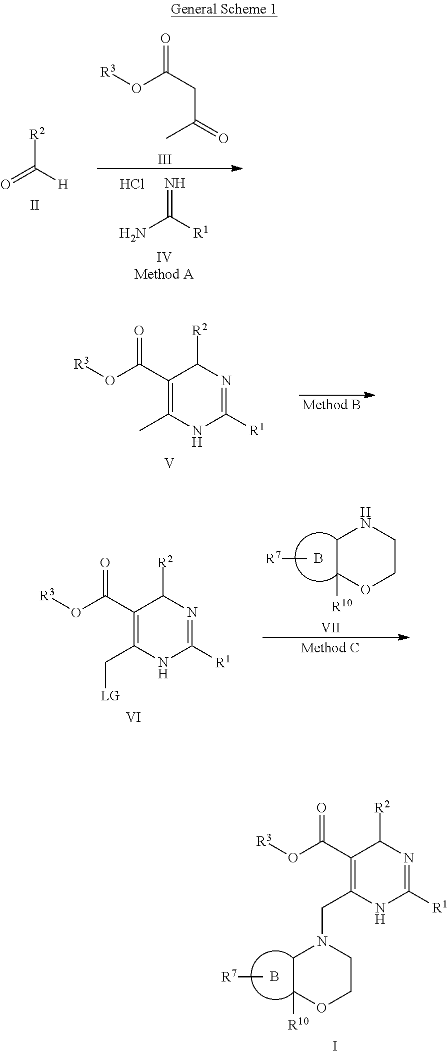

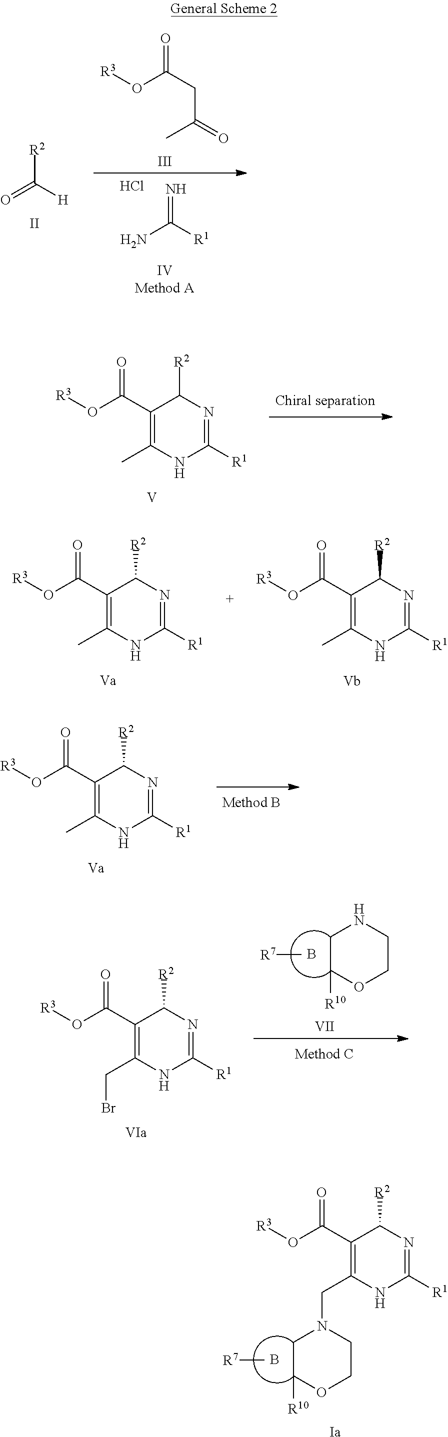

The preparation of compound I is shown in the above Scheme 1 and Scheme 2. Compound V can be prepared by the condensation of aldehyde II, acetoacetate III and amidine IV in the presence of a base such as NaOAc (Method A). Compound VI, wherein LG represents a leaving group, such as bromo, can be prepared from compound V using brominating reagent such as N-Bromosuccinimide (Method B). Coupling of compounds VI and compound VII in the presence of a base such as triethanolamine affords compound I (Method C). Alternatively, compound V can be subjected to chiral separation to give its single stereoisomer compound Va and compound Vb, compound VIa was prepared from compound Va using brominating reagent such as N-Bromosuccinimide (Method B). Coupling of compounds VIa and compound VII in the presence of a base such as triethanolamine affords compound Ia (Method C).

### Method A:

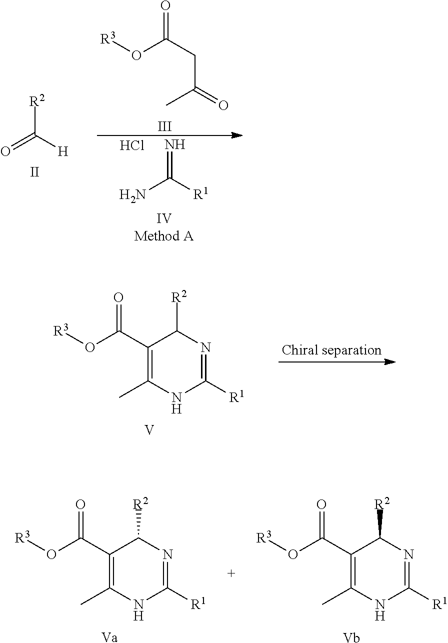

To a solution of the ketoester of general formula III (1 equivalent) in solvent such as ethanol or methanol was added the aldehyde of general formula II (1 equivalent), the carboxamidine hydrochloride of general formula IV (1 equivalent) and a base such as sodium acetate (1-1.2 equivalents). The mixture was brought up to 70-100° C. and stirred under nitrogen atmosphere from six hours to overnight. After cooled down to room temperature, it was concentrated to dryness. The residue was extracted from dichloromethane or ethyl acetate, washed with water, brine, dried over anhydrous Na₂SO₄ and filtered. The filtrate was concentrated under reduced pressure to give a residue, which was purified by silica gel column chromatography to afford the dihydropyrimidine product of general formula V. When applicable, the stereoisomers of the dihydropyrimidine product of general formula V was isolated and purified using chiral chromatography to give the dihydropyrimidine products of general formula Va and general formula Vb.

### Method B:

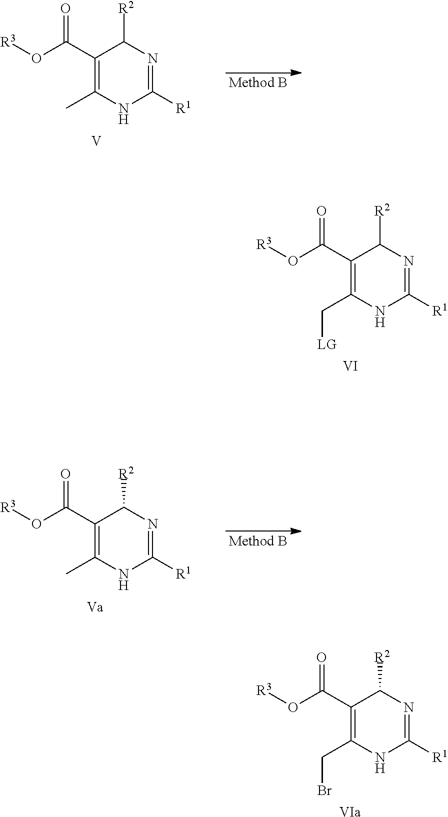

To a solution of the dihydropyrimidine of general formula V or Va (1 equivalent) in solvent such as carbon tetrachloride was added brominating reagent such as N-bromosuccinimide (0.9 to 1.1 equivalent) at room temperature and nitrogen atmosphere. The mixture was brought up to 60° C. and stirred under nitrogen atmosphere for 1 hour. After cooled down to room temperature, it was concentrated to dryness. The residue was extracted from dichloromethane or ethyl acetate, washed with water, brine, dried over anhydrous Na₂SO₄ and filtered. The filtrate was concentrated under reduced pressure to give a residue, which was purified by silica gel column chromatography to afford the dihydropyrimidine product of general formula VI or VIa, respectively.

### Method C:

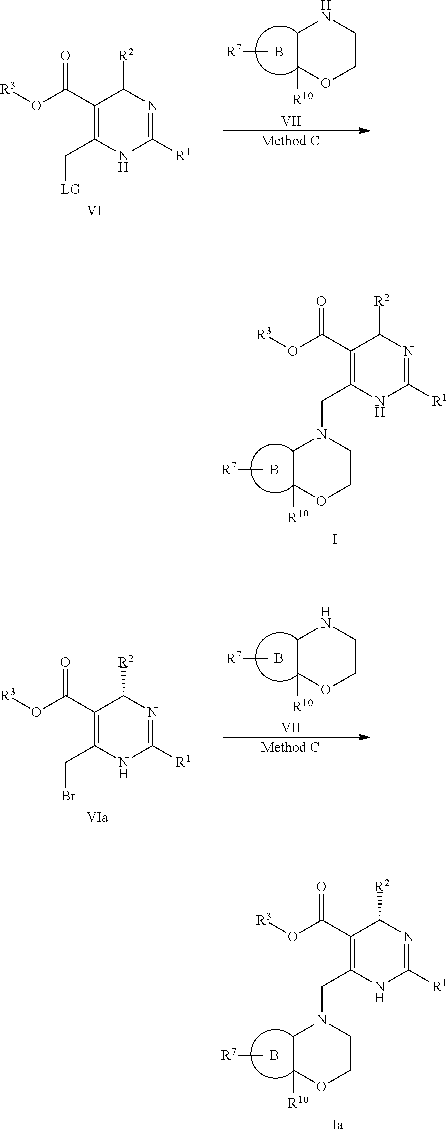

To a solution of general formula VII (1 equivalent) in solvent such as dichloromethane was added a base such as triethanolamine (10 equivalent) at room temperature. The mixture was stirred at 35° C. for 0.5 hour and then a solution of the dihydropyrimidine of general formula VI or VIa (1 equivalent) in solvent such as dichloromethane was added at nitrogen atmosphere. The mixture was brought up to 40° C. and stirred under nitrogen atmosphere for 2 hours. After cooled down to 0° C., the reaction mixture was poured into a mixture of ice-water and aqueous hydrochloride solution (1.0 M). The resulting mixture was extracted from dichloromethane or ethyl acetate, washed with water, brine, dried over anhydrous Na₂SO₄ and filtered. The filtrate was concentrated under reduced pressure to give a residue, which was purified by C18 column to afford the dihydropyrimidine product of general formula I or Ia, respectively.

### Chemistry

Several methods for preparing the compounds of this invention are illustrated hereinbelow. Unless otherwise noted, all starting materials were obtained from commercial suppliers and used without further purification.

Hereinafter, ACN means acetonitrile, AcOH means acetic acid, Boc means tert-butyloxycarbonyl, Bn means benzyl, calcd. means calculated, Cbz means benzyloxycarbonyl, col. means column, conc. means concentrated, m-CPBA means 3-chloroperbenzoic acid, DAST means (diethylamino)sulfur trifluoride, DCM means dichloromethane, DEA means diethanolamine, DIEA means N,N-diisopropylethyl amine, DMAP means 4-(dimethylamino)pyridine, DMF means dimethylformamide, DMP means Dess-Martin periodinane, EA means ethyl acetate, ee means enantiomeric excess, ESI means electrospray ionization, HATU means 2-(7-azabenzotriazol-1-yl)-N,N,N′,N′-tetramethyluronium hexafluorophosphate, Hex means hexane, HNMR means ¹H NMR, HPLC means high performance liquid chromatography, IPA means isopropyl alcohol, LC-MS or LCMS means liquid chromatography-mass spectrometry, LDA means lithium diisopropylamide, Ms means methanesulfonyl, PE means petroleum ether, PMB means 4-methoxybenzyl, prep. means preparative, Prep-HPLC means preparative HPLC, R_{T} or Rt mean retention time, ₍ₛ₎ or (s) mean solid, sat. means saturated, TBAF means tetrabutylammonium fluoride, TBS means tert-butyldimethylsilyl, TEOA means triethanolamine, TEA means triethylamine, THF means tetrahydrofuran, T or Temp mean temperature, TsCl means 4-toluenesulfonyl chloride, t-BuOK means potassium tert-butoxide, W means wavelength.

### EXAMPLES

### Example 1

### Compound VIa-1: (R*)-ethyl 6-(bromomethyl)-4-(2-chloro-4-fluorophenyl)-2-(thiazol-2-yl)-1,4-dihydropyrimidine-5-carboxylate (a Single Stereoisomer)

### Intermediate V-1: Ethyl 4-(2-chloro-4-fluorophenyl)-6-methyl-2-(thiazol-2-yl)-1,4-dihydropyrimidine-5-carboxylate (a Mixture of 2 stereoisomers) (Exemplified with Method A)

To a solution of 2-chloro-4-fluorobenzaldehyde (10.0 g, 63.1 mmol) in ethanol (200 mL) was added ethyl 3-oxobutanoate (8.20 g, 63.1 mmol), thiazole-2-carboximidamide hydrochloride (10.3 g, 63.1 mmol) and sodium acetate (5.20 g, 63.1 mmol) at room temperature. The mixture was stirred at 80° C. overnight. It was cooled to room temperature, extracted with ethyl acetate, washed with brine, dried over Na₂SO₄ and purified by silica gel column chromatography (petroleum ether:ethyl acetate=10:1 to 1:1) to give the title compound (14.0 g, 90% purity, 58% yield) as yellow solids. LC-MS (ESI): R_{T}=1.75 min, mass calcd. for C₁₇H₁₅ClFN₃O₂S 379.1, m/z found 380.0 [M+H]⁺. ¹H NMR (400 MHz, CDCl₃) δ 7.83 (d, J=3.2 Hz, 0.3H), 7.81-7.80 (m, 1.4H), 7.50 (d, J=3.6 Hz, 0.3H), 7.46 (br s, 0.3H), 7.43 (d, J=3.2 Hz, 0.7H), 7.36-7.32 (m, 1H), 7.14-7.11 (m, 1H), 6.94-6.89 (m, 1H), 6.20 (s, 0.7H), 6.08 (s, 0.3H), 4.10-4.01 (m, 2H), 2.57 (s, 0.7H), 2.51 (s, 2.3H), 1.15-1.11 (t, J=7.2 Hz, 3H).

A racemic mixture of ethyl 4-(2-chloro-4-fluorophenyl)-6-methyl-2-(thiazol-2-yl)-1,4-dihydropyrimidine-5-carboxylate V-1(1.00 g, 90% purity, 2.37 mmol) was separated by chiral Prep. HPLC (separation condition: Column: Chiralpak IE 5 μm 20*250 mm; Mobile Phase: Hex:EtOH=90:10 at 10 mL/min; Temp: 30° C.; Wavelength: 254 nm) to give the title compounds Va-1 (400 mg, 98.1% purity, 44% yield, 100% ee) and Vb-1 (405 mg, 98.6% purity, 40% yield, 99.7% ee) as yellow solids.

Intermediate Va-1: LC-MS (ESI): R_{T}=4.295 min, mass calcd. for C₁₇H₁₅ClFN₃O₂S 379.1, m/z found 380.1 [M+H]⁺. Chiral analysis (Column: Chiralpak IE 5 μm 4.6*250 mm; Mobile Phase: Hex:EtOH=90:10 at 1.0 mL/min; Temp: 30° C.; Wavelength: 254 nm; R_{T}=7.663 min). ¹H NMR (400 MHz, CDCl₃) δ 7.83 (d, J=3.2 Hz, 0.3H), 7.80 (d, J=2.8 Hz, 1H), 7.50 (d, J=3.2 Hz, 0.3H), 7.43 (d, J=3.2 Hz, 1H), 7.36-7.32 (m, 1H), 7.14-7.11 (m, 1H), 6.94-6.89 (m, 1H), 6.20 (s, 0.7H), 6.08 (s, 0.3H), 4.08-4.01 (m, 2H), 2.57 (s, 0.8H), 2.51 (s, 2.2H), 1.13 (t, J=7.2 Hz, 3H).

Intermediate Vb-1: LC-MS (ESI): R_{T}=3.578 min, mass calcd. for C₁₇H₁₅ClFN₃O₂S 379.1, m/z found 380.1 [M+H]⁺. Chiral analysis (Column: Chiralpak IE 5 μm 4.6*250 mm; Mobile Phase: Hex:EtOH=90:10 at 1.0 mL/min; Temp: 30° C.; Wavelength: 254 nm; R_{T}=9.471 min). ¹H NMR (400 MHz, CDCl₃) δ 7.83 (d, J=3.2 Hz, 0.3H), 7.80 (d, J=2.8 Hz, 1H), 7.50 (d, J=3.2 Hz, 0.3H), 7.43 (d, J=3.2 Hz, 1H), 7.36-7.32 (m, 1H), 7.14-7.11 (m, 1H), 6.94-6.89 (m, 1H), 6.20 (s, 0.7H), 6.08 (s, 0.3H), 4.08-4.00 (m, 2H), 2.57 (s, 0.8H), 2.51 (s, 2.2H), 1.13 (t, J=7.2 Hz, 3H).

### Compound VIa-1: (R*)-ethyl 6-(bromomethyl)-4-(2-chloro-4-fluorophenyl)-2-(thiazol-2-yl)-1,4-dihydropyrimidine-5-carboxylate (a Single Stereoisomer) (Exemplified with Method B)

To a solution of (R*)-ethyl 4-(2-chloro-4-fluorophenyl)-6-methyl-2-(thiazol-2-yl)-1,4-dihydropyrimidine-5-carboxylate Va-1 (200 mg, 98.1% purity, 0.518 mmol) in carbon tetrachloride (15 mL) was added N-bromosuccinimide (100 mg, 0.579 mmol). After stirred at 65° C. for 0.5 hour, the reaction was cooled down to room temperature, diluted with dichloromethane (20 mL), washed with water (10 mL) twice, brine (10 mL), dried over Na₂SO₄(s), filtered and evaporated under reduce pressure to remove solvent. The residue was purified by Pre-TLC (petroleum ether:ethyl acetate=5:1) to give the desired compound (137 mg, 98% purity, 57% yield) as yellow solids. LC-MS (ESI): R_{T}=1.80 min, mass calcd. for C₁₇H₁₄BrClFN₃O₂S 457.0, m/z found 458.0 [M+H]⁺.

### Preparation of Dihydropyrimidines of General Formula Va/VIa Incorporated with Aryl Aldehydes (II), Ketoester (III) and Carboxamidines (IV) Via Sequential Reactions are Shown Below in Table 1

TABLE 1

| Aldehyde of | Ketoester of | Amidine of | | Bromo- |
|---|---|---|---|---|
| general | general | general | Intermediate of | intermediate of |
| formula II | formula III | formula IV | general formula Va | general formula VIa |
| 2-chloro-4- fluorobenzaldehyde | ethyl 3- oxobutanoate | thiazole-2- carboximidamide hydrochloride | 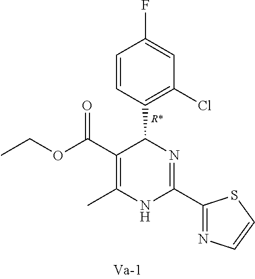 [SMILES: CCOC(=O)C1=C(C)NC(c2nccs2)=N[C@H]1c1ccc(F)cc1Cl] | 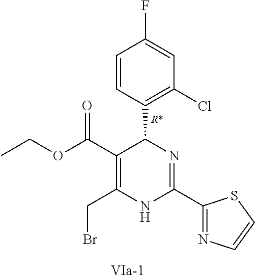 [SMILES: CCOC(=O)C1=C(CBr)NC(c2nccs2)=N[C@H]1c1ccc(F)cc1Cl] |
| 2-methyl-3- fluorobenzaldehyde | Ethyl 3- oxobutanoate | thiazole-2- carboximidamide hydrochloride | 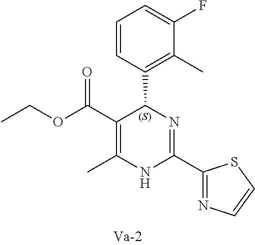 | 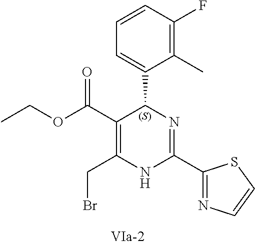 |
| 2-chloro-3- fluorobenzaldehyde | Ethyl 3- oxobutanoate | thiazole-2- carboximidamide hydrochloride | 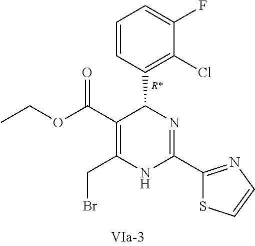 [SMILES: CCOC(=O)C1=C(CBr)NC(c2nccs2)=N[C@H]1c1cccc(F)c1Cl] | 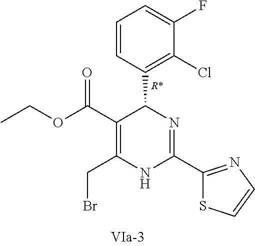 [SMILES: CCOC(=O)C1=C(CBr)NC(c2nccs2)=N[C@H]1c1cccc(F)c1Cl] |
| 2-methyl-3- fluorobenzaldehyde | Methyl 3- oxobutanoate | thiazole-2- carboximidamide hydrochloride | 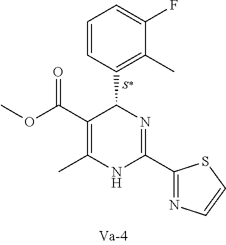 [SMILES: COC(=O)C1=C(C)NC(c2nccs2)=N[C@H]1c1cccc(F)c1C] | 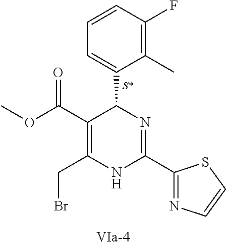 [SMILES: COC(=O)C1=C(CBr)NC(c2nccs2)=N[C@H]1c1cccc(F)c1C] |
| 2-chloro-4- fluorobenzaldehyde | Methyl 3- oxobutanoate | thiazole-2- carboximidamide hydrochloride | 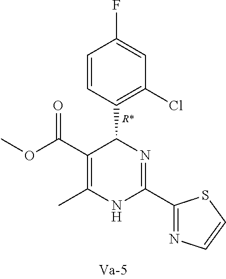 [SMILES: COC(=O)C1=C(C)NC(c2nccs2)=N[C@H]1c1ccc(F)cc1Cl] | 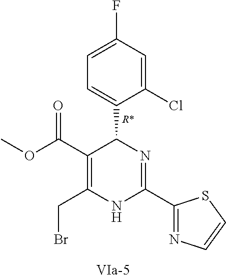 [SMILES: COC(=O)C1=C(CBr)NC(c2nccs2)=N[C@H]1c1ccc(F)cc1Cl] |
| 2-chloro-3- fluorobenzaldehyde | Methyl 3- oxobutanoate | thiazole-2- carboximidamide hydrochloride | 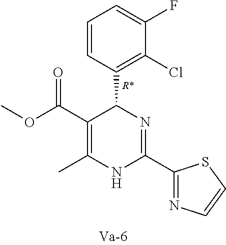 [SMILES: COC(=O)C1=C(C)NC(c2nccs2)=N[C@H]1c1cccc(F)c1Cl] | 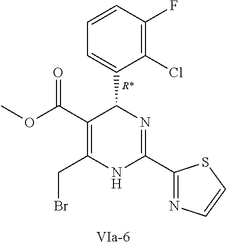 [SMILES: COC(=O)C1=C(CBr)NC(c2nccs2)=N[C@H]1c1cccc(F)c1Cl] |
| 2-methyl-3,4- difluorobenzaldehyde | Methyl 3- oxobutanoate | thiazole-2- carboximidamide hydrochloride | 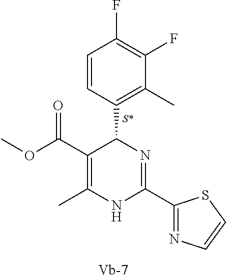 [SMILES: COC(=O)C1=C(C)NC(c2nccs2)=N[C@H]1c1ccc(F)c(F)c1C] | 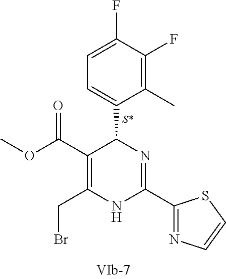 [SMILES: COC(=O)C1=C(CBr)NC(c2nccs2)=N[C@H]1c1ccc(F)c(F)c1C] |
| 2-chloro-3,4- difluorobenzaldehyde | Methyl 3- oxobutanoate | thiazole-2- carboximidamide hydrochloride | 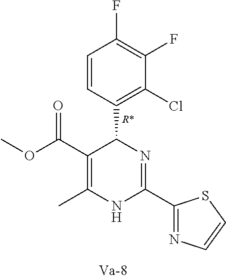 [SMILES: COC(=O)C1=C(C)NC(c2nccs2)=N[C@H]1c1ccc(F)c(F)c1Cl] | 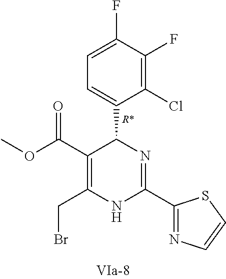 [SMILES: COC(=O)C1=C(CBr)NC(c2nccs2)=N[C@H]1c1ccc(F)c(F)c1Cl] |
| 2-chloro-3,4- difluorobenzaldehyde | ethyl 3- oxobutanoate | thiazole-2- carboximidamide hydrochloride | 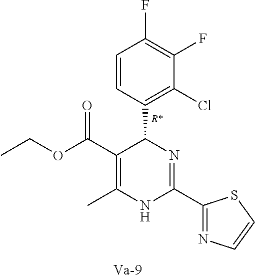 [SMILES: CCOC(=O)C1=C(C)NC(c2nccs2)=N[C@H]1c1ccc(F)c(F)c1Cl] | 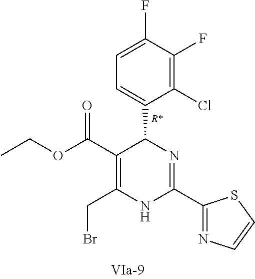 [SMILES: CCOC(=O)C1=C(CBr)NC(c2nccs2)=N[C@H]1c1ccc(F)c(F)c1Cl] |
| 2-chloro-4- fluorobenzaldehyde | Methyl 3- oxobutanoate | 3,5- difluoropicolinimid- amide hydrochloride | 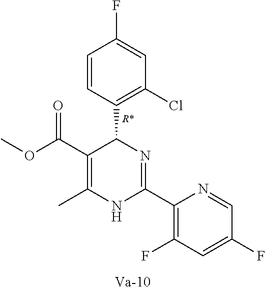 [SMILES: COC(=O)C1=C(C)NC(c2ncc(F)cc2F)=N[C@H]1c1ccc(F)cc1Cl] | 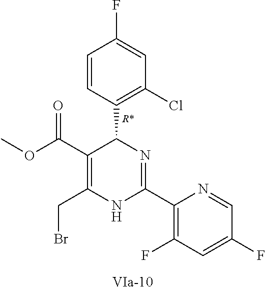 [SMILES: COC(=O)C1=C(CBr)NC(c2ncc(F)cc2F)=N[C@H]1c1ccc(F)cc1Cl] |
| 2-chloro-4- fluorobenzaldehyde | Methyl 3- oxobutanoate | 3,5- difluoropicolinimid- amide hydrochloride | 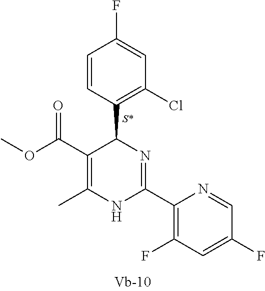 [SMILES: COC(=O)C1=C(C)NC(c2ncc(F)cc2F)=N[C@@H]1c1ccc(F)cc1Cl] | 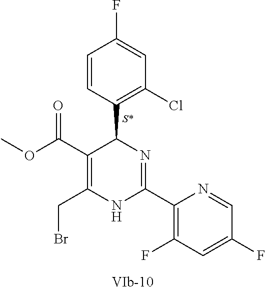 [SMILES: COC(=O)C1=C(CBr)NC(c2ncc(F)cc2F)=N[C@@H]1c1ccc(F)cc1Cl] |

### Intermediate V-2: Ethyl 4-(3-fluoro-2-methylphenyl)-6-methyl-2-(thiazol-2-yl)-1,4-dihydropyrimidine-5-carboxylate (a Mixture of 2 Stereoisomers)

By utilizing the analogous procedure of Method A, the title compound was synthesized as yellow solids.

¹H NMR (400 MHz, DMSO-d₆) δ 9.86 (s, 0.8H), 9.52 (d, J=2.8 Hz, 0.2H), 8.00-7.98 (m, 0.4H), 7.96 (d, J=3.2 Hz, 0.8H), 7.88 (d, J=2.8 Hz, 0.8H), 7.20-7.15 (m, 1.2H), 7.06-6.99 (m, 1.8H), 5.83 (s, 0.8H), 5.73 (d, J=3.2 Hz, 0.2H), 3.99-3.93 (m, 2H), 2.48 (s, 2.4H), 2.45 (s, 1.2H), 2.44 (s, 1.2H), 2.41 (s, 0.3H), 2.40 (s, 0.3H), 2.37 (s. 0.6H), 1.08-1.02 (m, 3H).

Intermediate V-2 was separated by chiral Prep-HPLC (separation condition: Column: Chiralpak OJ-H 5 μm 20*250 mm; Mobile Phase: Hex:EtOH:DEA=90:10:0.3 at 15 mL/min; Temp: 30° C.; Wavelength: 214 nm) to afford Va-2 and Vb-2 as yellow solids.

Intermediate Va-2: Chiral analysis (Column: Chiralpak OJ-H 5 μm 4.6*250 mm; Mobile Phase: Hex:EtOH:DEA=85:15:0.2 at 1.0 mL/min; Temp: 30° C.; Wavelength: 230 nm, R_{T}=7.251 min). Va-2 was certificated to absolute S stereochemistry by the following chemical resolution which is consistent with reported data (I Med. Chem., 2017, 60 (8), pp 3352-3371). Optical rotation: [a]_{D}²⁰−24° (c 0.10, MeOH).

Intermediate Vb-2: Chiral analysis (Column: Chiralpak OJ-H 5 μm 4.6*250 mm; Mobile Phase: Hex:EtOH:DEA=85:15:0.2 at 1.0 mL/min; Temp: 30° C.; Wavelength: 230 nm, R_{T}=9.072 min). Optical rotation: [a]_{D}²⁰+35° (c 0.10, MeOH).

### Intermediate VIa-2: (S)-Ethyl 6-(bromomethyl)-4-(3-fluoro-2-methylphenyl)-2-(thiazol-2-yl)-1,4-dihydropyrimidine-5-carboxylate (a Single Stereoisomer)

By utilizing the analogous procedure of Method B, the title compound was synthesized from Va-2 as yellow solids.

LC-MS (ESI): R_{T}=1.84 min, mass calcd. for C₁₈H₁₇BrFN₃O₂S 437.0, m/z found 440.0 [M+H]⁺. ¹H NMR (400 MHz, CDCl₃) δ 8.22 (s, 0.5H), 7.82 (d, J=3.2 Hz, 1H), 7.53 (s, 0.4H), 7.44 (s, 0.6H), 7.25-7.08 (m, 2.5H), 6.96-6.92 (s, 1H), 5.99 (s, 0.6H), 5.93 (s, 0.4H), 4.92-4.77 (m, 1.6H), 4.67-4.65 (m, 0.4H), 4.13-4.07 (m, 2H), 2.53 (s, 1.7H), 2.41 (s, 1.3H), 1.14 (t, J=7.2 Hz, 3H). Optical rotation: [a]_{D}²⁰+0.093° (c 0.10, MeOH).

### Intermediate V-3: Ethyl 4-(2-chloro-3-fluorophenyl)-6-methyl-2-(thiazol-2-yl)-1,4-dihydropyrimidine-5-carboxylate (a Mixture of 2 Stereoisomers)

By utilizing the analogous procedure of Method A, the title compound was synthesized as yellow solids.

LC-MS (ESI): R_{T}=1.74 min, mass calcd. for C₁₇H₁₅ClFN₃O₂S 379.1, m/z found 380.1 [M+H]⁺. ¹H NMR (400 MHz, CDCl₃) δ 7.84-7.80 (m, 1.7H), 7.50 (d, J=3.6 Hz, 0.3H), 7.47 (s, 0.3H), 7.44 (d, J=3.2 Hz, 0.7H), 7.23-7.14 (m, 2H), 7.09-7.01 (m, 1H), 6.27 (s, 0.7H), 6.14 (d, J=2.4 Hz, 0.3H), 4.13-3.98 (m, 2H), 2.57 (s, 0.7H), 2.52 (s, 2.3H), 1.13-1.10 (m, 3H).

The racemic mixture ethyl 4-(2-chloro-3-fluorophenyl)-6-methyl-2-(thiazol-2-yl)-1,4-dihydropyrimidine-5-carboxylate V-3 (5.45 g, 95% purity, 13.7 mmol) was separated by chiral separation (separation condition: column: Chiralpak IC 5 μm 20*250 mm; Mobile Phase: Hex:EtOH:DEA=95:5:0.3 at 28 mL/min, Temp: 30° C., Wavelength: 254 nm) to give the title compounds Va-3 (2.5 g, 90% purity from ¹H NMR, 46% yield, 100% ee) and Vb-3 (2.48 g, 90% purity from ¹H NMR, 46% yield, 92.1% ee) as yellow solids.

Va-3: LC-MS (ESI): R_{T}=3.886 min, mass calcd. for C₁₇F115ClFN₃O₂S 379.06, m/z found 380.1 [M+H]⁺. Chiral analysis (Column: Chiralpak IA 5 μm 4.6*250 mm; Mobile Phase: Hex:EtOH:DEA=90:10:0.2 at 1.0 mL/min; Temp: 30° C.; Wavelength: 254 nm, R_{T}=7.438 min). ¹H NMR (400 MHz, CDCl₃) δ 7.84-7.80 (m, 1.7H), 7.51-7.44 (m, 1.3H), 7.22-7.14 (m, 2H), 7.09-7.01 (m, 1H), 6.27 (s, 0.7H), 6.14 (s, 0.3H), 4.05-4.00 (m, 2H), 2.57 (s, 0.7H), 2.52 (s, 2.3H), 1.13-1.10 (m, 3H).

Vb-3: LC-MS (ESI): R_{T}=3.887 min, mass calcd. for C₁₇H₁₅ClFN₃O₂S 379.06, m/z found 380.1 [M+H]⁺. Chiral analysis (Column: Chiralpak IA 5 μm 4.6*250 mm; Mobile Phase: Hex:EtOH:DEA=90:10:0.2 at 1.0 mL/min; Temp: 30° C.; Wavelength: 254 nm, R_{T}=6.903 min). ¹H NMR (400 MHz, CDCl₃) δ 7.84-7.80 (m, 1.7H), 7.51-7.43 (m, 1.3H), 7.22-7.14 (m, 2H), 7.09-7.01 (m, 1H), 6.27 (s, 0.7H), 6.14 (s, 0.3H), 4.10-3.98 (m, 2H), 2.57 (s, 0.7H), 2.51 (s, 2.3H), 1.13-1.10 (m, 3H).

### Intermediate VIa-3: (R*)-Ethyl 6-(bromomethyl)-4-(2-chloro-3-fluorophenyl)-2-(thiazol-2-yl)-1,4-dihydropyrimidine-5-carboxylate (a Single Stereoisomer)

By utilizing the analogous procedure of Method B, the title compound was synthesized from Va-3 as yellow solids.

LC-MS (ESI): R_{T}=1.852 min, mass calcd. for CrHt4BrClFN₃O₂S 456.9, m/z found 457.9 [M+H]⁺. ¹H NMR (400 MHz, CDCl₃) δ 8.26 (s, 0.3H), 7.84 (d, J=2.8 Hz, 1H), 7.53-7.46 (m, 1.7H), 7.24-7.14 (m, 2H), 7.09-7.01 (m, 1H), 6.26 (s, 0.3H), 6.17 (s, 0.7H), 4.92 (d, J=8.0 Hz, 1H), 4.76 (d, J=11.2 Hz, 0.3H), 4.60 (d, J=8.0 Hz, 0.7H), 4.12 (q, J=7.2 Hz, 2H), 1.14 (t, J=11.2 Hz, 3H).

### Intermediate V-4: Methyl 4-(3-fluoro-2-methylphenyl)-6-methyl-2-(thiazol-2-yl)-1,4-dihydropyrimidine-5-carboxylate (a Mixture of 2 Stereoisomers)

By utilizing the analogous procedure of Method A, the title compound was synthesized as yellow solids.

¹H NMR (400 MHz, CDCl₃) δ 7.93 (d, J=3.2 Hz, 0.1H), 7.80-7.77 (m, 1.8H), 7.52-7.50 (m, 0.1H), 7.41 (d, J=3.2 Hz, 0.9H), 7.20 (br s, 0.1H), 7.16-7.00 (m, 2H), 6.94-6.87 (m, 1H), 6.00 (s, 0.9H), 5.90 (s, 0.1H), 3.60 (s, 3H), 2.55-2.49 (m, 5.8H), 2.40 (br s, 0.2H).

A racemic mixture of methyl 4-(3-fluoro-2-methylphenyl)-6-methyl-2-(thiazol-2-yl)-1,4-dihydropyrimidine-5-carboxylate V-4 (1.30 g, 95% purity, 3.58 mmol) was separated by chiral Prep. HPLC (separation condition: Column: Chiralpak AS-H 5 μm 30*250 mm; Mobile Phase: Hex:EtOH=75:25 at 15 mL/min; Temp: 30° C.; Wavelength: 214 nm) to afford the title compounds Vb-4 (610 mg, 95% purity from ¹H NMR, 44% yield, 100% stereopure) and Va-4 (520 mg, 95% purity from ¹H NMR, 40% yield, 97.7% stereopure) as yellow oil.

Intermediate Vb-4: Chiral analysis (Column: Chiralpak AS 5 μm 4.6*250 mm; Mobile Phase: Hex:EtOH=80:20 at 1 mL/min; Temp: 30° C.; Wavelength: 254 nm, R_{T}=5.247 min).

¹H NMR (400 MHz, CDCl₃) δ 7.93 (d, J=2.8 Hz, 0.1H), 7.80 (br s, 0.9H), 7.78 (d, J=2.8 Hz, 1H), 7.52-7.50 (m, 0.1H), 7.41 (d, J=3.2 Hz, 0.9H), 7.10-7.02 (m, 2H), 6.92-6.87 (m, 1H), 6.00 (s, 0.9H), 5.91 (s, 0.1H), 3.61 (s, 3H), 2.55 (s, 3H), 2.53 (s, 3H). Intermediate Va-4: Chiral analysis (Column: Chiralpak AS 5 μm 4.6*250 mm; Mobile Phase: Hex:EtOH=80:20 at 1 mL/min; Temp: 30° C.; Wavelength: 254 nm, R_{T}=9.049 min). ¹H NMR (400 MHz, CDCl₃) δ 7.78 (d, J=3.2 Hz, 2H), 7.42 (d, J=2.4 Hz, 1H), 7.10-7.05 (m, 2H), 6.92-6.89 (m, 1H), 5.99 (s, 1H), 3.61 (s, 3H), 2.54 (s, 3H), 2.53 (m, 3H).

### Intermediate VIa-4: (S*)-Methyl 6-(bromomethyl)-4-(3-fluoro-2-methylphenyl)-2-(thiazol-2-yl)-1,4-dihydropyrimidine-5-carboxylate (a Single Stereoisomer)

By utilizing the analogous procedure of Method B, the title compound was synthesized from Va-4 as yellow solids.

¹H NMR (400 MHz, CDCl₃) δ 8.23 (s, 1H), 7.82 (d, J=3.2 Hz, 1H), 7.53-7.44 (m, 1H), 7.12-7.07 (m, 2H), 6.93 (s, 1H), 5.98-5.94 (m, 1H), 4.89-4.66 (m, 2H), 3.65 (s, 3H), 2.53-2.41 (m, 3H).

### Intermediate V-5: Methyl 4-(2-chloro-4-fluorophenyl)-6-methyl-2-(thiazol-2-yl)-1,4-dihydropyrimidine-5-carboxylate (a Mixture of 2 Stereoisomers)

By utilizing the analogous procedure of Method A, the title compound was synthesized as yellow solids.

LC-MS (ESI): R_{T}=1.70 min, mass calcd. for C₁₆H₁₃ClFN₃O₂S 365.04, m/z found 366.1 [M+H]⁺. 1H NMR (400 MHz, CDCl₃) δ 7.84-7.83 (m, 0.9H), 7.81-7.80 (m, 0.8H), 7.55-7.50 (m, 0.6H), 7.44-7.43 (m, 0.7H), 7.33-7.26 (m, 1H), 7.13-7.11 (m, 1H), 6.95-6.88 (m, 1H), 6.18 (s, 0.7H), 6.05 (s, 0.3H), 3.63 (s, 0.8H), 3.60 (s, 2.2H), 2.57 (s, 0.8H), 2.51 (s, 2.2H).

A racemic mixture of methyl 4-(2-chloro-4-fluorophenyl)-6-methyl-2-(thiazol-2-yl)-1,4-dihydropyrimidine-5-carboxylate V-5 (20 g, 95% purity, 51.9 mmol) was separated by chiral Prep. HPLC (Column: Chiralpak IG 5 μm 30*250 mm; Mobile Phase: CO₂:MeOH=70:30 at 55 g/min; Col. Temp: 40° C.; Wavelength: 230 nm, Back pressure: 100 bar) to afford the title compounds Va-5 (9.46 g, 95% purity from NMR, 47% yield, 100% ee) and Vb-5 (9.5 g, 95% purity from NMR, 48% yield, 98.0% ee) as yellow solids.

Va-5: LC-MS (ESI): R_{T}=1.69 min, mass calcd. for C₁₆H₁₃ClFN₃O₂S 365.0, m/z found 366.0. Chiral analysis (Column: Chiralpak IA 5 μm 4.6*250 mm; Mobile Phase: Hex:EtOH=80:20 at 1.0 mL/min; Temp: 30° C.; Wavelength: 254 nm, R_{T}=5.593 min).¹H NMR (400 MHz, CDCl₃) δ 7.84-7.83 (m, 1H), 7.80 (d, J=2.8 Hz, 0.7H), 7.52-7.50 (m, 0.5H), 7.44 (d, J=2.8 Hz, 0.7H), 7.34-7.30 (m, 1H), 7.15-7.11 (m, 1H), 6.96-6.88 (m, 1H), 6.19 (s, 0.7H), 6.06 (d, J=2.4 Hz, 0.3H), 3.63 (s, 0.8H), 3.60 (s, 2.2H), 2.57 (s, 0.8H), 2.51 (s, 2.2H).

Vb-5: LC-MS (ESI): R_{T}=1.68 min, mass calcd. for C₁₆H₁₃ClFN₃O₂S 365.0, m/z found 366.0. Chiral HPLC (Column: Chiralpak IA 5 μm 4.6*250 mm; Mobile Phase: Hex:EtOH=80:20 at 1.0 mL/min; Temp: 30° C.; Wavelength: 254 nm, R_{T}=6.827 min). ¹H NMR (400 MHz, CDCl₃) 7.85-7.82 (m, 1H), 7.80 (d, J=3.2 Hz, 0.7H), 7.54-7.50 (m, 0.5H), 7.43 (d, J=3.2 Hz, 0.7H), 7.34-7.30 (m, 1H), 7.14-7.11 (m, 1H), 6.96-6.88 (m, 1H), 6.18 (s, 0.7H), 6.06 (d, J=2.4 Hz, 0.3H), 3.62 (s, 0.8H), 3.60 (s, 2.2H), 2.57 (s, 0.8H), 2.50 (s, 2.2H).

### Intermediate VIa-5: (R*)-methyl 6-(bromomethyl)-4-(2-chloro-4-fluorophenyl)-2-(thiazol-2-yl)-1,4-dihydropyrimidine-5-carboxylate (a Single Stereoisomer)

By utilizing the analogous procedure of Method B, the title compound was synthesized from Va-5 as yellow solids.

LC-MS (ESI): R_{T}=1.802 min, mass calcd. for C₁₆H₁₂BrClFN₃O₂S 442.9, m/z found 443.9 [M+H]⁺. ¹H NMR (400 MHz, CDCl₃) δ 8.29 (br s, 0.3H), 7.84 (d, J=3.2 Hz, 1H), 7.59-7.53 (m, 1.4H), 7.47 (br s, 0.3H), 7.41-7.31 (m, 1H), 7.14 (d, J=8.4 Hz, 1H), 6.99-6.90 (m, 1H), 6.18 (s, 0.3H), 6.09 (d, J=2.0 Hz, 0.7H), 4.93 (d, J=8.4 Hz, 1H), 4.74 (d, J=11.2 Hz, 0.3H), 4.58 (d, J=8.4 Hz, 0.7H), 3.67 (s, 2.1H), 3.65 (s, 0.9H).

### Intermediate V-6: Methyl 4-(2-chloro-3-fluorophenyl)-6-methyl-2-(thiazol-2-yl)-1,4-dihydropyrimidine-5-carboxylate (a Mixture of 2 Stereoisomers)

By utilizing the analogous procedure of Method A, the title compound was synthesized as yellow solids.

¹H NMR (400 MHz, CDCl₃) δ 7.86 (s, 0.8H), 7.83 (d, J=2.8 Hz, 0.3H), 7.80 (d, J=2.8 Hz, 0.7H), 7.55 (s, 0.2H), 7.50 (d, J=2.8 Hz, 0.2H), 7.44 (d, J=2.8 Hz, 0.8H), 7.23-7.13 (m, 2H), 7.11-7.00 (m, 1H), 6.25 (s, 0.8H), 6.11 (d, J=1.6 Hz, 0.2H), 3.62 (s, 0.6H), 3.60 (s, 2.4H), 2.58 (s, 0.6H), 2.51 (s, 2.4H).

A racemic mixture of methyl 4-(2-chloro-3-fluorophenyl)-6-methyl-2-(thiazol-2-yl)-1,4-dihydropyrimidine-5-carboxylate V-6 (3.00 g, 95% purity, 7.79 mmol) was separated by chiral Prep. HPLC (Column: Chiralpak IC 5 μm 20*250 mm, Mobile Phase:Hex:IPA:DEA=90:10:0.3 at 18 mL/min, Temp: 30° C., Wavelength: 230 nm) to afford the title compounds Va-6 (820 mg, 96% purity, 28% yield, 100% stereopure) and Vb-6 (800 mg, 97% purity, 27% yield, 99.2% stereopure) as yellow solids.

Compound Va-6: LC-MS (ESI): R_{T}=1.587 min, mass calcd. for C₁₆H₁₃ClFN₃O₂S 365.0, m/z found 366.0 [M+H]⁺. Chiral analysis (Column: Chiralpak IC 5 μm 4.6*250 mm; Mobile Phase: Hex:IPA:DEA=90:10:0.2 at 1 mL/min; Col. Temp: 30° C.; Wavelength: 254 nm, R_{T}=10.808 min).

¹H NMR (400 MHz, CDCl₃) δ 7.86 (s, 0.7H), 7.83 (d, J=3.2 Hz, 0.2H), 7.80 (d, J=2.8 Hz, 0.8H), 7.55 (s, 0.3H), 7.50 (d, J=3.2 Hz, 0.2H), 7.44 (d, J=3.2 Hz, 0.8H), 7.22-7.13 (m, 2H), 7.08-6.99 (m, 1H), 6.25 (s, 0.8H), 6.12 (d, J=2.4 Hz, 0.2H), 3.62 (s, 1H), 3.60 (s, 2H), 2.58 (s, 1H), 2.51 (s, 2H).

Compound Vb-6: LC-MS (ESI): R_{T}=1.584 min, mass calcd. for C₁₆H₁₃ClFN₃O₂S 365.0 m/z found 366.0 [M+H]⁺. Chiral analysis (Column: Chiralpak IC 5 μm 4.6*250 mm; Mobile Phase: Hex:IPA:DEA=90:10:0.2 at 1 Ll/min; Col. Temp: 30° C.; Wavelength: 254 nm, R_{T}=12.482 min).

¹H NMR (400 MHz, CDCl₃) δ 7.86 (s, 0.7H), 7.83 (d, J=3.2 Hz, 0.3H), 7.80 (d, J=3.2 Hz, 0.7H), 7.56 (s, 0.3H), 7.50 (d, J=2.8 Hz, 0.3H), 7.43 (d, J=3.2 Hz, 0.7H), 7.23-7.13 (m, 2H), 7.09-7.00 (m, 1H), 6.25 (s, 0.8H), 6.11 (d, J=2.0 Hz, 0.2H), 3.60 (s, 3H), 2.57 (s, 0.6H), 2.52 (s, 2.4H).

### Intermediate VIa-6: (R*)-Methyl 6-(bromomethyl)-4-(2-chloro-3-fluorophenyl)-2-(thiazol-2-yl)-1,4-dihydropyrimidine-5-carboxylate (a Single Stereoisomer)

By utilizing the analogous procedure of Method B, the title compound was synthesized from Va-6 as yellow solids.

LC-MS (ESI): R_{T}=1.695 min, mass calcd. for C₁₆H₁₂BrClFN₃O₂S 442.9 m/z found 444.0 [M+H]⁺. ¹H NMR (400 MHz, DMSO-d₆) δ 8.15-7.91 (m, 2H), 7.41-7.31 (m, 2H), 7.26-7.24 (m, 1H), 6.03 (s, 1H), 4.99-4.68 (m, 2H), 3.56 (s, 3H).

### Intermediate V-7: Methyl 4-(3,4-difluoro-2-methylphenyl)-6-methyl-2-(thiazol-2-yl)-1,4-dihydropyrimidine-5-carboxylate (a Mixture of 2 Stereoisomers)

By utilizing the analogous procedure of Method A, the title compound was synthesized as yellow solids.

LC-MS (ESI): R_{T}=1.58 min, mass calcd. for C₁₇H₁₅F₂N₃O₂S 363.3, m/z found 364.0 [M+H]⁺. ¹H NMR (400 MHz, CDCl₃) δ 7.80-7.78 (m, 2H), 7.42 (d, J=3.2 Hz, 1H), 7.00-6.85 (m, 2H), 5.93 (s, 1H), 3.61 (s, 3H), 2.58 (s, 1.5H), 2.57 (s, 1.5H), 2.53 (s, 1.5H), 2.51 (s, 1.5H).

A racemic mixture of methyl 4-(3,4-difluoro-2-methylphenyl)-6-methyl-2-(thiazol-2-yl)-1,4-dihydropyrimidine-5-carboxylate V-7 (1.00 g, 90% purity, 2.48 mmol) was separated by chiral Prep. HPLC (separation condition: Column: Chiralpak IH 5 μm 30*250 mm; Mobile Phase: Hex:EtOH=90:10 at 18 mL/min; Temp: 30° C.; Wavelength: 214 nm) to afford the desired products Va-7 (400 mg, 90% purity from ¹H NMR, 40% yield, 100% stereopure) and Vb-7 (400 mg, 95% purity from ¹H NMR, 42% yield, 99.9% stereopure) as yellow solids.

Intermediate Va-7: Chiral analysis (Column: Chiralpak IH 5 μm 4.6*150 mm; Mobile Phase: Hex:EtOH=90:10 at 1 mL/min; Temp: 30° C.; Wavelength: 230 nm, R_{T}=4.809 min). ¹H NMR (400 MHz, CDCl₃) δ 7.84 (br s, 1H), 7.78 (d, J=3.2 Hz, 1H), 7.42 (d, J=3.2 Hz, 1H), 6.96-6.86 (m, 2H), 5.93 (s, 1H), 3.61 (s, 3H), 2.57 (d, J=1.6 Hz, 3H), 2.52 (s, 3H).

Intermediate Vb-7: Chiral analysis (Column: Chiralpak IH 5 μm 4.6*150 mm; Mobile Phase: Hex:EtOH=90:10 at 1 mL/min; Temp: 30° C.; Wavelength: 230 nm, R_{T}=7.018 min). ¹H NMR (400 MHz, CDCl₃) δ 7.82 (br s, 1H), 7.79 (d, J=3.2 Hz, 1H), 7.42 (d, J=3.2 Hz, 1H), 6.97-6.88 (m, 2H), 5.93 (s, 1H), 3.61 (s, 3H), 2.58 (d, J=2.0 Hz, 3H), 2.52 (s, 3H).

### Intermediate VIb-7: (S*)-Methyl 6-(bromomethyl)-4-(3,4-difluoro-2-methylphenyl)-2-(thiazol-2-yl)-1,4-dihydropyrimidine-5-carboxylate (a Single Stereoisomer)

By utilizing the analogous procedure of Method B, the title compound was synthesized from Vb-7 as yellow solids.

¹H NMR (400 MHz, CDCl₃) δ 8.24 (s, 1H), 7.83 (d, J=3.6 Hz, 1H), 7.54-7.45 (m, 1H), 7.00-6.93 (m, 2H), 5.91 (s, 1H), 4.94-4.80 (s, 21H), 3.66 (s, 3H), 2.56-2.45 (m, 3H).

### Intermediate V-8: Methyl 4-(2-chloro-3,4-difluorophenyl)-6-methyl-2-(thiazol-2-yl)-1,4-dihydropyrimidine-5-carboxylate (a Mixture of 2 Stereoisomers)

By utilizing the analogous procedure of Method A, the title compound was synthesized as yellow solids.

¹H NMR (400 MHz, CD₃OD) δ 8.08 (d, J=2.8 Hz, 0.1H), 7.98 (d, J=2.8 Hz, 0.1H), 7.93 (d, J=2.8 Hz, 0.9H), 7.72 (d, J=2.8 Hz, 0.9H), 7.26-7.18 (m, 2H), 6.13 (s, 0.9H), 6.09 (s, 0.1H), 3.61 (s, 3H), 2.53 (s, 3H).

Racemic V-8 (1.10 g, 2.90 mmol) was separated by chiral Prep-HPLC (separation condition: Column: Chiralpak IC 5 μm 20*250 mm; Mobile Phase: Hex:EtOH=90:10 at 18 mL/min; Temp: 30° C.; Wavelength: 214 nm) to afford the title compounds Va-8 (450 mg, 41% yield, 100% stereopure) and Vb-8 (450 m g, 41% yield, 99.8% stereopure) as yellow solids.

Intermediate Va-8: Chiral analysis (Column: Chiralpak IC 5 μm 4.6*250 mm; Mobile Phase: Hex:EtOH=90:10 at 1.0 mL/min; Temp: 30° C.; Wavelength: 254 nm, R_{T}=6.457 min).

Intermediate Vb-8: Chiral analysis (Column: Chiralpak IC 5 μm 4.6*250 mm; Mobile Phase: Hex:EtOH=90:10 at 1.0 mL/min; Temp: 30° C.; Wavelength: 254 nm, R_{T}=7.641 min).

### Intermediate VIa-8: (R*)-methyl 6-(bromomethyl)-4-(2-chloro-3,4-difluorophenyl)-2-(thiazol-2-yl)-1,4-dihydropyrimidine-5-carboxylate (a Single Stereoisomer)

By utilizing the analogous procedure of Method B, the title compound was synthesized from Va-8 as yellow solids.

¹H NMR (400 MHz, CD₃OD) δ 7.92 (d, J=3.2 Hz, 1H), 7.80 (d, J=3.2 Hz, 0.5H), 7.70 (d, J=3.2 Hz, 0.5H), 7.32-7.17 (m, 2H), 6.11 (s, 0.5H), 6.09 (s, 0.5H), 4.91 (d, J=10.0 Hz, 0.5H), 4.81 (d, J=10.0 Hz, 1H), 4.57 (d, J=8.4 Hz, 0.5H), 3.64 (s, 1.5H), 3.62 (s, 1.5H).

### Intermediate V-9: Ethyl 4-(2-chloro-3,4-difluorophenyl)-6-methyl-2-(thiazol-2-yl)-1,4-dihydropyrimidine-5-carboxylate (a Mixture of 2 Stereoisomers)

By utilizing the analogous procedure of Method A, the title compound was synthesized as yellow solids.

¹H NMR (400 MHz, CDCl₃) δ7.83-7.81 (m, 1.8H), 7.52-7.44 (m, 1.2H), 7.13-7.10 (m, 1H), 7.08-7.00 (m, 1H), 6.20 (s, 0.8H), 6.08 (s, 0.2H), 4.11-4.00 (m, 2H), 2.57 (s, 0.5H), 2.51 (s, 2.5H), 1.13 (t, J=7.2 Hz, 3H).

Racemic V-9 (1.00 g, 2.51 mmol) was separated by chiral Prep-HPLC (Column: Chiralpak IC 5 μm 20*250 mm; Mobile Phase: Hex:EtOH=90:10 at 18 mL/min; Temp: 30° C.; Wavelength: 214 nm) to give the desired compound Va-9 (353 mg, 35% yield, 98.1% stereopure) and Vb-9 (321 mg, 32% yield, 99.8% stereopure) as yellow solids.

Intermediate Va-9: Chiral analysis (Column: Chiralpak IC 5 μm 4.6*250 mm; Mobile Phase: Hex:EtOH=90:10 at 1.0 mL/min; Temp: 30° C.; Wavelength: 254 nm, R_{T}=5.901 min).

Intermediate Vb-9: Chiral analysis (Column: Chiralpak IC 5 μm 4.6*250 mm; Mobile Phase: Hex:EtOH=90:10 at 1.0 mL/min; Temp: 30° C.; Wavelength: 254 nm, R_{T}=6.914 min).

### Intermediate VIa-9: (R*)-Ethyl 6-(bromomethyl)-4-(2-chloro-3,4-difluorophenyl)-2-(thiazol-2-yl)-1,4-dihydropyrimidine-5-carboxylate (a Single Stereoisomer)

By utilizing the analogous procedure of Method B, the title compound was synthesized from Va-9 as yellow solids.

¹H NMR (400 MHz, CDCl₃) δ 8.25 (s, 0.3H), 7.85 (d, J=3.2 Hz, 1H), 7.54 (d, J=3.2 Hz, 0.6H), 7.47-7.45 (m, 0.9H), 7.22-7.00 (m, 2.2H), 6.19 (s, 0.4H), 6.11 (d, J=2.4 Hz, 0.6H), 4.97 (d, J=11.2 Hz, 0.4H), 4.94 (d, J=8.8 Hz, 0.6H), 4.73 (d, J=11.2 Hz, 0.4H), 4.56 (d, J=8.4 Hz, 0.6H), 4.16-4.04 (m, 2H), 1.19-1.13 (m, 3H).

### Intermediate V-10: methyl 4-(2-chloro-4-fluorophenyl)-2-(3,5-difluoropyridin-2-yl)-6-methyl-1,4-dihydropyrimidine-5-carboxylate (a Mixture of 2 Stereoisomers)

By utilizing the analogous procedure of Method A, the title compound was synthesized as yellow solids.

LC-MS (ESI): R_{T}=1.657 min, mass calcd. for C18H₁₃ClF₃N₃O₂ 395.1, m/z found 396.1 [M+H]⁺. ¹H NMR (400 MHz, CD₃OD) δ 8.43 (d, J=2.4 Hz, 1H), 7.71-7.65 (m, 1H), 7.45 (dd, J=8.4 Hz, 6.4 Hz, 1H), 7.21 (dd, J=8.8 Hz, 2.4 Hz, 1H), 7.04 (dt, J=8.4 Hz, 2.4 Hz, 1H), 6.16 (s, 1H), 3.59 (s, 3H), 2.48 (s, 3H).

### Intermediates Va-10 and Vb-10: (R*)-methyl 4-(2-chloro-4-fluorophenyl)-2-(3,5-difluoropyridin-2-yl)-6-methyl-1,4-dihydropyrimidine-5-carboxylate (a Single Stereoisomer) and

### (S*)-methyl 4-(2-chloro-4-fluorophenyl)-2-(3,5-difluoropyridin-2-yl)-6-methyl-1,4-dihydropyrimidine-5-carboxylate (a Single Stereoisomer)

A racemic mixture of methyl 4-(2-chloro-4-fluorophenyl)-2-(3,5-difluoropyridin-2-yl)-6-methyl-1,4-dihydropyrimidine-5-carboxylate V-10 (1.2 g, 95% purity, 2.88 mmol) was separated by chiral Prep. HPLC (separation condition: Column: Chiralpak AS-H 5 μm 20*250 mm; Mobile Phase: Hex:EtOH=80:20 at 18 mL/min; Temp: 40° C.; Wavelength: 214 nm) to give Va-10 (550 mg, 95% purity from ¹H NMR, 46% yield, 100% stereopure) and Vb-10 (530 mg, 95% purity from ¹H NMR, 44% yield, 99.8% stereopure) as yellow solids.

Intermediate Va-10: LC-MS (ESI): R_{T}=1.692 min, mass calcd. for C₁₈H₁₃ClF₃N₃O₂ 395.1, m/z found 396.1 [M+H]⁺. Chiral analysis (Column: Chiralpak AS-H, 5 μm 4.6*250 mm; Mobile Phase: Hex:EtOH=80:20 at 1 mL/min; Temp: 30° C.; Wavelength: 254 nm; R_{T}=4.952 min). ¹H NMR (400 MHz, CD₃OD) δ 8.43 (s, 1H), 7.70-7.66 (m, 1H), 7.46-7.43 (m, 1H), 7.19 (d, J=7.2 Hz, 1H), 7.04 (t, J=8.0 Hz, 1H), 6.16 (s, 1H), 3.59 (s, 3H), 2.48 (s, 3H).

Intermediate Vb-10: LC-MS (ESI): R_{T}=1.692 min, mass calcd. for C₁₈H₁₃ClF₃N₃O₂ 395.1, m/z found 396.1 [M+H]⁺. Chiral analysis (Column: Chiralpak AS-H, 5 μm 4.6*250 mm; Mobile Phase: Hex:EtOH=80:20 at 1 mL/min; Temp: 30° C.; Wavelength: 254 nm; R_{T}=7.598 min). ¹H NMR (400 MHz, CD₃OD) δ 8.43 (d, J=2.4 Hz, 1H), 7.71-7.65 (m, 1H), 7.45 (dd, J=8.4 Hz, 6.4 Hz, 1H), 7.21 (dd, J=8.8 Hz, 2.4 Hz, 1H), 7.04 (dt, J=8.4 Hz, 2.4 Hz, 1H), 6.16 (s, 1H), 3.59 (s, 3H), 2.48 (s, 3H).

### Intermediate VIa-10: (R*)-methyl 6-(bromomethyl)-4-(2-chloro-4-fluorophenyl)-2-(3,5-difluoropyridin-2-yl)-1,4-dihydropyrimidine-5-carboxylate (a Single Stereoisomer)

By utilizing the analogous procedure of Method B, the title compound was synthesized from Va-10 as yellow solids.

LC-MS (ESI): R_{T}=1.808 min, mass calcd. for C₁₅H₁₂BrClF₃N₃O₂ 473.0, m/z found 474.0 [M+H]⁺. ¹H NMR (400 MHz, CD₃OD) δ 8.47 (d, J=2.4 Hz, 1H), 7.75-7.70 (m, 1H), 7.54 (dd, J=8.8 Hz, 6.4 Hz, 1H), 7.28 (dd, J=8.8 Hz, 2.8 Hz, 1H), 7.12-7.07 (m, 1H), 6.19 (s, 1H), 4.92 (d, J=9.6 Hz, 1H), 4.75 (d, J=9.6 Hz, 1H), 3.68 (s, 3H).

### Intermediate VIb-10: (S*)-methyl 6-(bromomethyl)-4-(2-chloro-4-fluorophenyl)-2-(3,5-difluoropyridin-2-yl)-1,4-dihydropyrimidine-5-carboxylate (a Single Stereoisomer)

By utilizing the analogous procedure of Method B, the title compound was synthesized from Vb-10 as yellow solids.

LC-MS (ESI): R_{T}=1.802 min, mass calcd. for C₁₈H₁₂BrClF₃N₃O₂ 473.0, m/z found 474.0 [M+H]⁺. ¹H NMR (400 MHz, CD₃OD) δ 8.47 (d, J=2.4 Hz, 1H), 7.75-7.70 (m, 1H), 7.55 (dd, J=10.0 Hz, 8.8 Hz, 1H), 7.28 (dd, J=8.8 Hz, 2.8 Hz, 1H), 7.12-7.07 (m, 1H), 6.19 (s, 1H), 4.92 (d, J=9.6 Hz, 1H), 4.75 (d, J=9.2 Hz, 1H), 3.68 (s, 3H).

### Compound I-1A: 3-((4aR*,7aS*)-4-4(R*)-6-(2-chloro-4-fluorophenyl)-5-(ethoxycarbonyl)-2-(thiazol-2-yl)-3,6-dihydropyrimidin-4-yl)methyphexahydropyrrolo[3,4-b][1,4]oxazin-6(2H)-yl)-2,2-dimethylpropanoic acid (a Single Stereoisomer)

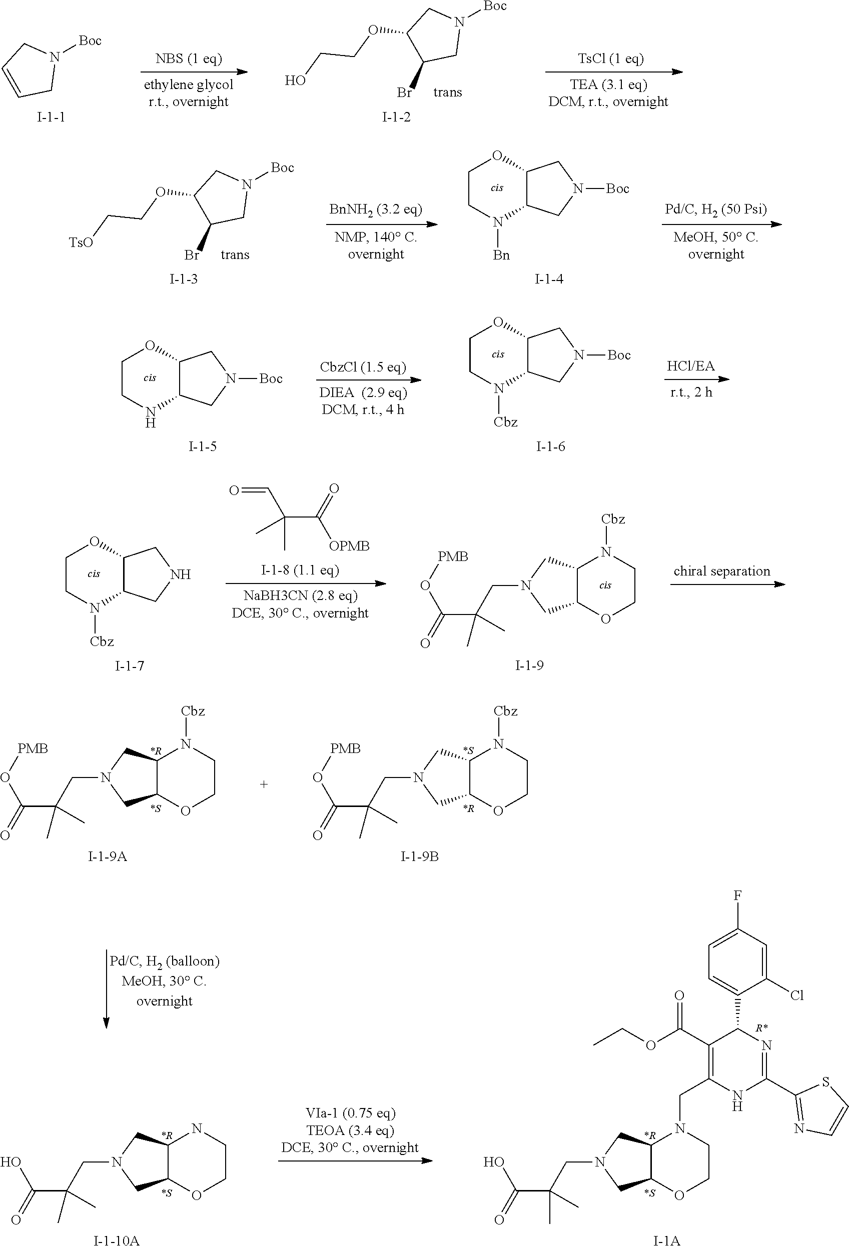

### Intermediate I-1-2: (trans)-tert-butyl 3-bromo-4-(2-hydroxyethoxy)pyrrolidine-1-carboxylate (a Mixture of 2 Stereoisomers)

To a solution of tert-butyl 2,5-dihydro-1H-pyrrole-1-carboxylate I-1-1(23 g, 136 mmol) in ethylene glycol (100 mL) was added N-bromosuccinimide (25 g, 140 mmol) under nitrogen at 0° C. After stirred at room temperature under nitrogen atmosphere overnight, the mixture was poured into water (200 mL) and extracted with ethyl acetate (300 mL) for three times. The combined organic layers were washed with brine (300 mL), dried over Na₂SO₄(s) and filtered. The filtrate was concentrated and purified by silica gel column chromatography (petroleum ether:ethyl acetate=3:1) to give the title compound (35 g, 90% purity from ¹H NMR, 70% yield) as colorless oil.

¹H NMR (400 MHz, CDCl₃) δ 4.30 (s, 1H), 4.16-4.15 (m, 1H), 3.96-3.92 (m, 1H), 3.87-3.61 (m, 6H), 3.52-3.43 (m, 1H), 2.05 (br s, 1H), 1.47 (s, 9H).

### Intermediate I-1-3: (trans)-tert-butyl 3-bromo-4-(2-(tosyloxy)ethoxy)pyrrolidine-1-carboxylate (a Mixture of 2 Stereoisomers)

To a solution of (trans)-tert-butyl 3-bromo-4-(2-hydroxyethoxy)pyrrolidine-1-carboxylate I-1-2 (26.3 g, 90% purity, 76.3 mmol) and triethylamine (24 g, 237 mmol) in dichloromethane (150 mL) was added tosyl chloride (15 g, 78.7 mmol) at 0° C. After stirred at room temperature overnight, the mixture was poured into water (200 mL) and extracted with ethyl acetate (300 mL) for three times. The combined organic layers were washed with brine (300 mL), dried over Na₂SO₄₍ₛ₎ and filtered. The filtrate was concentrated and purified by silica gel column chromatography (petroleum ether:ethyl acetate=5:1) to give the title compound (30 g, 90% purity from ¹H NMR, 76% yield) as colorless oil.

¹H NMR (400 MHz, CDCl₃) δ 7.79 (d, J=8.0 Hz, 2H), 7.35 (d, J=8.0 Hz, 2H), 4.15-4.11 (m, 3H), 4.08-4.04 (m, 1H), 3.86-3.68 (m, 5H), 3.35 (t, J=12.0 Hz, 1H), 2.46 (s, 3H), 1.47 (s, 9H).

### Intermediate I-1-4: (cis)-tert-butyl 4-benzylhexahydropyrrolo[3,4-b][1,4]oxazine-6(2H)-carboxylate (a Mixture of 2 Stereoisomers)

The mixture of (trans)-tert-butyl 3-bromo-4-(2-(tosyloxy)ethoxy)pyrrolidine-1-carboxylate I-1-3 (30 g, 90% purity, 58.1 mmol) and benzylamine (20 g, 187 mmol) in 1-methyl-2-pyrrolidinone (100 mL) was stirred at 140° C. overnight. After cooled down to room temperature, the mixture was poured into water (500 mL) and extracted with ethyl acetate (300 mL) for three times. The combined organic layers were washed with water (500 mL) for three times, dried over Na₂SO₄(s) and filtered. The filtrate was concentrated and purified by silica gel column chromatography (petroleum ether:ethyl acetate=5:1) to give the title compound (15.7 g, 90% purity from ¹H NMR, 76% yield) as yellow oil.

¹H NMR (400 MHz, CDCl₃) δ 7.35-7.27 (m, 5H), 4.04-3.99 (m, 1H), 3.87-3.82 (m, 1H), 3.70-3.57 (m, 4H), 3.46-3.22 (m, 4H), 2.78-2.68 (m, 1H), 2.49-2.44 (m, 1H), 1.47-1.44 (m, 9H).

### Intermediate I-1-5: (cis)-tert-butyl hexahydropyrrolo[3,4-b][1,4]oxazine-6(2H)-carboxylate (a Mixture of 2 Stereoisomers)

To the solution of (cis)-tert-butyl 4-benzylhexahydropyrrolo[3,4-b][1,4]oxazine-6(2H)-carboxylate I-1-4 (15.7 g, 90% purity, 44.37 mmol) in methanol (100 mL) was added 10% palladium on charcoal wt. (5.0 g). After stirred at 50° C. under hydrogen atmosphere (50 psi) overnight, the mixture was filtered. The filtrate was concentrated to give the title compound (10.0 g, 90% purity from ¹H NMR, 89% yield) as colorless oil.

¹H NMR (400 MHz, CDCl₃) δ 3.96 (s, 1H), 3.84 (dt, J=11.2 Hz, 2.4 Hz, 1H), 3.58-3.52 (m, 2H), 3.48-3.36 (m, 4H), 3.16-3.09 (m, 1H), 2.67 (dt, J=13.2 Hz, 2.4 Hz, 1H), 1.45 (s, 9H).

### Intermediate I-1-6: (cis)-4-benzyl 6-tert-butyl hexahydropyrrolo[3,4-b][1,4]oxazine-4,6-dicarboxylate (a Mixture of 2 Stereoisomers)

To a solution of (cis)-tert-butyl hexahydropyrrolo[3,4-b][1,4]oxazine-6(2H)-carboxylate I-1-5 (10 g, 90% purity, 39.4 mmol), and N,N-diisopropyl-ethylamine (15.0 g, 116 mmol) in dichloromethane (100 mL) was benzyl carbonochloridate (10.0 g, 170 mmol) at 0° C. After stirred at room temperature for 4 hours, the mixture was poured into water (100 mL) and extracted with dichloromethane (200 mL) for three times. The combined organic layers were washed with brine (500 mL), dried over Na₂SO₄₍ₛ₎ and filtered. The filtrate was concentrated and purified by silica gel column chromatography (petroleum ether:ethyl acetate=5:1) to give the title compound (15.4 g, 90% purity from ¹H NMR, 97% yield) as yellow oil.

¹H NMR (300 MHz, CDCl₃) δ 7.42 (s, 5H), 5.21 (s, 2H), 4.64-4.44 (m, 1H), 4.02-3.06 (m, 9H), 1.51 (s, 9H).

### Intermediates I-1-7: (cis)-benzylhexahydropyrrolo[3,4-b][1,4]oxazine-4(4aH)-carboxylate (a Mixture of 2 Stereoisomers)

The solution of (cis)-4-benzyl 6-tert-butyl hexahydropyrrolo[3,4-b][1,4]oxazine-4,6-dicarboxylate I-1-6 (5.1 g, 90% purity, 12.7 mmol) in 2 M hydrochloride in ethyl acetate (20 mL) was stirred at room temperature for 2 hours. The mixture was poured into saturated sodium carbonate aqueous solution (20 mL) and extracted with ethyl acetate (20 mL) for three times. The combined organic layers were washed with brine (50 mL), dried over Na₂SO₄(s) and filtered. The filtrate was concentrated to give title compound (3.3 g, 90% purity from ¹H NMR, 89% yield) as yellow oil.

¹H NMR (400 MHz, CDCl₃) δ 7.39-7.30 (m, 5H), 5.19-5.11 (m, 2H), 4.38-4.30 (m, 1H), 3.90-3.46 (m, 5H), 3.26-2.87 (m, 4H).

### Intermediate I-1-9: (cis)-benzyl 6-(3-((4-methoxybenzyl)oxy)-2,2-dimethyl-3-oxopropyl)hexahydropyrrolo[3,4-b][1,4]oxazine-4(4aH)-carboxylate (a Mixture of 2 Stereoisomers)

The solution of (cis)-benzylhexahydropyrrolo[3,4-b][1,4]oxazine-4(4aH)-carboxylate I-1-7 (1.0 g, 90% purity, 3.43 mmol) and 4-methoxybenzyl 2,2-dimethyl-3-oxopropanoate I-1-8 (1.0 g, 3.81 mmol, 90% purity) in 1,2-dichloroethane (10 mL) was stirred at 30° C. for 0.5 hour before sodium cyanoborohydride (600 mg, 9.55 mmol) was added. Afetr stirred at 30° C. overnight, the mixture was poured into water (20 mL) and extracted with dichloromethane (30 mL) for three times. The combined organic layers were washed with brine (30 mL), dried over Na₂SO₄₍ₛ₎ and filtered. The filtrate was concentrated and purified by C18 column (acetonitrile:water=30% to 60%) to give the title compound (600 mg, 90% purity from ¹H NMR, 32% yield).

¹H NMR (400 MHz, CDCl₃) δ 7.38-7.23 (m, 7H), 6.89-6.83 (m, 2H), 5.18-5.00 (m, 4H), 4.35-4.17 (m, 1H), 3.86-3.69 (m, 6H), 3.47-3.40 (m, 1H), 3.26-3.20 (m, 1H), 3.16-2.53 (m, 6H), 1.15 (s, 6H).

### Intermediates I-1-9A and I-1-9B: (4aR*,7aS*)-benzyl 6-(3-((4-methoxybenzyl)oxy)-2,2-dimethyl-3-oxopropyl)hexahydropyrrolo[3,4-b][1,4]oxazine-4(4aH)-carboxylate (a Single Stereoisomer) and

### (4aS*,7aR*)-benzyl 6-(3-((4-methoxybenzyl)oxy)-2,2-dimethyl-3-oxopropyl)hexahydropyrrolo[3,4-b][1,4]oxazine-4(4aH)-carboxylate (a Single Stereoisomer)

A mixture of (cis)-benzyl 6-(3-((4-methoxybenzyl)oxy)-2,2-dimethyl-3-oxopropyl) hexahydropyrrolo[3,4-b][1,4]oxazine-4(4aH)-carboxylate I-1-9 (3 g, 90% purity, 5.60 mmol) was separated by chiral Prep. HPLC (Column: Chiralpak IF 5 μm 20*250 mm; Mobile Phase: Hex:EtOH=70:30 at 18 mL/min; Temp: 30° C.; Wavelength: 214 nm) to give the title compounds I-1-9A (1.2 g, 95% purity from ¹H NMR, 42% yield, 100% stereopure) and I-1-9B (1.1 g, 95% purity from ¹H NMR, 38% yield, 97.6% stereopure) as yellow oil.

Intermediate I-1-9A: Chiral analysis (Column: Chiralpak IF 5 μm 4.6*250 mm; Mobile Phase: Hex:EtOH=70:30 at 1 mL/min; Temp: 30° C.; Wavelength: 254 nm; R_{T}=8.127 min). ¹H NMR (400 MHz, CDCl₃) δ 7.38-7.23 (m, 7H), 6.89-6.83 (m, 2H), 5.15-5.00 (m, 4H), 4.32-4.20 (m, 1H), 3.87-3.69 (m, 6H), 3.47-3.42 (m, 1H), 3.26-3.22 (m, 1H), 3.13-2.53 (m, 6H), 1.15 (s, 6H).

Intermediate I-1-9B: Chiral analysis (Column: Chiralpak IF 5 μm 4.6*250 mm; Mobile Phase: Hex:EtOH=70:30 at 1 mL/min; Temp: 30° C.; Wavelength: 254 nm; R_{T}=9.391 min). ¹H NMR (400 MHz, CDCl₃) δ 7.38-7.23 (m, 7H), 6.89-6.83 (m, 2H), 5.18-5.00 (m, 4H), 4.32-4.18 (m, 1H), 3.86-3.79 (m, 6H), 3.47-3.40 (m, 1H), 3.26-3.22 (m, 1H), 3.16-3.28 (m, 2H), 2.81-2.53 (m, 4H), 1.15 (s, 6H).

### Intermediate: 3-((4aR*,7aS*)-hexahydropyrrolo[3,4-b][1,4]oxazin-6(2H)-yl)-2,2-dimethylpropanoic acid (a Single Stereoisomer)

A mixture of (4aR*,7aS*)-benzyl 6-(3-((4-methoxybenzyl)oxy)-2,2-dimethyl-3-oxopropyl)hexahydropyrrolo[3,4-b][1,4]oxazine-4(4aH)-carboxylate I-1-9A (1.2 g, 95% purity, 2.36 mmol) and 10% wt. palladium on charcoal (600 mg) in methanol (20 mL) was stirred at 30° C. under hydrogen balloon overnight. The mixture was filtered and the filtrate was concentrated to give a crude title compound (477 mg, 95% purity from ¹H NMR, 84% yield) as yellow solids. crude title compound (200 mg, 95% purity) was triturated with a solution of methanol (5 drops) and acetonitrile (5 mL). The yellow solids was collected by filtration to give pure title compound (100 mg, 99.4% purity, 18% yield) as yellow solids.

LC-MS (ESI): R_{T}=7.443 min, mass calcd. for C₁₁H₂₀N₂O₃ 228.1, m/z found 229.1 [M+H]⁺. ¹H NMR (400 MHz, CDCl₃) δ 3.99-3.92 (m, 1H), 3.84-3.81 (m, 1H), 3.55-3.46 (m, 2H), 3.32-3.19 (m, 2H), 3.12-2.98 (m, 2H), 2.94-2.84 (m, 2H), 2.79-2.67 (m, 2H), 1.23 (s, 3H), 1.21 (s, 3H).

### Compound I-1A: 3-((4aR*,7aS*)-4-4(R*)-6-(2-chloro-4-fluorophenyl)-5-(ethoxycarbonyl)-2-(thiazol-2-yl)-3,6-dihydropyrimidin-4-yl)methyl)hexahydropyrrolo[3,4-b][1,4]oxazin-6(2H)-yl)-2,2-dimethylpropanoic acid (a Single Stereoisomer)

To a solution of 3-((4aR*,7aS*)-hexahydropyrrolo[3,4-b][1,4]oxazin-6(2H)-yl)-2,2-dimethylpropanoic acid I-1-10A (70 mg, 90% purity, 0.276 mmol) and triethanolamine (100 mg, 0.934 mmol) in 1,2-dichloroethane (5 mL) was added (R*)-ethyl 6-(bromomethyl)-4-(2-chloro-4-fluorophenyl)-2-(thiazol-2-yl)-1,4-dihydropyrimidine-5-carboxylate VIa-1 (100 mg, 95% purity, 0.207 mmol). After stirred at 30° C. overnight under nitrogen atmosphere, the mixture was quenched with water (10 mL) and extracted with dichloromethane (20 mL) for three times. Then combined organic layers were washed with brine (20 mL), dried over Na₂SO₄₍ₛ₎ and filtered. The filtrate was concentrated and purified by C18 column (acetonitrile:water=5% to 50%) to give the title compound (70 mg, 97.8% purity, 40% yield) as yellow solids.

LC-MS (ESI): R_{T}=3.811 min, mass calcd. for C₂₈H₃₃ClFN₅O₅S 605.2, m/z found 606.2 [M+H]⁺. ¹H NMR (400 MHz, DMSO-d₆) δ 13.13 (br s, 1H), 9.63 (s, 1H), 8.01 (d, J=3.2 Hz, 1H), 7.94 (d, J=3.2 Hz, 1H), 7.43-7.38 (m, 2H), 7.20-7.15 (m, 1H), 6.05 (s, 1H), 4.07-3.93 (m, 5H), 3.86-3.83 (m, 1H), 3.59-3.53 (m, 1H), 3.14-3.05 (m, 2H), 2.81-2.67 (m, 5H), 2.56-2.51 (m, 2H), 1.07-1.02 (m, 9H).

### Compound I-2A: 4-((4aR*,7aS*)-4-4(S)-5-(ethoxycarbonyl)-6-(3-fluoro-2-methylphenyl)-2-(thiazol-2-yl)-3,6-dihydropyrimidin-4-yl)methyphexahydropyrrolo[3,4-b][1,4]oxazin-6(2H)-yl)-2,2-dimethyl-4-oxobutanoic acid (a Single Stereoisomer)

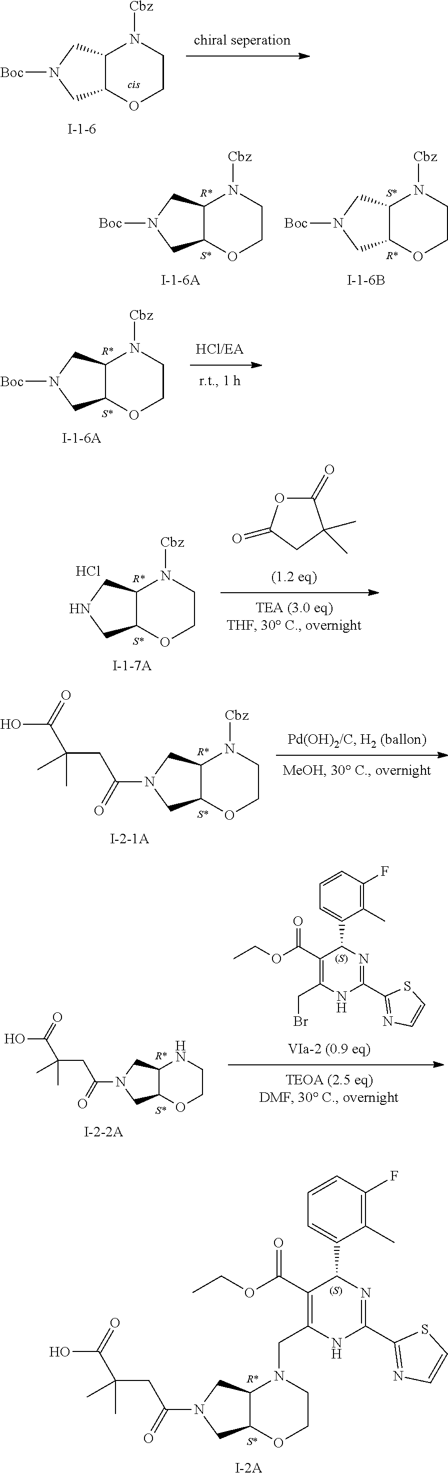

### Intermediates I-1-6A and I-1-6B: (4aR*,7aS*)-4-benzyl 6-tert-butyl hexahydropyrrolo[3,4-b][1,4]oxazine-4,6-dicarboxylate (a Single Stereoisomer) and (4aS*,7aR*)-4-benzyl 6-tert-butyl hexahydropyrrolo[3,4-b][1,4]oxazine-4,6-dicarboxylate (a Single Stereoisomer)

A mixture of (cis)-4-benzyl 6-tert-butyl hexahydropyrrolo[3,4-b][1,4]oxazine-4,6-dicarboxylate I-1-6 (8.0 g, 20.97 mmol) was separated by chiral Prep. HPLC (separation condition: Column: Chiralpak IC 5 um 20*250 mm; Mobile Phase: CO₂:IPA=75:25 at 50 g/min; Temp: 40° C.; Wavelength: 214 nm) to give I-1-6A (4.0 g, 95% purity, 50% yield, 100% stereopure) and I-1-6B (4.0 g, 95% purity, 50% yield, 99.4% stereopure) as yellow oil.

Intermediate I-1-6A: LC-MS (ESI): R_{T}=3.731 min, mass calcd. for C₁₉H₂₆N205 362.2, m/z found 263.1 [M+H-100]⁺. Chiral analysis (Column: Chiralpak IC, 5 μm 4.6*250 mm; Mobile Phase: CO₂:IPA=75:25 at 3.0 g/min; Temp: 40° C.; Wavelength: 214 nm; Back pressure: 100 bar, R_{T}=4.31 min).

Intermediate I-1-6B: LC-MS (ESI): R_{T}=3.727 min, mass calcd. for C₁₉H₂₆N₂O₅ 362.2, m/z found 263.2 [M+H-100]⁺. Chiral analysis (Column: Chiralpak IC, 5 μm 4.6*250 mm; Mobile Phase: CO₂:IPA=75:25 at 3.0 g/min; Temp: 40° C.; Wavelength: 214 nm; Back pressure: 100 bar, R_{T}=7.08 min).

### Intermediate I-1-7A: (4aR*,7aS*)-benzyl hexahydropyrrolo[3,4-b][1,4]oxazine-4(4aH)-carboxylate hydrochloride (a Single Stereoisomer)

The solution of (4aR*,7aS*)-4-benzyl 6-tert-butyl hexahydropyrrolo[3,4-b][1,4]oxazine-4,6-dicarboxylate I-1-6A (400 mg, 95% purity, 1.05 mmol) in 4 M hydrochloride in ethyl acetate (16 mL) was stirred at room temperature under nitrogen atmosphere for 1 hour. Then the mixture was concentrated to give the title compound (340 mg, 90% purity from ¹H NMR, 98% yield) as white solids. LC-MS (ESI): R_{T}=1.30 min, mass calcd. for C₁₄H₁₈N₂O₃ 262.1, m/z found 263.0 [M+H]⁺.

¹H NMR (400 MHz, D₂O) δ 7.48 (s, 5H), 5.23 (s, 2H), 4.92-4.69 (m, 1H), 4.25 (s, 1H), 4.01-3.79 (m, 2H), 3.67-3.52 (m, 4H), 3.42-3.36 (m, 1H), 3.30-3.17 (m, 1H).

### Intermediate I-2-1A: 4-((4aR*,7aS*)-4-((benzyloxy)carbonyl)hexahydropyrrolo[3,4-b][1,4]oxazin-6(2H)-yl)-2,2-dimethyl-4-oxobutanoic acid (a Single Stereoisomer)

To a solution of (4aR*,7aS*)-benzyl hexahydropyrrolo[3,4-b][1,4]oxazine-4(4aH)-carboxylate hydrochloride I-1-7A (340 mg, 90% purity, 1.02 mmol) in tetrahydrofuran (10 mL) was added 3,3-dimethyldihydrofuran-2,5-dione (160 mg, 1.25 mmol) and triethylamine (310 mg, 3.06 mmol) under nitrogen atmosphere. After stirred at 30° C. overnight, the mixture was diluted with ethyl acetate (20 mL) and washed with water (40 mL). The aqueous layer was extracted with ethyl acetate (10 mL) for three times. Then the combined organic layers were dried over Na₂SO₄₍ₛ₎ and filtered. The filtrate was and concentrated and purified by C18 column (acetonitrile:water=50% to 85%) to give the title compound (340 mg, 95% purity from ¹H NMR, 81% yield) as yellow oil.

LC-MS (ESI): R_{T}=1.31 min, mass calcd. for C₂₀H₂₆N₂O₆ 390.2, m/z found 391.1 [M+H]⁺. ¹H NMR (400 MHz, CDCl₃) δ 7.37-7.33 (m, 5H), 5.22-5.11 (m, 2H), 4.62-4.58 (m, 0.5H), 4.52-4.45 (m, 0.5H), 4.04-3.39 (m, 8H), 3.22-3.11 (m, 1H), 2.63-2.43 (m, 2H), 1.31-1.30 (m, 6H).

### Intermediate I-2-2A: 4-((4aR*,7aS*)-hexahydropyrrolo[3,4-b][1,4]oxazin-6(2H)-yl)-2,2-dimethyl-4-oxobutanoic acid (a Single Stereoisomer)

To a solution of 4-((4aR*,7aS*)-4-((benzyloxy)carbonyl)hexahydropyrrolo[3,4-b][1,4]oxazin-6(2H)-yl)-2,2-dimethyl-4-oxobutanoic acid I-2-1A (100 mg, 95% purity, 0.243 mmol) in methanol (20 mL) was added 20% palladium hydroxide on charcoal wt. (30 mg) under nitrogen atmosphere. After stirred at 30° C. under hydrogen balloon overnight, the mixture was cooled down to room temperature and filtered. The filtrate was concentrated to give the title compound (50 mg, 90% purity from ¹H NMR, 72% yield) as yellow oil which was directly used in next step without further purification.

¹H NMR (400 MHz, CDCl₃) δ 4.30 (s, 0.4H), 4.15 (s, 0.6H), 3.90-3.87 (m, 1H), 3.77-3.31 (m, 6H), 3.22-3.13 (m, 0.5H), 3.01-2.93 (m, 0.5H), 2.74-2.61 (m, 1H), 2.52-2.39 (m, 2H), 1.29-1.23 (m, 6H).

### Compound I-2A: 4-((4aR*,7aS*)-4-4(S)-5-(ethoxycarbonyl)-6-(3-fluoro-2-methylphenyl)-2-(thiazol-2-yl)-3,6-dihydropyrimidin-4-yl)methyl)hexahydropyrrolo[3,4-b][1,4]oxazin-6(2H)-yl)-2,2-dimethyl-4-oxobutanoic acid (a Single Stereoisomer)

To a solution of (S)-ethyl 6-(bromomethyl)-4-(3-fluoro-2-methylphenyl)-2-(thiazol-2-yl)-1,4-dihydropyrimidine-5-carboxylate VIa-2 (100 mg, 95% purity, 0.217 mmol) in N,N-dimethylformamide (2 mL) was added 4-((4aR*,7aS*)-hexahydropyrrolo[3,4-b][1,4]oxazin-6(2H)-yl)-2,2-dimethyl-4-oxobutanoic acid I-2-2A (70 mg, 90% purity, 0.246 mmol) and N,N-diisopropylethylamine (80 mg, 0.782 mmol). After stirred at 30° C. under nitrogen atmosphere overnight, the mixture was diluted with ethyl acetate (20 mL) and washed with water (30 mL), dried over Na₂SO₄₍ₛ₎ and filtered. The filtrate was concentrated and purified by Prep. HPLC (Column: X-bridge C18 (5 um 19*150 mm); Mobile Phase A: water (+0.1% ammonium bicarbonate), Mobile Phase B: acetonitrile, UV: 214 nm, Flow rate: 15 mL/min, Gradient: 15-70% (% B)) to give a crude product, which was further purified by C18 column (acetonitrile:water (+0.1% hydrochloride)=50% to 65%) to give the title compound (55 mg, 98.7% purity, 41% yield) as yellow solids.

LC-MS (ESI): R_{T}=3.318 min, mass calcd. for C₃₀H₃₆FN₅O₆S 613.2, m/z found 614.3 [M+H]⁺. ¹H NMR (400 MHz, CD₃OD) δ 7.97 (d, J=3.2 Hz, 0.5H), 7.94 (d, J=2.8 Hz, 0.5H), 7.76 (dd, J=7.6 Hz, 2.8 Hz, 1H), 7.20-7.09 (m, 2H), 6.97-6.92 (m, 1H), 5.97 (d, J=8.4 Hz, 1H), 4.33 (br s, 0.5H), 4.29-4.19 (m, 1.5H), 4.13-3.96 (m, 4H), 3.87-3.77 (m, 2H), 3.72-3.44 (m, 4H), 3.04-2.91 (m, 1H), 2.72-2.66 (m, 1.4H), 2.64-2.55 (m, 1H), 2.55-2.51 (m, 3.6H), 1.26 (d, J=7.2 Hz, 3H), 1.21 (s, 1.4H), 1.18-1.14 (m, 4.6H).

### Compound I-3A: (S)-3-((4aR*,7aR*)-4-((5-(Ethoxycarbonyl)-6-(3-fluoro-2-methylphenyl)-2-(thiazol-2-yl)-3,6-dihydropyrimidin-4-yl)methyl)-7-oxohexahydropyrrolo[3,4-b][1,4]oxazin-6(2H)-yl)-2,2-dimethylpropanoic acid (a Single Stereoisomer)

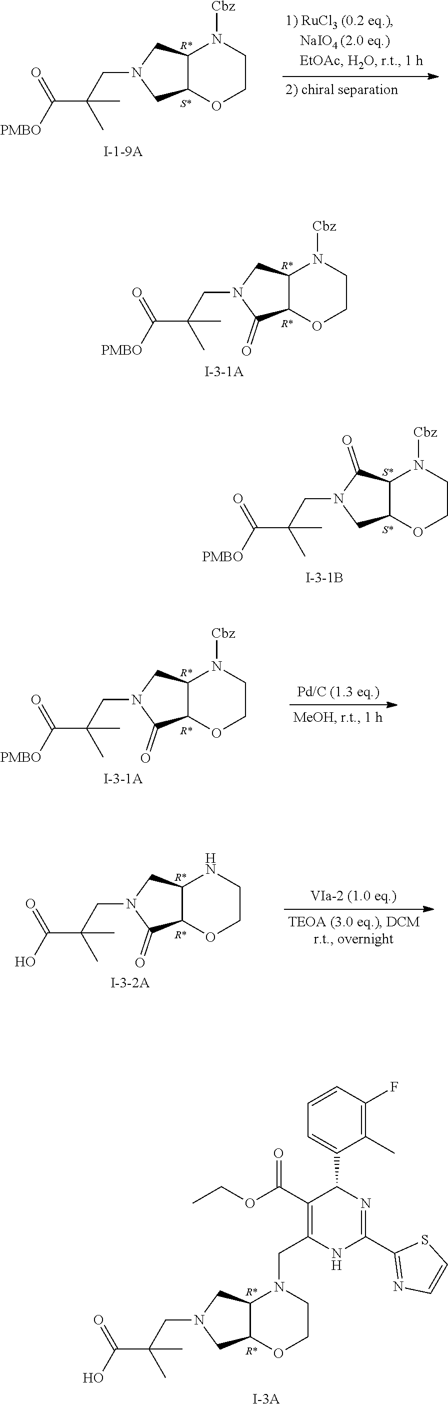

### Intermediates I-3-1A and I-3-1B: (4aR*,7aR*)-Benzyl 6-(3-((4-methoxybenzyl)oxy)-2,2-dimethyl-3-oxopropyl)-7-oxohexahydropyrrolo[3,4-b][1,4]oxazine-4(4aH)-carboxylate (a Single Stereoisomer) and (4aS*,7aS*)-benzyl 6-(3-((4-methoxybenzyl)oxy)-2,2-dimethyl-3-oxopropyl)-5-oxohexahydropyrrolo[3,4-b][1,4]oxazine-4(4aH)-carboxylate (a Single Stereoisomer)

To a solution of (4aR*,7aS*)-benzyl 6-(3-((4-methoxybenzyl)oxy)-2,2-dimethyl-3-oxopropyl)hexahydropyrrolo[3,4-b][1,4]oxazine-4 (4 aH)-carboxylate I-1-9A (500 mg, 90% purity, 0.933 mmol) in ethyl acetate (3 mL) and water (3 mL) was added sodium periodate (400 mg, 1.87 mmol) and ruthenium(III) chloride (35 mg, 0.169 mmol) at room temperature. After stirred at room temperature for 1 hour, the reaction mixture was diluted with water (10 mL) and extracted with ethyl acetate (10 mL) twice. The combined organic layers were washed with brine (10 mL), dried over Na₂SO₄₍ₛ₎, filtered and concentrated. The residue was purified by C18 column (acetonitrile:water=50% to 80%) to give a mixture compound (500 mg) as light yellow oil, which was separated by chiral Prep. HPLC (Column: Chiralpak IE 5 μm 30*250 mm; Mobile Phase: Hex:EtOH=40:60 at 30 mL/min; Temp: 35° C.; Wavelength: 230 nm) to give I-3-1A (230 mg, 90% purity from ¹H NMR, 46% yield, 100% stereopure) and I-3-1B (140 mg, 90% purity from ¹H NMR, 28% yield, 100% stereopure) as light yellow oil.

Intermediate I-3-1A: LC-MS (ESI): R_{T}=1.68 min, mass calcd. for C₂₇H₃₂N₂O₇ 496.2, m/z found 497.5 [M+H]⁺. Chiral analysis (Column: Chiralpak IE 5 um 4.6*250 mm; Mobile Phase: Hex:EtOH=40:60 at 1 mL/min; Temp: 30° C.; Wavelength: 214 nm, R_{T}=12.719 min). ¹H NMR (400 MHz, CDCl₃) δ 7.41-7.28 (m, 7H), 6.84 (d, J=8.8 Hz, 2H), 5.18 (d, J=12.4 Hz, 1H), 5.15 (d, J=12.4 Hz, 1H), 5.10-5.07 (m, 1H), 5.02-4.99 (m, 1H), 4.41-4.26 (m, 1H), 3.95-3.85 (m, 2.3H), 3.79 (s, 3H), 3.76-3.71 (m, 0.7H), 3.60 (d, J=13.6 Hz, 1H), 3.45 (td, 11.6, 2.8 Hz, 1H), 3.38 (d, J=13.6 Hz, 1H), 3.31-3.16 (m, 2H), 3.08-2.91 (m, 1H), 1.20 (s, 6H).

Intermediate I-3-1B: LC-MS (ESI): R_{T}=1.68 min, mass calcd. for C₂₇H₃₂N₂O₇ 496.2, m/z found 497.5 [M+H]⁺. Chiral analysis (Column: Chiralpak IE 5 um 4.6*250 mm; Mobile Phase: Hex:EtOH=40:60 at 1 mL/min; Temp: 30° C.; Wavelength: 214 nm, R_{T}=10.808 min). ¹H NMR (400 MHz, CDCl₃) δ 7.37-7.29 (m, 7H), 6.90-6.86 (m, 2H), 5.25-5.15 (m, 2H), 5.11-5.03 (m, 2H), 4.74 (d, J=4.4 Hz, 0.5H), 4.54 (d, J=4.4 Hz, 0.5H), 4.02-3.96 (m, 1H), 3.88-3.84 (m, 1H), 3.81 (s, 1.5H), 3.80 (s, 1.5H), 3.79-3.73 (m, 1H), 3.70-3.62 (m, 1H), 3.49-3.42 (m, 1H), 3.39-3.29 (m, 2H), 3.06-3.04 (m, 1H), 3.01-2.90 (m, 1H), 1.22-1.18 (m, 6H).

### Intermediate I-3-2A: 2,2-Dimethyl-3-((4aR*,7aR*)-7-oxohexahydropyrrolo[3,4-b][1,4]oxazin-6(2H)-yl)propanoic acid (a Single Stereoisomer)

To a solution of (4aR*,7aR*)-benzyl 6-(3-((4-methoxybenzyl)oxy)-2,2-dimethyl-3-oxopropyl)-7-oxohexahydropyrrolo[3,4-b][1,4]oxazine-4 (4aH)-carboxylate I-3-1A (200 mg, 90% purity, 0.362 mmol) in methanol (4 mL) was added 10% palladium on activated carbon (50 mg, 0.47 mmol) at room temperature. After stirred under hydrogen of balloon at room temperature for 1 hour, the reaction mixture was filtered though a pad of celite. The filtrate was concentrated to afford the title compound (80 mg, 90% purity from ¹H NMR, 82% yield) as light yellow oil.

¹H NMR (400 MHz, DMSO-d₆) δ 4.17 (d, J=4.4 Hz, 1H), 3.51-3.47 (m, 2H), 3.40-3.30 (m, 3H), 3.17-3.13 (m, 1H), 2.88 (d, J=9.2 Hz, 1H), 2.68-2.56 (m, 2H), 1.10 (s, 3H), 1.04 (s, 3H).

### Compound I-3A: (S)-3-((4aR*,7aR*)-4-((5-(Ethoxycarbonyl)-6-(3-fluoro-2-methylphenyl)-2-(thiazol-2-yl)-3,6-dihydropyrimidin-4-yl)methyl)-7-oxohexahydropyrrolo[3,4-b][1,4]oxazin-6(2H)-yl)-2,2-dimethylpropanoic acid (a Single Stereoisomer)

To a solution of (S)-ethyl 6-(bromomethyl)-4-(3-fluoro-2-methylphenyl)-2-(thiazol-2-yl)-1,4-dihydropyrimidine-5-carboxylate VIa-2 (115 mg, 95% purity, 0.249 mmol) in dichloromethane (4 mL) was added triethanolamine (112 mg, 0.751 mmol) and 2,2-dimethyl-3-((4aR*,7aR*)-7-oxohexahy dropyrrolo[3,4-b][1,4]oxazin-6(2H)-yl)propanoic acid I-3-2A (60 mg, 0.25 mmol). After stirred at 40° C. under nitrogen atmosphere overnight, the reaction mixture was cooled to room temperature, diluted with water (30 mL) and extracted with ethyl acetate (30 mL) twice. The combined organic layers were washed with brine (20 mL), dried over Na₂SO₄₍ₛ₎, filtered and concentrated to give a residue, which was purified by C18 column (acetonitrile:water=40% to 70%) to give the title compound (47 mg, 99.1% purity, 31% yield) as yellow solids.

LC-MS (ESI): R_{T}=3.323 min, mass calcd. for C₂₉H₃₄FN₅O₆S 599.2, m/z found 600.3 [M+H]⁺. ¹H NMR (400 MHz, CDCl₃) δ 9.50 (s, 1H), 7.80 (d, J=3.2 Hz, 1H), 7.41 (d, J=3.2 Hz, 1H), 7.13-7.05 (m, 2H), 6.93-6.88 (m, 1H), 6.02 (s, 1H), 4.39 (d, J=5.2 Hz, 1H), 4.33 (d, J=16.4 Hz, 1H), 4.12-4.00 (m, 2H), 3.82-3.76 (m, 3H), 3.72-3.62 (m, 2H), 3.40-3.36 (m, 1H), 3.27 (d, J=14.0 Hz, 1H), 3.21-3.19 (m, 1H), 2.59-2.54 (m, 1H), 1.51 (s, 3H), 2.48-2.42 (m, 1H), 1.31 (s, 3H), 1.28 (s, 3H), 1.12 (t, J=7.2 Hz, 3H).

### Compound I-3B: (S)-3-((4aS*,7aS*)-4-((5-(Ethoxycarbonyl)-6-(3-fluoro-2-methylphenyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

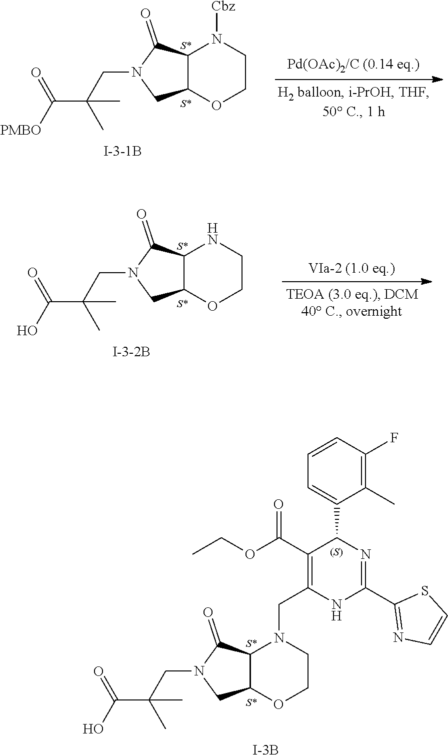

### Intermediate I-3-2B: 2,2-Dimethyl-3-((4aS*,7aS*)-5-oxohexahydropyrrolo[3,4-b][1,4]oxazin-6(2H)-yl)propanoic acid (a Single Stereoisomer)

To a solution of (4aS*,7aS*)-benzyl 6-(3-((4-methoxybenzyl)oxy)-2,2-dimethyl-3-oxopropyl)-5-oxohexahydropyrrolo[3,4-b][1,4]oxazine-4(4aH)-carboxylate I-3-1B (140 mg, 90% purity, 0.254 mmol) in 2-propanol (2 mL) and tetrahydrofuran (2 mL) was added palladium hydroxide (50 mg, 0.036 mmol) at room temperature. After stirred under hydrogen atmosphere (balloon) at 50° C. for 1 hour, the reaction mixture was filtered and concentrated to afford the title compound (60 mg, 30% purity from ¹H NMR, 29% yield) as light yellow oil.

¹H NMR (400 MHz, CDCl₃) δ 4.14-4.12 (m, 1H), 3.67-3.49 (m, 4H), 3.30-3.18 (m, 2H), 2.78-2.72 (m, 1H), 2.68-2.65 (m, 1H), 2.52-2.48 (m, 1H), 1.23 (s, 3H), 1.19 (s, 3H).

### Compound I-3B: (S)-3-((4aS*,7aS*)-4-((5-(Ethoxycarbonyl)-6-(3-fluoro-2-methylphenyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

To the solution of (S)-ethyl 6-(bromomethyl)-4-(3-fluoro-2-methylphenyl)-2-(thiazol-2-yl)-1,4-dihydropyrimidine-5-carboxylate VIa-2 (37 mg, 90% purity, 0.076 mmol) in dichloromethane (4 mL) was added triethanolamine (34 mg, 0.228 mmol) and 2,2-dimethyl-3-((4aS*,7aS*)-5-oxohexahydropyrrolo[3,4-b][1,4]oxazin-6(21-1)-yl)propanoic acid I-3-2B (60 mg, 30% purity, 0.074 mmol). After stirred at 40° C. under nitrogen atmosphere overnight, the reaction mixture was cooled to room temperature, diluted with water (30 mL) and extracted with ethyl acetate (30 mL) twice. The combined organic layers were washed with brine (20 mL), dried over Na₂SO₄₍ₛ₎, filtered and concentrated to give a residue, which was purified by C18 column (acetonitrile:water=40% to 70%) to give the title compound (18.4 mg, 95.9% purity, 39% yield) as yellow solids. LC-MS (ESI): R_{T}=3.509 min, mass calcd. for C₂₉H₃₄FN₅O₆S 599.2, m/z found 600.3 [M+H]⁺. ¹H NMR (400 MHz, CDCl₃) δ 9.48 (s, 1H), 7.83 (d, J=3.2 Hz, 1H), 7.41 (d, J=3.2 Hz, 1H), 7.10-7.03 (m, 1H), 6.99-6.97 (m, 1H), 6.91-6.87 (m, 1H), 6.01 (s, 1H), 4.65 (d, J=17.6 Hz, 1H), 4.54 (d, J=17.6 Hz, 1H), 4.29-4.26 (m, 1H), 4.11-4.03 (m, 2H), 3.89-3.76 (m, 2H), 3.63-3.55 (m, 2H), 3.44 (d, J=3.6 Hz, 1H), 3.39-3.36 (m, 2H), 2.96-2.90 (m, 1H), 2.57 (s, 1H), 2.54 (s, 3H), 1.26 (s, 3H), 1.23 (s, 3H), 1.15 (t, J=7.2 Hz, 3H).

### Compound I-4E: (S)-ethyl 6-(((cis)-6-acetylhexahydropyrrolo[3,4-b][1,4]oxazin-4(4aH)-yl)methyl)-4-(3-fluoro-2-methylphenyl)-2-(thiazol-2-yl)-1,4-dihydropyrimidine-5-carboxylate (a Mixture of 2 Stereoisomers)

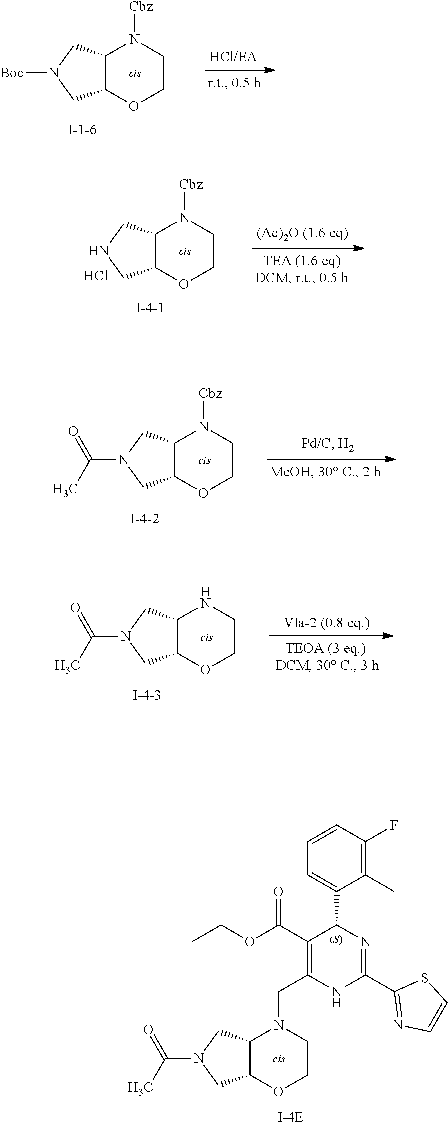

### Intermediate I-4-1: (cis)-benzyl hexahydropyrrolo[3,4-b][1,4]oxazine-4(4aH)-carboxylate hydrochloride (a Mixture of 2 Stereoisomers)

The solution of (cis)-4-benzyl 6-tert-butyl hexahydropyrrolo[3,4-b][1,4]oxazine-4,6-dicarboxylate I-1-6 (250 mg, 90% purity, 0.621 mmol) in 4 M hydrochloride in ethyl acetate (2 mL) was stirred at room temperature for 0.5 hour. the mixture was concentrated under reduced pressure to give the title compound (180 mg, 97% yield) as purple solids which was directly used in next step without further purification.

LC-MS (ESI): R_{T}=1.31 min, mass calcd. for C₁₄H₁₈N₂O₃ 262.1, m/z found 263.0 [M+H]⁺.

### Intermediate I-4-2: (cis)-benzyl 6-acetylhexahydropyrrolo[3,4-b][1,4]oxazine-4(4aH)-carboxylate (a Mixture of 2 Stereoisomers)

To a suspension of (cis)-benzyl hexahydropyrrolo[3,4-b][1,4]oxazine-4(4aH)-carboxylate hydrochloride I-4-1 (180 mg, 0.602 mmol) in dichloromethane (2 mL) was added triethylamine (95 mg, 0.941 mmol). The mixture was stirred at room temperature for 15 minutes before acetic anhydride (94 mg, 0.922 mmol) was added. After stirred at room temperature for 1 hour, the mixture was poured into water (10 ml) and extracted with dichloromethane (5 mL) twice. The combined organic layers were washed with brine (10 mL), dried over Na₂SO₄₍ₛ₎ and filtered. The filtrate was concentrated and purified by silical gel column chromatography (dichloromethane:methonal=20:1) to get the title compound (165 mg, 90% purity from ¹H NMR, 81% yield) as purple oil.

¹H NMR (400 MHz, CDCl₃) δ 7.37-7.33 (m, 5H), 5.22-5.09 (m, 2H), 4.62-4.41 (m, 1H), 4.04-3.47 (m, 8H), 3.25-3.07 (m, 1H), 2.05-2.02 (m, 3H).

### Intermediate I-4-3: 1-((cis)-hexahydropyrrolo[3,4-b][1,4]oxazin-6(2H)-yl)ethenone (a Mixture of 2 Stereoisomers)

To a solution of (cis)-benzyl 6-acetylhexahydropyrrolo[3,4-b][1,4]oxazine-4(4aH)-carboxylate I-4-2 (165 mg, 90% purity, 0.488 mmol) in methanol (5 mL) was added 10% palladium on charcoal wt. (50 mg). After stirred at 30° C. under hydrogen atmosphere for 2 hours, the mixture was filtered. The filtrate was concentrated to give the title compound (80 mg, 90% purity from ¹H NMR, 87% yield) as colorless oil which was directly used in next step without further purification.

¹H NMR (400 MHz, CDCl₃) δ 4.09-4.07 (m, 0.5H), 3.98 (t, J=4.0 Hz, 0.5H), 3.86 (t, J=2.8 Hz, 0.5H), 3.83 (t, J=2.8 Hz, 0.5H), 3.75 (t, J=9.6 Hz, 0.5H), 3.67-3.52 (m, 4.5H), 3.48-3.43 (m, 1H), 3.16-3.09 (m, 1H), 2.73-2.68 (m, 1H), 2.08-2.06 (m, 3H).

### Compound I-4E: (S)-ethyl 6-(((cis)-6-acetylhexahydropyrrolo[3,4-b][1,4]oxazin-4(4aH)-yl)methyl)-4-(3-fluoro-2-methylphenyl)-2-(thiazol-2-yl)-1,4-dihydropyrimidine-5-carboxylate (a Mixture of 2 Stereoisomers)

To a solution of 1-((cis)-hexahydropyrrolo[3,4-b][1,4]oxazin-6(2H)-yl)ethanone I-4-3 (50 mg, 90% purity, 0.264 mmol) and (S)-ethyl 6-(bromomethyl)-4-(3-fluoro-2-methylphenyl)-2-(thiazol-2-yl)-1,4-dihydropyrimidine-5-carboxylate VIa-2 (93 mg, 95% purity, 0.202 mmol) in dichloromethane (2 mL) was added triethanolamine (119 mg, 0.798 mmol) at room temperature. After stirred at 30° C. under nitrogen atmosphere for 3 hours, the mixture was concentrated and purified by C18 column (acetonitrile:water=25% to 70%) to give the title compound (60 mg, 99.7% purity, 56% yield) as yellow solids.

LC-MS (ESI): R_{T}=3.851 min, mass calcd. for C₂₆H₃₀FN₅O₄S 527.2, m/z found 528.3 [M+H]⁺. ¹H NMR (400 MHz, CDCl₃) δ 9.52-9.37 (m, 1H), 7.87-7.77 (m, 1H), 7.46-7.40 (m, 1H), 7.11-7.02 (m, 1H), 6.96-6.94 (m, 1H), 6.90-6.86 (m, 1H), 6.06-5.95 (m, 1H), 4.38-4.14 (m, 2H), 4.11-3.87 (m, 4H), 3.86-3.39 (m, 6H), 3.03-2.81 (m, 1H), 2.85-2.58 (m, 0.5H), 2.55 (s, 3H), 2.50-2.38 (m, 0.5H), 2.10-1.99 (m, 3H), 1.17-1.07 (m, 3H).

### Compound 1-5: 3-((cis)-4-4(S)-5-(ethoxycarbonyl)-6-(3-fluoro-2-methylphenyl)-2-(thiazol-2-yl)-3,6-dihydropyrimidin-4-yl)methyphexahydropyrrolo[3,4-b][1,4]oxazin-6(2H)-yl)cyclopentanecarboxylic acid (a Mixture of 8 Stereoisomers)

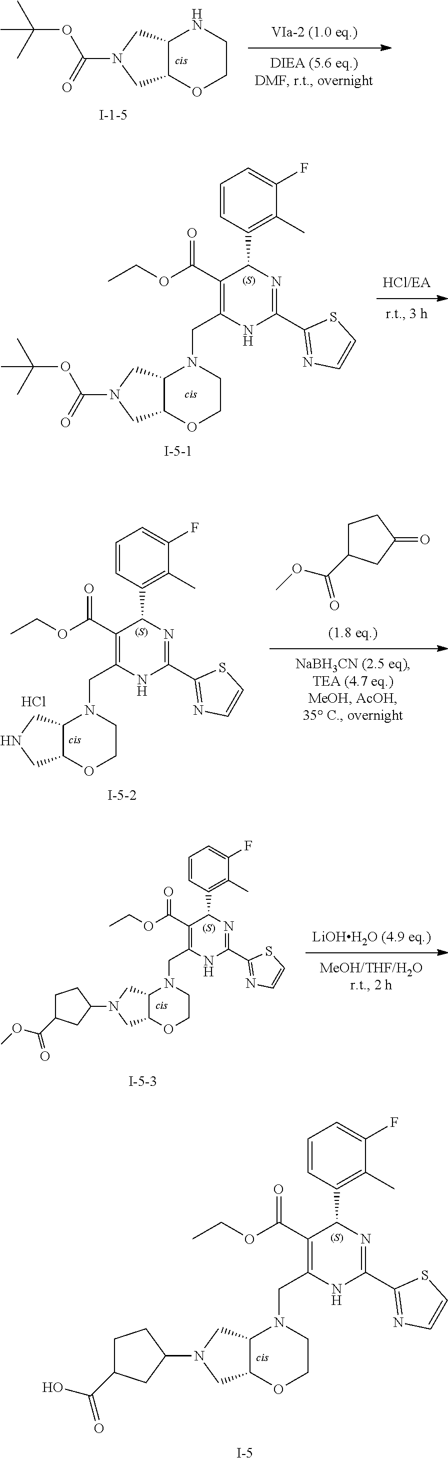

### Intermediate I-5-1: (cis)-tert-butyl 4-4(S)-5-(ethoxycarbonyl)-6-(3-fluoro-2-methylphenyl)-2-(thiazol-2-yl)-3,6-dihydropyrimidin-4-yl)methyphexahydropyrrolo[3,4-b][1,4]oxazine-6(2H)-carboxylate (a Mixture of 2 Stereoisomers)

To a solution of (S)-ethyl 6-(bromomethyl)-4-(3-fluoro-2-methylphenyl)-2-(thiazol-2-yl)-1,4-dihydropyrimidine-5-carboxylate VIa-2 (100 mg, 95% purity, 0.217 mmol) in N,N-dimethylformamide (3 mL) was added (cis)-tert-butyl hexahydropyrrolo[3,4-b][1,4]oxazine-6(2H)-carboxylate I-1-5 (72 mg, 0.284 mmol) and N,N-diisopropylethylamine (0.2 mL, 1.21 mmol) at room temperature. After stirred at room temperature overnight, the mixture was purified by C18 column (acetonitrile:water=10% to 100%) to give the title compound (130 mg, 90% purity from ¹H NMR, 92% yield) as yellow solids.

¹H NMR (400 MHz, CDCl₃) δ 9.57-9.51 (m, 1H), 7.83-7.82 (m, 1H), 7.43 (s, 1H), 7.08-6.89 (m, 3H), 6.03-6.02 (m, 1H), 4.37-3.79 (m, 7H), 3.65-3.29 (m, 5H), 3.02-2.82 (m, 1H), 2.60-2.55 (m, 3.3H), 2.43-2.40 (m, 0.7H), 1.46-1.43 (m, 9H), 1.14-1.13 (m, 3H).

### Intermediate I-5-2: (S)-ethyl 4-(3-fluoro-2-methylphenyl)-6-(((cis)-hexahydropyrrolo[3,4-b][1,4]oxazin-4(4aH)-yl)methyl)-2-(thiazol-2-yl)-1,4-dihydropyrimidine-5-carboxylate hydrochloride (a Mixture of 2 Stereoisomers)

A solution of (cis)-tert-butyl 4-4(S)-5-(ethoxycarbonyl)-6-(3-fluoro-2-methylphenyl)-2-(thiazol-2-yl)-3,6-dihydropyrimidin-4-yl)methyphexahydropyrrolo[3,4-b][1,4]oxazine-6(2H)-carboxylate I-5-1 (130 mg, 90% purity, 0.2 mmol) in 4 M hydrochloride in ethyl acetate (10 mL) was stirred at room temperature for 3 hours. The mixture was concentrated to give the title compound (110 mg, 85% purity, 84% yield) as yellow solids.

LC-MS (ESI): R_{T}=1.598 min, mass calcd. for C₂₄H₂₈FN₅O₃S 485.2, m/z found 486.2 [M+H]⁺.

### Intermediate I-5-3: (4S)-ethyl 4-(3-fluoro-2-methylphenyl)-6-(((cis)-6-(3-(methoxycarbonyl)cyclopentyl) hexahydropyrrolo[3,4-b][1,4]oxazin-4(4aH)-yl)methyl)-2-(thiazol-2-yl)-1,4-dihydropyrimidine-5-carboxylate (a Mixture of 8 Stereoisomers)

To a solution of (S)-ethyl 4-(3-fluoro-2-methylphenyl)-6-(((cis)-hexahydropyrrolo[3,4-b][1,4]oxazin-4 (4aH)-yl)methyl)-2-(thiazol-2-yl)-1,4-dihydropyrimidine-5-carboxylate hydrochloride I-5-2 (50 mg, 85% purity, 0.076 mmol) in methanol (5 mL) was added triethylamine (0.05 mL, 0.359 mmol) at room temperature. The mixture was stirred at room temperature for 30 minutes before methyl 3-oxocyclopentanecarboxylate (21 mg, 95% purity, 0.14 mmol) was added. After stirred at 60° C. for 2 hours, the mixture was cooled down to room temperature, and then acetic acid (0.2 mL) and sodium cyanoborohydride (12 mg, 0.191 mmol) was added. After stirred at 35° C. under nitrogen atmosphere overnight, the resulting mixture was quenched with water (30 mL) and extracted with ethyl acetate (20 mL) for three times. The combined organic layers were washed with brine (30 mL), dried over Na₂SO₄₍ₛ₎ and filtered. The filtrate was concentrated and purified by C18 column (acetonitrile:water=10% to 90%) to give the title compound (30 mg, 90% purity from ¹H NMR, 58% yield) as yellow solids.

LC-MS (ESI): R_{T}=1.908 min, mass calcd. for C₃₁H₃₈FN₅O₅S 611.3, m/z found 612.3 [M+H]⁺. ¹H NMR (400 MHz, CD₃OD) δ 8.36-8.23 (m, 2H), 7.38-7.31 (m, 2H), 7.14-7.09 (m, 1H), 6.25-6.18 (m, 1H), 4.40-4.26 (m, 2H), 4.14-4.12 (m, 3H), 4.06-3.99 (m, 2H), 3.93-3.79 (m, 3H), 3.71-3.69 (m, 3H), 3.63-3.56 (m, 1H), 3.24-3.19 (m, 3H), 3.14-3.08 (m, 1H), 3.04-2.94 (m, 1H), 2.88-2.75 (m, 1H), 2.50 (s, 3H), 2.29-2.17 (m, 1.3H), 2.11-2.00 (m, 2.7H), 1.92-1.80 (m, 1H), 1.17-1.07 (m, 3H).

### Compound I-5: 3-((cis)-4-4(S)-5-(ethoxycarbonyl)-6-(3-fluoro-2-methylphenyl)-2-(thiazol-2-yl)-3,6-dihydropyrimidin-4-yl)methyphexahydropyrrolo[3,4-b][1,4]oxazin-6(2H)-yl)cyclopentanecarboxylic acid (a Mixture of 8 Stereoisomers)

To a solution of (4S)-ethyl 4-(3-fluoro-2-methylphenyl)-6-(((cis)-6-(3-(methoxycarbonyl) cyclopentyl)hexahydropyrrolo[3,4-b][1,4]oxazin-4(4aH)-yl)methyl)-2-(thiazol-2-yl)-1,4-dihydropyrimidine-5-carboxylate I-5-3 (30 mg, 90% purity, 0.044 mmol) in methanol (3 mL) and tetrahydrofuran (3 mL) was added a solution of lithium hydroxide monohydrate (9 mg, 0.214 mmol) in water (1 mL) at room temperature. After stirred at room temperature for 2 hours, the reaction mixture was diluted with water (5 mL) and acidified with 1M hydrochloride aqueous solution to pH=2. Then the mixture was extracted with ethyl acetate (15 mL) for three times. The combined organic layers were washed with brine (20 mL), dried over Na₂SO₄₍ₛ₎ and filtered. The filtrate was concentrated and purified by C18 column (acetonitrile:water=10% to 90%) to give the title compound (12 mg, 97.1% purity, 44% yield) as yellow solids.

LC-MS (ESI): R_{T}=3.342 min, 3.474 min, mass calcd. for C₃₀H₃₆FN₅O₅S 597.2, m/z found 598.3 [M+H]⁺. ¹H NMR (400 MHz, CD₃OD) δ 7.93 (d, J=3.2 Hz, 1H), 7.73 (d, J=3.2 Hz, 1H), 7.17-7.07 (m, 2H), 6.96-6.91 (m, 1H), 5.99 (s, 0.5H), 5.97 (s, 0.5H), 4.35-4.21 (m, 2H), 4.09-3.99 (m, 3.3H), 3.94-3.89 (m, 0.7H), 3.82-3.77 (m, 2H), 3.70-3.63 (m, 1H), 3.58-3.35 (m, 3H), 3.25-3.14 (m, 1H), 3.05-2.79 (m, 2H), 2.68-2.65 (m, 0.5H), 2.51-2.44 (m, 3.5H), 2.34-2.24 (m, 0.4H), 2.17-1.95 (m, 4.6H), 1.87-1.75 (m, 0.7H), 1.64-1.55 (m, 0.3H), 1.13 (t, J=7.2 Hz, 3H).

### Compound I-6A: 3-((4aR*,7aS*)-4-4(S)-5-(Ethoxycarbonyl)-6-(3-fluoro-2-methylphenyl)-2-(thiazol-2-yl)-3,6-dihydropyrimidin-4-yl)methyphexahydropyrrolo[3,4-b][1,4]oxazin-6(2H)-yl)-2,2-dimethylpropanoic acid (a Single Stereoisomer)

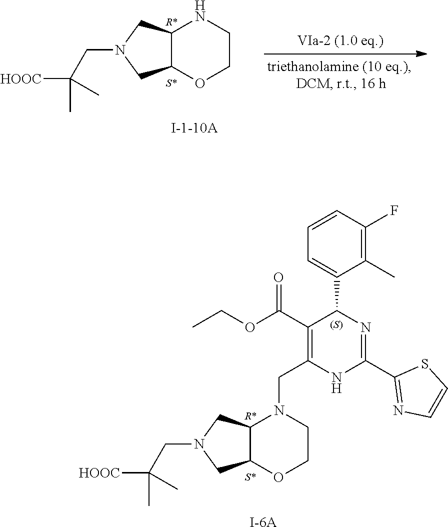

To a solution of 3-((4aR*,7aS*)-hexahydropyrrolo[3,4-b][1,4]oxazin-6(2H)-yl)-2,2-dimethylpropanoic acid I-1-10A (105 mg, 80% purity, 0.317 mmol) and triethanolamine (485 mg, 3.25 mmol) in dichloromethane (10 mL) was added (S)-ethyl 6-(bromomethyl)-4-(3-fluoro-2-methylphenyl)-2-(thiazol-2-yl)-1,4-dihydropyrimidine-5-carboxylate VIa-2 (150 mg, 95% purity, 0.325 mmol) at room temperature. After stirred at room temperature for 16 hours, the mixture was concentrated under reduced pressure to give a residue, which was purified by C18 column (acetonitrile:water (+0.1% ammonium bicarbonate)=05% to 80%) to give the title compound (116.1 mg, 98.6% purity, 60% yield) as yellow solids.

LC-MS (ESI): R_{T}=3.745 min, mass calcd. for C₂₉H₃₆FN₅O₅S 585.2, m/z found 586.3 [M+H]⁺. ¹H NMR (400 MHz, CD₃OD) δ 7.92 (d, J=3.2 Hz, 1H), 7.73 (d, J=3.2 Hz, 1H), 7.16-7.07 (m, 2H), 6.95-6.91 (m, 1H), 5.96 (s, 1H), 4.30 (d, J=16.4 Hz, 1H), 4.25 (d, J=2.0 Hz, 1H), 4.08-3.98 (m, 4H), 3.89-3.79 (m, 2H), 3.77-3.71 (m, 1H), 3.44 (s, 2H), 3.39-3.32 (m, 1H), 3.24 (d, J=13.2 Hz, 1H), 3.14 (d, J=13.2 Hz, 1H), 3.03 (td, J=12.0, 2.8 Hz, 1H), 2.68 (d, J=11.6 Hz, 1H), 2.51 (s, 3H), 1.28 (s, 3H), 1.20 (s, 3H), 1.12 (t, J=7.2 Hz, 3H).

### Compound I-7F: (S)-(cis)-Ethyl 4-(3-fluoro-2-methylphenyl)-6-((6-(methylsulfonyl)hexahydropyrrolo[3,4-b][1,4]oxazin-4(4aH)-yl)methyl)-2-(thiazol-2-yl)-1,4-dihydropyrimidine-5-carboxylate (a Mixture of 2 Stereoisomers)

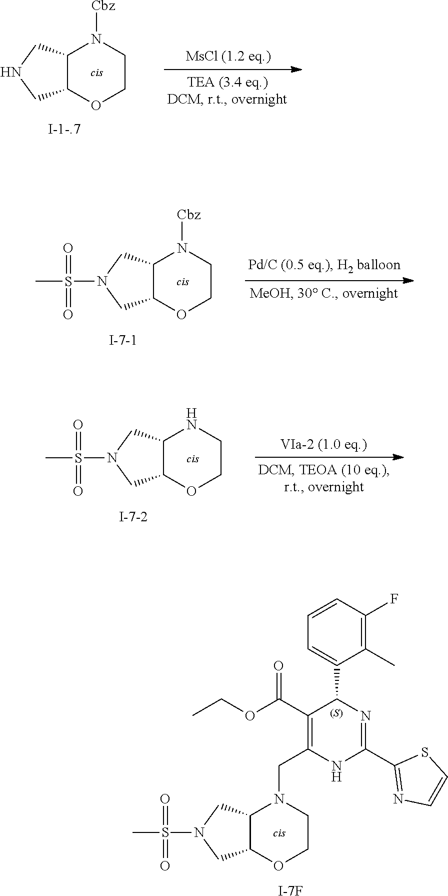

### Intermediate I-7-1: (cis)-Benzyl 6-(methylsulfonyl)hexahydropyrrolo[3,4-b][1,4]oxazine-4(4aH)-carboxylate (a Mixture of 2 Stereoisomers)

To a solution of (cis)-benzyl hexahydropyrrolo[3,4-b][1,4]oxazine-4(4aH)-carboxylate I-1-7 (140 mg, 90% purity, 0.480 mmol) in dichloromethane (3 mL) was added methanesulfonyl chloride (65 mg, 0.567 mmol) and triethylamine (166 mg, 1.64 mmol) at room temperature. After stirred at room temperature overnight, the mixture was dissolved in dichloromethane (30 mL) and washed with brine (30 mL), dried over Na₂SO₄(s) and filtered. The filtrate was concentrated under reduced pressure to give a residue, which was purified by C18 column (acetonenitrile:water=05% to 95%) to give the title compound (130 mg, 68% yield, 85% purity from LCMS) as white oil. LC-MS (ESI): R_{T}=1.47 min, mass calcd. for C₁₅H₂₀N₂O₅S 340.1, found 341.0 [M+H]⁺.

### Intermediate I-7-2: (cis)-6-(Methylsulfonyl)octahydropyrrolo[3,4-b][1,4]oxazine (a Mixture of 2 Stereoisomers)

To s solution of (cis)-benzyl 6-(methylsulfonyl)hexahydropyrrolo[3,4-b][1,4]oxazine-4(4aH)-carboxylate I-7-1 (130 mg, 85% purity, 0.325 mmol) in methanol (15 mL) was added 10% wt. palladium on activated carbon (17 mg, 0.16 mmol). After stirred at 30° C. under hydrogen atmosphere (balloon) overnight, the mixture was filtered through a pad of celite. The filtrate was concentrated under reduced pressure to give the title compound (65 mg, 95% purity from ¹H NMR, 92% yield) as colorless oil.

¹H NMR (400 MHz, CDCl₃) δ 3.98 (t, J=3.2 Hz, 1H), 3.86-3.82 (m, 1H), 3.60-3.49 (m, 4H), 3.42-3.37 (m, 2H), 3.13-3.07 (m, 1H), 2.87 (s, 3H), 2.71-2.67 (m, 1H).

### Compound I-7F: (S)-(cis)-Ethyl 4-(3-fluoro-2-methylphenyl)-6-((6-(methylsulfonyl)hexahydropyrrolo[3,4-b][1,4]oxazin-4 (4 aH)-yl)methyl)-2-(thiazol-2-yl)-1,4-dihydropyrimidine-5-carboxylate (a Mixture of 2 Stereoisomers)

Converted from compounds I-7-2 and VIa-2. By utilizing the analogous procedure of Method C, the title compound was synthesized as yellow solid.

LC-MS (ESI): R_{T}=3.914 min, mass calcd. for C₂₅H₃₀FN₅O₅S₂ 564.2, found 564.2 [M+H]⁺. ¹H NMR (400 MHz, CD₃OD) δ 7.94 (d, J=2.8 Hz, 1H), 7.74 (d, J=3.2 Hz, 1H), 7.16-7.09 (m, 2H), 6.95 (t, J=8.4 Hz, 1H), 6.00 (s, 0.5H), 5.98 (s, 0.5H), 4.31-4.19 (m, 2H), 4.11-4.05 (m, 3H), 4.00-3.90 (m, 1.5H), 3.81 (t, J=11.2 Hz, 1H), 3.71-3.65 (m, 1.5H), 3.58-3.49 (m, 2H), 3.45-3.41 (m, 2H), 3.02 (td, J=11.6, 3.2 Hz, 0.5H), 2.92 (s, 1.5H), 2.91 (s, 1.5H), 2.66 (d, J=12.0 Hz, 0.5H), 2.53 (s, 3H), 1.15 (t, J=5.2 Hz, 3H).

### Compound I-8F: 2-((cis)-4-(((S)-5-(Ethoxycarbonyl)-6-(3-fluoro-2-methylphenyl)-2-(thiazol-2-yl)-3,6-dihydropyrimidin-4-yl)methyl)hexahydropyrrolo[3,4-b][1,4]oxazin-6(2H)-yl)thiazole-5-carboxylic acid (a Mixture of 2 Stereoisomers)

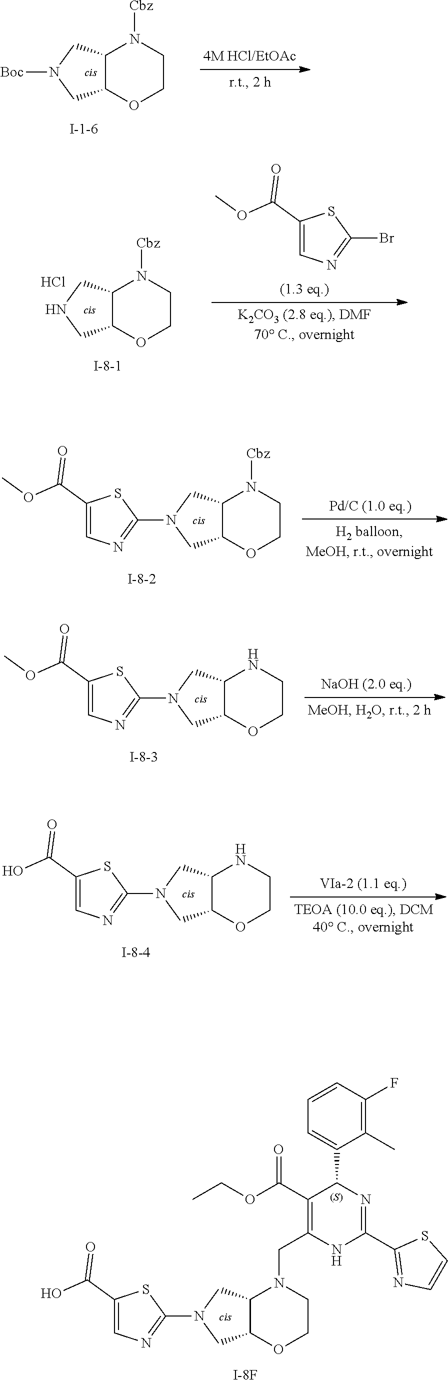

### Intermediate I-8-1: (cis)-Benzyl hexahydropyrrolo[3,4-b][1,4]oxazine-4(4aH)-carboxylate hydrochloride (a Mixture of 2 Stereoisomers)

To a solution of (cis)-4-benzyl 6-tert-butyl hexahydropyrrolo[3,4-b][1,4]oxazine-4,6-dicarboxylate I-1-6 (460 mg, 90% purity, 1.14 mmol) in ethyl acetate (2 mL) was added 4.0 M hydrochloride in ethyl acetate (5 mL) under nitrogen atmosphere. After stirred at room temperature for 2 hours, the mixture was concentrated to give the title compound (380 mg, 90% purity from ¹H NMR, 99% yield) as white solids.

LC-MS (ESI): R_{T}=1.108 min, mass calcd. for C₁₄H₁₉ClN₂O₃ 298.1, m/z found 263.1 [M−HCl+H]⁺. ¹H NMR (400 MHz, DMSO-d₆) δ 9.45 (s, 1H), 9.18 (s, 1H), 7.38-7.33 (m, 5H), 5.14 (s, 2H), 4.61-4.55 (m, 1H), 4.07 (s, 1H), 3.92-3.86 (m, 1H), 3.70-3.67 (m, 1H), 3.53-3.50 (m, 1H), 3.47-3.42 (m, 1H), 3.39-3.32 (m, 2H), 3.23-3.19 (m, 2H).

### Intermediate I-8-2: (cis)-Benzyl 6-(5-(methoxycarbonyl)thiazol-2-yl)hexahydropyrrolo[3,4-b][1,4]oxazine-4(4aH)-carboxylate (a Mixture of 2 Stereoisomers)

To a solution of (cis)-benzyl hexahydropyrrolo[3,4-b][1,4]oxazine-4(4aH)-carboxylate hydrochloride I-8-1(380 mg, 90% purity, 1.14 mmol) in N,N-dimethylformamide (3 mL) was added potassium carbonate (438 mg, 3.18 mmol), 2-bromo-5-(methoxymethyl)thiazole (315 mg, 1.52 mmol) at room temperature. After stirred at 70° C. overnight under nitrogen atmosphere, the mixture was diluted with water (20 mL) and extracted with dichloromethane (20 mL) for three times. The combined organic layers were washed with brine (20 mL), dried over Na₂SO₄₍ₛ₎ and filtered. The filtrate was concentrated under reduced pressure to give a residue, which was purified by silica gel column chromatography (petroleum ether:ethyl acetate=10:1 to 5:1) to give the title compound (360 mg, 90% purity from ¹H NMR, 70% yield) as yellow oil.

LC-MS (ESI): R_{T}=1.127 min, mass calcd. for C₁₉H₂₁N₃O₅S 403.1, m/z found 404.1 [M+H]⁺. ¹H NMR (400 MHz, CDCl₃) δ 7.89 (s, 1H), 7.40-7.35 (m, 5H), 5.18-5.17 (m, 2H), 4.75-4.63 (m, 1H), 4.12 (s, 1H), 3.99-3.89 (m, 2H), 3.83 (s, 3H), 3.76-3.69 (m, 3H), 3.59-3.53 (m, 2H), 3.26-3.10 (m, 1H).

### Intermediate I-8-3: (cis)-Methyl 2-hexahydropyrrolo[3,4-b][1,4]oxazin-6(2H)-yl)thiazole-5-carboxylate (a Mixture of 2 Stereoisomers)

To a solution of (cis)-Benzyl 6-(5-(methoxycarbonyl)thiazol-2-yl)hexahydropyrrolo[3,4-b][1,4]oxazine-4(4aH)-carboxylate I-8-2 (120 mg, 90% purity, 0.267 mmol) in methanol (4 mL) was added 10% wt. palladium on charcoal (285 mg, 0.267 mmol) at room temperature. After stirred at room temperature under hydrogen atmosphere balloon overnight, the mixture was filtered through a pad of celite. The filtrate was concentrated under reduced pressure to give the title compound (70 mg, 90% purity from ¹H NMR, 88% yield) as yellow oil. ¹H NMR (400 MHz, CDCl₃) δ 7.90 (s, 1H), 4.15-4.13 (m, 1H), 3.88-3.85 (m, 1H), 3.83 (s, 3H), 3.79-3.75 (m, 1H), 3.70-3.64 (m, 3H), 3.60-3.57 (m, 1H), 3.53-3.48 (m, 1H), 3.19-3.11 (m, 1H), 2.74-2.71 (m, 1H).

### Intermediate I-8-4: (cis)-2-Hexahydropyrrolo[3,4-b][1,4]oxazin-6(2H)-yl)thiazole-5-carboxylic acid (a Mixture of 2 Stereoisomers)

To a solution of (cis)-methyl 2-hexahydropyrrolo[3,4-b][1,4]oxazin-6(2H)-yl)thiazole-5-carboxylate I-8-3 (70 mg, 90% purity, 0.23 mmol) in methanol (1 mL) was added sodium hydroxide (19 mg, 0.48 mmol) in water (1 mL) at room temperature under nitrogen atmosphere. After stirred at room temperature for 2 hours, the mixture was diluted with water (10 mL) and extracted with ethyl acetate (10 mL) for three times. The combined organic layers were washed with brine (20 mL), dried over Na₂SO₄₍ₛ₎ and filtered. The filtrate was concentrated to give the title compound (50 mg, 90% purity from ¹H NMR, 75% yield) as yellow solids.

LC-MS (ESI): R_{T}=0.258 min, mass calcd. for CioHi3N303S 255.1, m/z found 256.1 [M+H]⁺. ¹H NMR (400 MHz, DMSO-d₆) δ 10.77 (s, 1H), 10.29 (s, 1H), 7.81 (s, 1H), 4.45-4.43 (m, 1H), 4.09-4.08 (m, 1H), 3.94-3.79 (m, 4H), 3.70-3.66 (m, 1H), 3.50-3.47 (m, 1H), 3.30-3.25 (m, 1H), 3.07-3.04 (m, 1H).

### Compound I-8F: 2-((cis)-4-4(S)-5-(Ethoxycarbonyl)-6-(3-fluoro-2-methylphenyl)-2-(thiazol-2-yl)-3,6-dihydropyrimidin-4-yl)methyphexahydropyrrolo[3,4-b][1,4]oxazin-6(2H)-yl)thiazole-5-carboxylic acid (a Mixture of 2 Stereoisomers)

Converted from compounds I-8-4 and VIa-2. By utilizing the analogous procedure of Method C, the title compound was synthesized as yellow solid.

LC-MS (ESI): R_{T}=3.597 min, mass calcd. for C₂₈H₂₉FN₆O₅S₂ 612.2, m/z found 613.1 [M+H]⁺. ¹H NMR (400 MHz, CD₃OD) δ 7.93-7.91 (m, 1H), 7.72-7.70 (m, 2H), 7.18-7.09 (m, 2H), 6.95-6.88 (m, 1H), 5.98 (s, 0.6H), 5.96 (s, 0.4H), 4.42-4.36 (m, 1.6H), 4.35-4.29 (m, 0.4H), 4.25-4.07 (m, 3H), 4.02-3.95 (m, 1H), 3.94-3.81 (m, 2H), 3.79-3.75 (m, 1H), 3.74-3.65 (m, 2H), 3.60-3.55 (m, 1H), 3.01-2.90 (m, 1H), 2.70 (d, J=10.0 Hz, 0.4H), 2.57 (d, J=12.0 Hz, 0.6H), 2.50 (s, 3H), 1.15 (t, J=7.2 Hz, 3H).

### Compound I-9F: (4aS*,7aR*)—(S)-3-((4-((5-(Ethoxycarbonyl)-6-(3-fluoro-2-methylphenyl)-2-(thiazol-2-yl)-3,6-dihydropyrimidin-4-yl)methyphexahydropyrrolo[3,4-b][1,4]oxazin-6(2H)-yl)sulfonyl)-1-methylcyclobutanecarboxylic acid (a Mixture of 2 Stereoisomers)

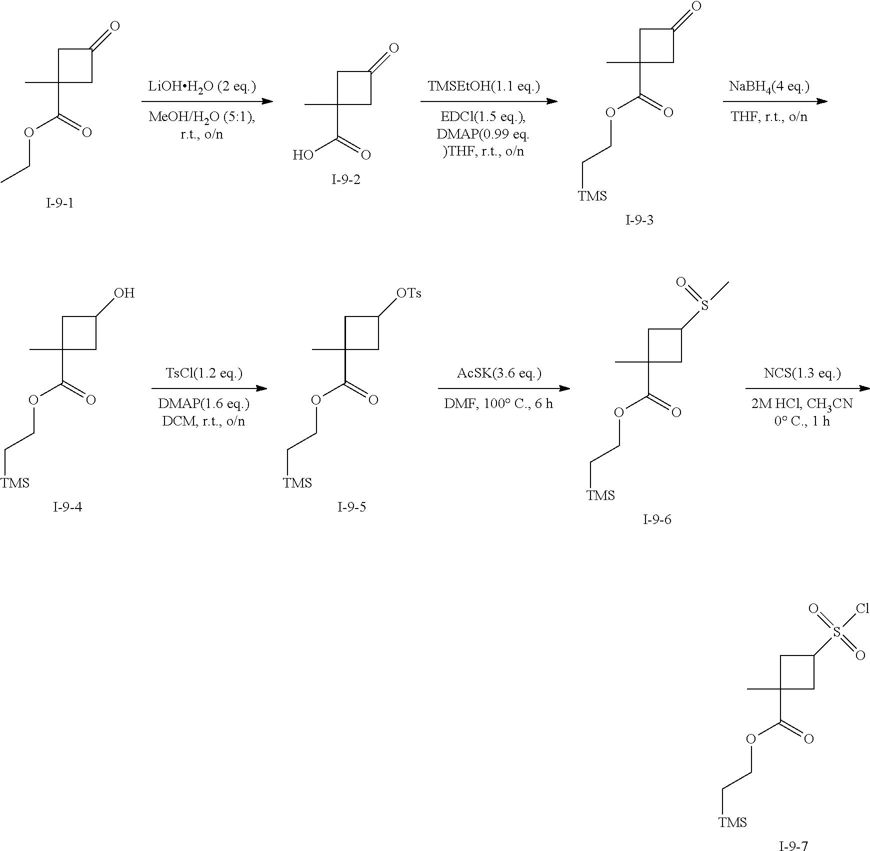

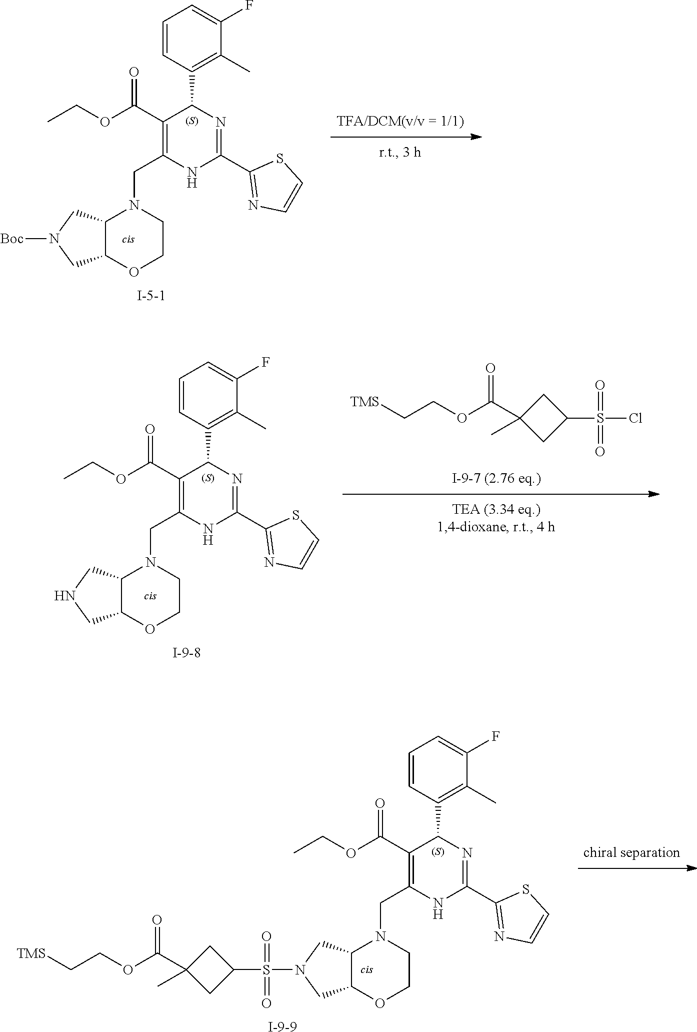

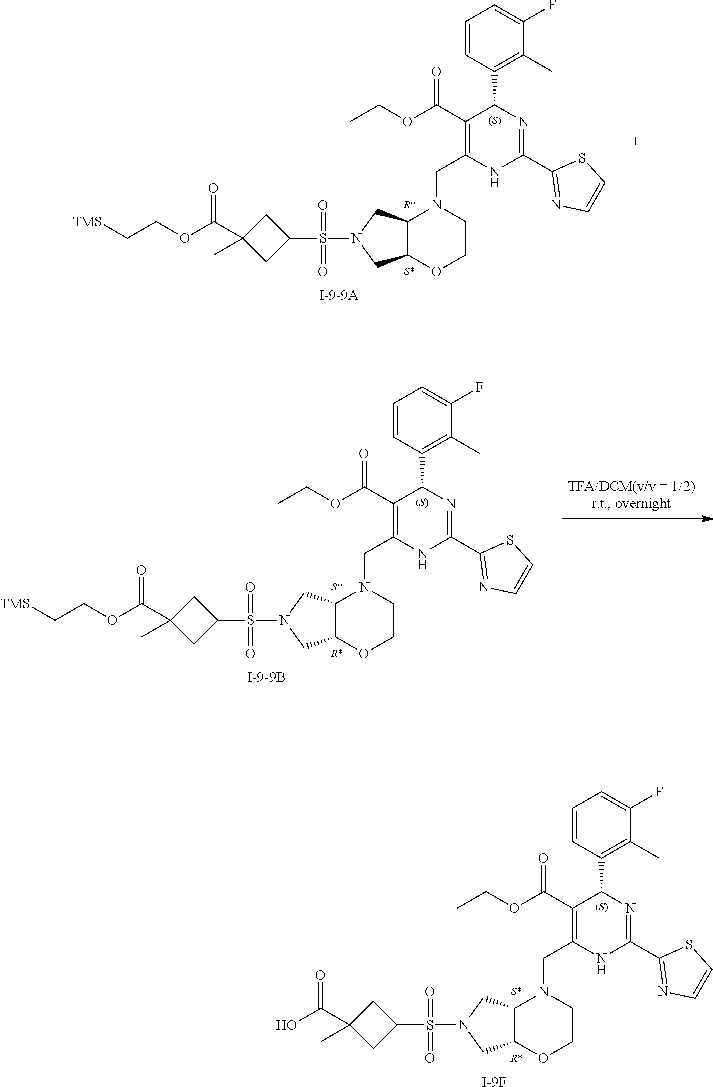

### Intermediate I-9-2: 1-Methyl-3-oxocyclobutanecarboxylic acid

To a solution of ethyl 1-methyl-3-oxocyclobutanecarboxylate I-9-1 (4.00 g, 25.6 mmol) in methanol (50 mL) and water (10 mL) was added lithium hydroxide monohydrate (2.15 g, 51.2 mmol). After stirred at room temperature overnight, the mixture was acidified with 2 M hydrochloride aqueous solution to pH˜2 and then concentrated under reduced pressure to give a residue, which was dissolved in water (50 mL) and extracted with ethyl acetate (30 mL) for three times. The combined organic layers were dried over Na₂SO₄₍ₛ₎ and concentrated to afford the title compound (4.10 g, 90% purity, 100% yield) as yellow oil.

¹H NMR (400 MHz, CDCl₃) δ 3.67-3.62 (m, 2H), 2.99-2.93 (m, 2H), 1.62 (s, 3H).

### Intermediate I-9-3: 2-(Trimethylsilyl)ethyl 1-methyl-3-oxocyclobutanecarboxylate

To a solution of 1-methyl-3-oxocyclobutanecarboxylic acid I-9-2 (4.10 g, purity 90%, 28.8 mmol) and 2-(trimethylsilyl)ethanol (3.75 g, 31.7 mmol), N,N-dimethylpyridin-4-amine (3.50 g, 28.6 mmol) in tetrahydrofuran (100 mL) was added 1-ethyl-3-(3-dimethylaminopropyl)carbodiimide hydrochloride (8.30 g, 43.2 mmol) under nitrogen. After stirred at room temperature overnight, the mixture was quenched with water (50 mL), then concentrated under reduced pressure to remove the volatile, extracted with ethyl acetate (80 mL) for three times. The combined organic layers were washed with 2M hydrochloride aqueous solution (100 mL), dried over Na₂SO₄₍ₛ₎ and concentrated to afford the title compound (5.10 g, 80% purity, 100% yield) as yellow oil.

¹H NMR (400 MHz, CDCl₃) δ 4.21-4.17 (m, 2H), 3.54-3.49 (m, 2H), 2.85-2.80 (m, 2H), 1.52 (s, 3H), 0.99-0.95 (m, 2H), 0.07 (s, 9H).

### Intermediate I-9-4: 2-(Trimethylsilyl)ethyl 3-hydroxy-1-methylcyclobutanecarboxylate (a Mixture of 2 Stereoisomers)

To a solution of 2-(trimethylsilyl)ethyl 1-methyl-3-oxocyclobutanecarboxylate I-9-3 (5.10 g, 80% purity, 17.9 mmol) in tetrahydrofuran (100 mL) and water (10 mL) was added portionwise sodium tetrahydroborate (2.70 g, 71.5 mmol) at room temperature. After stirred at room temperature overnight, the mixture was quenched with water (20 mL) and then concentrated under reduced pressure to give a residue. The residue was taken up with water (50 mL) and extracted with ethyl acetate (50 mL) for three times. The combined organic layers were dried over Na₂SO₄₍ₛ₎ and concentrated to afford the title compound (3.80 g, 90% purity, 100% yield) as yellow oil.

¹H NMR (400 MHz, CDCl₃) δ 4.38-4.22 (m, 1H), 4.16-4.10 (m, 2H), 3.71-3.67 (m, 1H), 2.80-2.71 (m, 1H), 2.32-2.21 (m, 2H), 1.85-1.77 (m, 1H), 1.35 (s, 1.5H), 1.30 (s, 1.5H), 0.97-0.89 (m, 2H), 0.07 (s, 9H).

### Intermediate I-9-5: 2-(Trimethylsilyl)ethyl 1-methyl-3-(tosyloxy)cyclobutanecarboxylate (a Mixture of 2 Stereoisomers)

To a solution of 2-(trimethylsilyl)ethyl 3-hydroxy-1-methylcyclobutanecarboxylate I-9-4 (3.70 g, 90% purity, 14.5 mmol) and N,N-dimethylpyridin-4-amine (2.90 g, 23.7 mmol) in dichloromethane (100 mL) was added 4-methylbenzene-1-sulfonyl chloride (3.3 g, 17.3 mmol) under nitrogen. After stirred at room temperature overnight, the reaction solution was washed with water (40 mL), dried over Na₂SO₄₍ₛ₎ and concentrated under reduced pressure to give a residue. The residue was purified by silica gel column chromatography (petroleum ether:ethyl acetate=8:1) to afford the title compound (3.10 g, 95% purity, 48% yield) as light yellow oil.

¹H NMR (400 MHz, CDCl₃) δ 7.74 (d, J=8.4 Hz, 2H), 7.30 (d, J=8.0 Hz, 2H), 4.87-4.83 (m, 1H), 4.14-4.10 (m, 1H), 2.72-2.66 (m, 0.8H), 2.56-2.52 (m, 1.2H), 2.42 (s, 3H), 2.17-2.11 (m, 1.2H), 2.08-2.03 (m, 0.8H), 1.33 (s, 1H), 1.28 (s, 2H), 0.95-0.91 (m, 2H), 0.01 (s, 9H).

### Intermediate I-9-6: 2-(Trimethylsilyl)ethyl 3-(acetylthio)-1-methylcyclobutanecarboxylate (a Mixture of 2 Stereoisomers)

To a solution of 2-(trimethylsilyl)ethyl 1-methyl-3-(tosyloxy)cyclobutanecarboxylate I-9-5 (3.10 g, 95% purity 7.26 mmol) in N,N-dimethylformamide (70 mL) was added potassium thioacetate (3.00 g, 26.3 mmol) at room temperature. After stirred at 100° C. for 6 hours, the reaction mixture was cooled down and poured into water (200 mL) and extracted with ethyl acetate (80 mL) for three times. The combined organic layers were washed with water (100 mL) for three times, dried over Na₂SO₄₍ₛ₎ and concentrated to afford the title compound (2.30 g, 90% purity, 99% yield) as yellow oil.

¹H NMR (400 MHz, CDCl₃) δ 4.27-4.21 (m, 2H), 4.19-4.08 (m, 1H), 3.06-3.00 (m, 1H), 2.59-2.50 (m, 1H), 2.41-2.36 (m, 1H), 2.32 (s, 3H), 2.04-1.99 (m, 1H), 1.50 (s, 1.2H), 1.44 (s, 1.8H), 1.08-1.01 (m, 2H), 0.07 (s, 9H).

### Intermediate I-9-7: 2-(Trimethylsilyl)ethyl 3-(chlorosulfonyl)-1-methylcyclobutanecarboxylate (a Mixture of 2 Stereoisomers)

To a solution of 2-(trimethylsilyl)ethyl 3-(acetylthio)-1-methylcyclobutane carboxylate I-9-6 (1.10 g, 90% purity 7.52 mmol) in acetonitrile (50 mL) was added 2M hydrochloride aqueous solution (0.2 mL) and 1-chloropyrrolidine-2,5-dione (1.35 g, 10.1 mmol) at 0° C. After stirred at 0° C. for 1 hour, the resulting mixture was concentrated under reduced pressure and the obtained crude product was poured in water (30 mL), extracted with ethyl acetate (30 mL) for three times The combined organic layers were washed with water (20 mL) for three times, dried over Na₂SO₄₍ₛ₎ and concentrated to give a residue, which was purified by silica gel column chromatography (petroleum ether:ethyl acetate=10:1) to afford the title compound (820 mg, 95% purity, 73% yield) as light yellow oil.

¹H NMR (400 MHz, CDCl₃) δ 4.48-4.33 (m, 1H), 4.26-4.19 (m, 2H), 3.16-3.10 (m, 0.8H), 2.97-2.91 (m, 1.2H), 2.63-2.57 (m, 1.2H), 2.41-2.36 (m, 0.8H), 1.48 (s, 3H), 1.04-0.99 (m, 2H), 0.07 (s, 9H).

### Intermediate I-9-8: (S)-(cis)-Ethyl 4-(3-fluoro-2-methylphenyl)-6-((hexahydropyrrolo[3,4-b][1,4]oxazin-4 (4aH)-yl)methyl)-2-(thiazol-2-yl)-1,4-dihydropyrimidine-5-carboxylate(a Mixture of 2 Stereoisomers)

To a solution of (cis)-tert-butyl 4-(((S)-5-(ethoxycarbonyl)-6-(3-fluoro-2-methylphenyl)-2-(thiazol-2-yl)-3,6-dihydropyrimidin-4-yl)methyl)hexahydropyrrolo[3,4-b][1,4]oxazine-6(2H)-carboxylate I-5-1 (200 mg, 90% purity, 0.307 mmol) in dichloromethane (3 mL) was added trifluoroacetic acid (3 mL). After stirred at room temperature for 3 hours, saturated sodium bicarbonate aqueous solution was added to adjust pH˜7. The mixture was extracted with dichloromethane (10 mL) twice. The combined extracts were dried over Na₂SO₄₍ₛ₎, filtered and concentrated to give the title compound (153 mg, 90% purity from ¹H NMR, 92% yield) as yellow solids.

LC-MS (ESI): R_{T}=1.72 min, mass calcd. for C₂₄H₂₈FN₅O₃S 485.6, m/z found 486.6 [M+H]⁺. ¹H NMR (400 MHz, CDCl₃) δ 9.52 (s, 1H), 7.82-7.81 (m, 1H), 7.42-7.41 (m, 1H), 7.09-7.04 (m, 1H), 7.00-6.97 (m, 1H), 6.92-6.88 (m, 1H), 6.02 (d, J=6.8 Hz, 1H), 4.29-4.17 (m, 2H), 4.12-3.77 (m, 5H), 3.41-3.30 (m, 2H), 3.16-3.00 (m, 3H), 2.92-2.78 (m, 1H), 2.58-2.54 (m, 3H), 2.45-2.42 (m, 0.5H), 1.12 (t, J=7.2 Hz, 3H).

### Intermediate I-9-9: (S)-(cis)-Ethyl 4-(3-fluoro-2-methylphenyl)-6-((6-((3-methyl-3-((2-(trimethylsilyl)ethoxy)carbonyl)cyclobutypsulfonyl)hexahydropyrrolo[3,4-b][1,4]oxazin-4(4aH)-yl)methyl)-2-(thiazol-2-yl)-1,4-dihydropyrimidine-5-carboxylate (a Mixture of 4 Stereoisomers)

To a solution of (S)-(cis)-ethyl 4-(3-fluoro-2-methylphenyl)-6-((hexahydropyrrolo[3,4-b][1,4]oxazin-4(4aH)-yl)methyl)-2-(thiazol-2-yl)-1,4-dihydropyrimidine-5-carboxylate I-9-8 (153 mg, 90% purity, 0.284 mmol) in 1,4-dioxane (5 mL) was added 2-(trimethylsilyl)ethyl 3-(chlorosulfonyl)-1-methylcyclobutanecarboxylate I-9-7 (273 mg, 90% purity, 0.785 mmol) and triethylamine (96 mg, 0.949 mmol). After stirred at room temperature for 4 hours, the solution was concentrated to give a residue, which was purfied by silica gel column chromtography (ethyl acetate:petroleum ether=1:3) to afford the desired compound (138 mg, 94% purity, 60% yield) as yellow solids.

LC-MS (ESI): R_{T}=2.06 min, mass calcd. for C₃₅H₄₈FN5O7S2Si 762.0, m/z found 762.8 [M+H]⁺.

### Intermediate I-9-9A and I-9-9B: (4a/?*,7aS*)—(S)-Ethyl 4-(3-fluoro-2-methylphenyl)-6-((6-((3-methyl-3-((2-(trimethylsilyl)ethoxy)carbonyl)cyclobutypsulfonyl)hexahydropyrrolo[3,4-b][1,4]oxazin-4(4aH)-yl)methyl)-2-(thiazol-2-yl)-1,4-dihydropyrimidine-5-carboxylate (a Mixture of 2 Stereoisomers) and (4aS*,7aR*)—(S)-ethyl 4-(3-fluoro-2-methylphenyl)-6-((6-((3-methyl-3-((2-(trimethylsilyl)ethoxy)carbonyl)cyclobutypsulfonyl)hexahydropyrrolo[3,4-b][1,4]oxazin-4(4aH)-yl)methyl)-2-(thiazol-2-yl)-1,4-dihydropyrimidine-5-carboxylate (a Mixture of 2 Stereoisomers)

A racemic mixture of (S)-(cis)-ethyl 4-(3-fluoro-2-methylphenyl)-6-((6-((3-methyl-3-((2-(trimethylsilyl)ethoxy)carbonyl)cyclobutypsulfonyl)hexahydropyrrolo[3,4-b][1,4]oxazin-4-yl)methyl)-2-(thiazol-2-yl)-1,4-dihydropyrimidine-5-carboxylate I-9-9 (120 mg, 94% purity, 0.148 mmol) was separated by chiral Prep. HPLC (Column: Chiralpak IC 5 μm 20*250 mm; Mobile Phase: Hex:EtOH:DEA=60:40:0.3 at 13 mL/min; Temp: 30° C.; Wavelength: 214 nm) to give the title compounds I-9-9A (40 mg, 98.7% purity, 35% yield, 100% stereopure) and I-9-9B (30 mg, 99.5% purity, 26% yield, 99.8% stereopure) as yellow solids.

Intermediate I-9-9A: LC-MS (ESI): R_{T}=2.226 min, mass calcd. for C₃₅H₄₈FN₅O₇S₂Si 762.0, m/z found 762.3 [M+H]⁺. Chiral analysis (Column: Chiralpak IC 5 μm 4.6*250 mm; Mobile Phase: Hex:EtOH:DEA=60:40:0.2 at 1 mL/min; Temp: 30° C.; Wavelength: 254 nm, R_{T}=9.312 min).

Intermediate I-9-9B: LC-MS (ESI): R_{T}=2.221 min, mass calcd. for C₃₅H₄₈FN5O7S2Si 762.0, m/z found 762.2 [M+H]⁺. Chiral analysis (Chiralpak IC 5 μm 4.6*250 mm; Mobile Phase: Hex:EtOH:DEA=60:40:0.2 at 1 mL/min; Temp: 30° C.; Wavelength: 254 nm, R_{T}=12.250 min).

### Compound I-9F: (4aS*,7aR*)—(S)-3-((4-((5-(Ethoxycarbonyl)-6-(3-fluoro-2-methylphenyl)-2-(thiazol-2-yl)-3,6-dihydropyrimidin-4-yl)methyphexahydropyrrolo[3,4-b][1,4]oxazin-6(2H)-yl)sulfonyl)-1-methylcyclobutanecarboxylic acid (a Mixture of 2 Stereoisomers)

To a solution of (4aS*,7aR*)—(S)-ethyl 4-(3-fluoro-2-methylphenyl)-6-((6-((3-methyl-3-((2-(trimethylsilyl)ethoxy)carbonyl)cyclobutypsulfonyl)hexahydropyrrolo[3,4-b][1,4]oxazin-4(4aH)-yl)methyl)-2-(thiazol-2-yl)-1,4-dihydropyrimidine-5-carboxylate I-9-9B (30 mg, 99.5% purity, 0.039 mmol) in dichloromethane (2 mL) was added trifluoroacetic acid (1 mL) at room temperature. After stirred at room temperature overnight, the mixture was concentrated and purified by C18 column (acetonitrile:water (+0.1% ammonium bicarbonate)=50% to 95%) to give the title compound (3.5 mg, 98% purity, 13% yield) as yellow solids.

LC-MS (ESI): R_{T}=3.498 min, mass calcd. for C₃₀H₃₆FN₅O₇S 3.746 min, m/z found 662.2 [M+H]⁺. ¹H NMR (400 MHz, DMSO-d₆) δ 9.48 (s, 1H), 8.00 (d, J=3.2 Hz, 1H), 7.93 (d, J=3.2 Hz, 1H), 7.18-7.15 (m, 1H), 7.04-7.00 (m, 2H), 5.90 (s, 0.2H), 5.89 (s, 0.8H), 4.15-3.81 (m, 7H), 3.61-3.42 (m, 4H), 3.25-3.22 (m, 1H), 2.89-2.58 (m, 4H), 2.45 (s, 3H), 2.40-2.19 (m, 3H), 1.35 (s, 0.5H), 1.30 (s, 2H), 1.23 (s, 0.5H), 1.06 (t, J=6.8 Hz, 3H).

### Compound I-10A: 3-((4aR*,8aS*)-4-(((S)-5-(Ethoxycarbonyl)-6-(3-fluoro-2-methylphenyl)-2-(thiazol-2-yl)-3,6-dihydropyrimidin-4-yl)methyl)hexahydro-2H-pyrido[4,3-b][1,4]oxazin-6(7H)-yl)-2,2-dimethylpropanoic acid (a Single Stereoisomer)

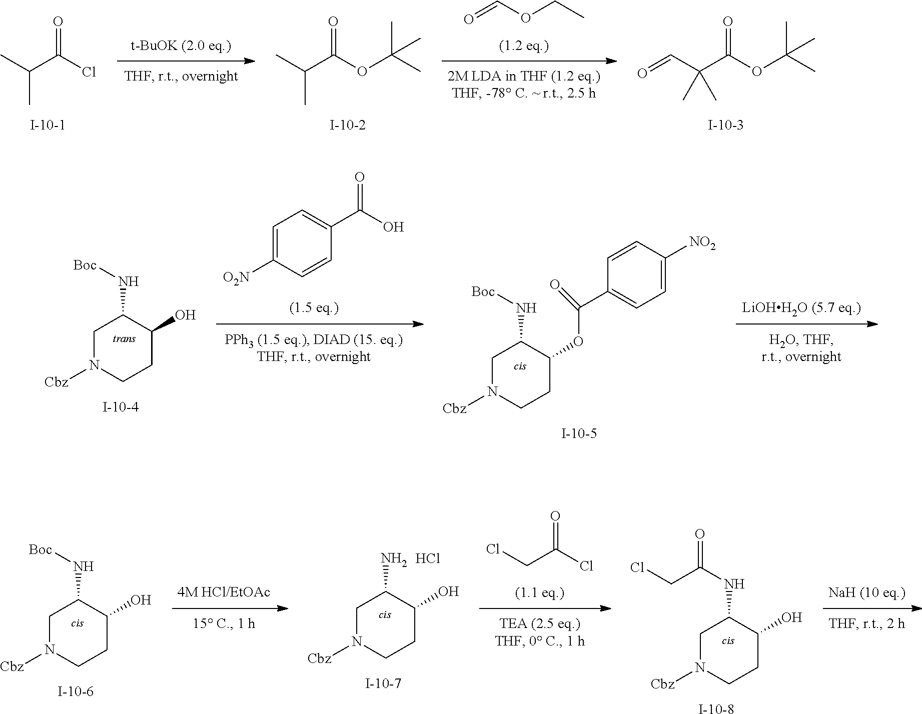

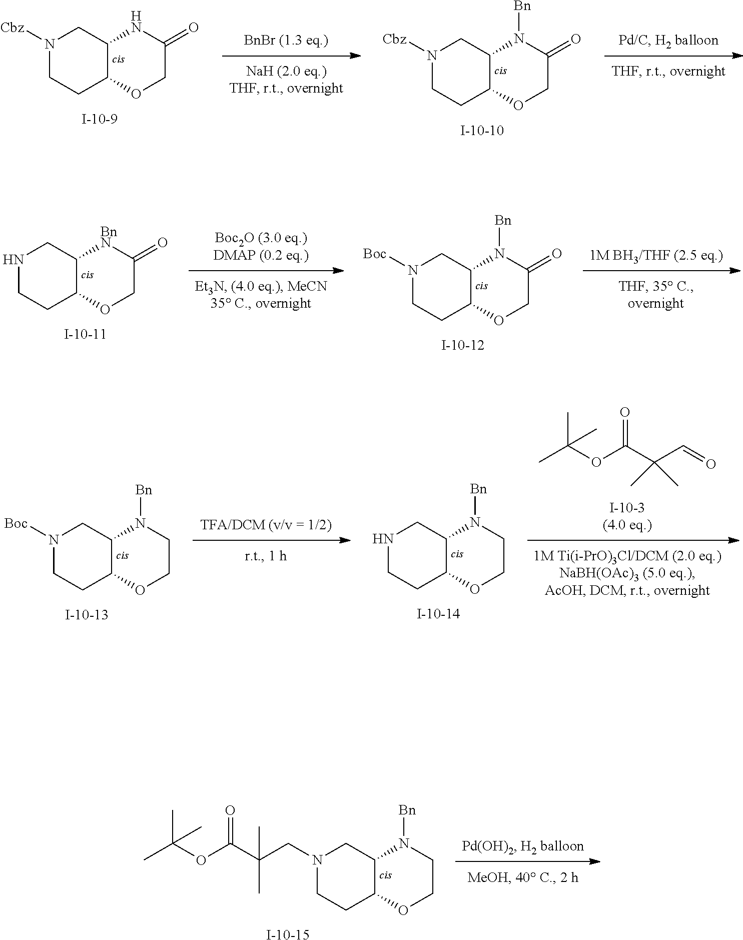

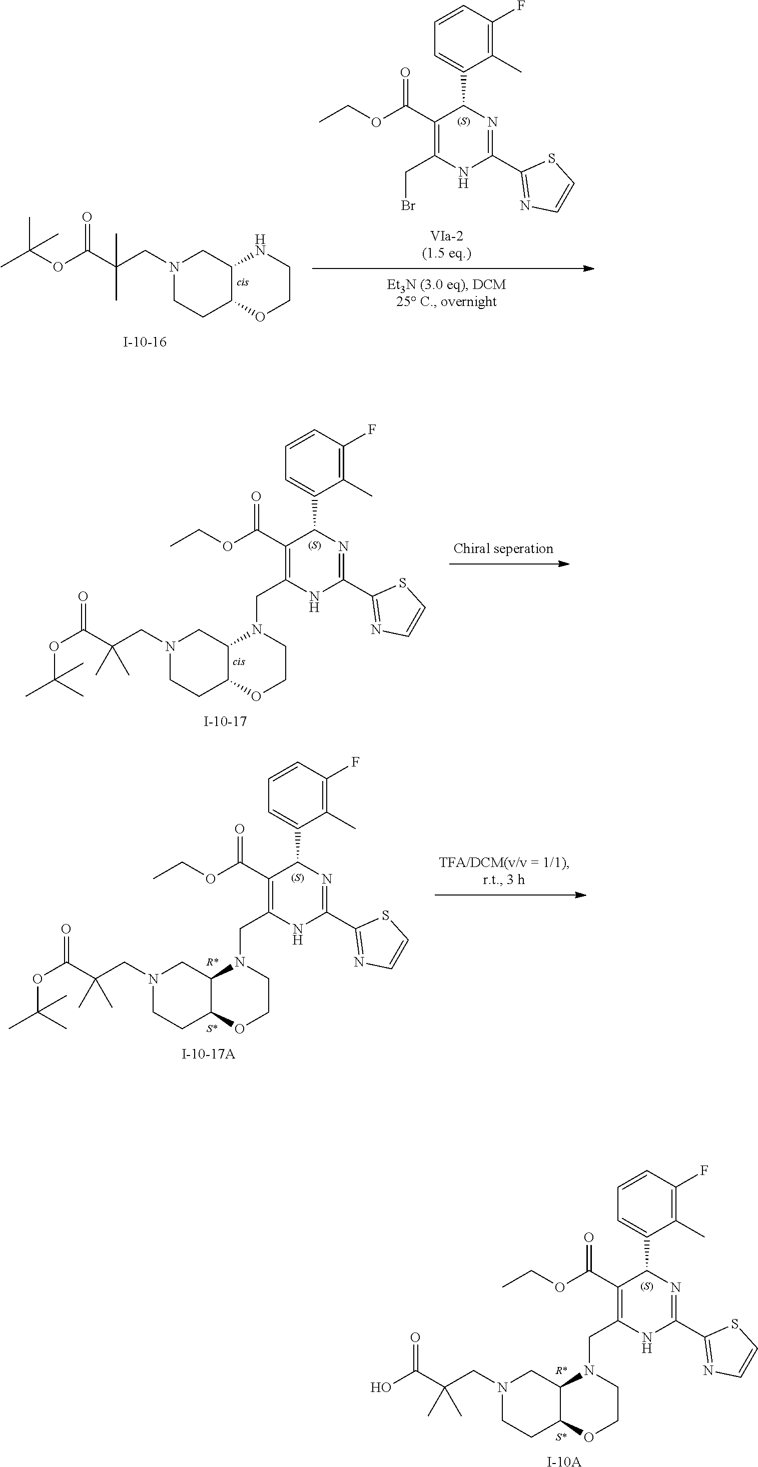

### Intermediate I-10-2: tert-Butyl isobutyrate

To a solution of isobutyryl chloride I-10-1 (70.0 g, 657 mmol) in tetrahydrofuran (100 mL) was added potassium tert-butoxide (150 g, 1.34 mol) at 0° C. After stirred at room temperature overnight, the mixture was quenched with saturated ammonium chloride aqueous solution (120 mL) at 0° C. under nitrogen atmosphere and extracted with ethyl acetate (80 mL) twice. The combined organic layers were concentrated under reduced pressure distillation to afford the title compound (77.0 g, 90% purity from ¹H NMR, 73% yield) as colorless oil.

¹H NMR (400 MHz, CDCl₃) δ 2.44-2.37 (m, 1H), 1.42 (s, 9H), 1.10-1.08 (m, 6H).

### Intermediate I-10-3: tert-Butyl 2,2-dimethyl-3-oxopropanoate

To a solution of tert-butyl isobutyrate I-10-2 (95.0 g, 95% purity, 626 mmol) in tetrahydrofuran (900 mL) was added 2 M lithium diisopropylamide in tetrahydrofuran (376 mL, 752 mmol) at −78° C. under nitrogen atmosphere. The mixture was stirred at −78° C. for 0.5 hour, and then ethyl formate (56.0 g, 756 mmol) was added. The reaction mixture was stirred at room temperature for 2 hours. The mixture was poured into water (1000 mL) and extracted with ethyl acetate (400 mL) twice. The combined organic phases were washed with brine (200 mL), dried over Na₂SO₄₍ₛ₎ and filtered. The filtrate was concentrated in vacuum to give the desired compound (152 g, 70% purity from ¹H NMR, 98% yield) as yellow oil.

¹H NMR (400 MHz, CDCl₃) δ 8.63 (s, 1H), 1.46 (s, 9H), 1.30 (s, 6H).

### Intermediate I-10-5: (cis)-Benzyl 3-((tert-butoxycarbonyl)amino)-4-((4-nitrobenzoyl)oxy)piperidine-1-carboxylate (a Mixture of 2 Stereoisomers)

To a solution of (trans)-benzyl 3-((tert-butoxycarbonyl)amino)-4-hydroxypiperidine-1-carboxylate I-10-4 (15.3 g, 43.7 mmol), triphenylphosphine (17.2 g, 65.6 mmol) and 4-nitrobenzoic acid (11.0 g, 65.6 mmol) in tetrahydrofuran (300 mL) was added diisopropyl azodicarboxylate (13.2 g, 65.6 mmol) dropwise at 0° C. After stirred at room temperature overnight under nitrogen atmosphere, the reaction mixture was poured into water (200 mL) and extracted with ethyl acetate (150 mL) for three times. The combined organic phases were washed with brine (150 mL), dried over Na₂SO₄₍ₛ₎ and filtered. The filtrate was concentrated and purified by silica gel column chromatography (petroleum ether:ethyl acetate=6:1) to give the title compound (25.0 g, crude) as white solids.

LC-MS (ESI): R_{T}=1.902 min, mass calcd. for C₂₅H₂₉N₃O₈ 499.2, m/z found 444.1 [M−56+H]⁺.

### Intermediate I-10-6: (cis)-Benzyl 3-((tert-butoxycarbonyl)amino)-4-hydroxypiperidine-1-carboxylate (a Mixture of 2 Stereoisomers)

To a solution of (cis)-benzyl 3-((ter t-butoxycarbonyl)amino)-4-((4-nitrobenzoyl)oxy)piperidine-1-carboxylate I-10-5 (25.0 g, crude, ˜43.7 mmol) in tetrahydrofuran (250 mL) and water (150 mL) was added lithium hydroxide monohydrate (10.5 g, 250 mmol). After stirred at room temperature overnight, the reaction mixture was diluted with water (150 mL), removed tetrahydrofuran in vacuo and extracted with ethyl acetate (150 mL) for three times. The combined organic layers were washed with brine (150 mL), dried over Na₂SO₄₍ₛ₎ and filtered. The filtrate was concentrated and purified by silica gel column chromatography (petroleum ether:ethyl acetate=3:1) to give the title compound (15.0 g, 98% yield) as white solids.

¹H NMR (400 MHz, CDCl₃) δ 7.35-7.26 (m, 5H), 5.18-5.09 (m, 2H), 4.93 (br s, 1H), 3.98 (s, 1H), 3.80-3.32 (m, 5H), 2.52-2.43 (m, 1H), 1.74-1.65 (m, 2H), 1.44 (s, 9H).

### Intermediate I-10-7: (cis)-Benzyl 3-amino-4-hydroxypiperidine-1-carboxylate hydrochloride (a Mixture of 2 Stereoisomers)

To a solution of (cis)-benzyl 3-((tert-butoxycarbonyl)amino)-4-hydroxypiperidine-1-carboxylate I-10-6 (6.80 g, 19.4 mmol) in 4 M hydrochloride in ethyl acetate (60 mL) was stirred at 15° C. for 1 hour. The reaction mixture was concentrated in vacuo to give the title compound (5.50 g, 99% yield) as white solids.

¹H NMR (400 MHz, DMSO-d₆) δ 8.19-8.14 (m, 3H), 7.39-7.31 (m, 5H), 5.69 (s, 1H), 5.08 (s, 2H), 4.00 (s, 1H), 3.79 (d, J=10.4 Hz, 1H), 3.54-3.49 (m, 1H), 3.19 (s, 1H), 1.68-1.66 (m, 2H).

### Intermediate I-10-8: (cis)-Benzyl 3-(2-chloroacetamido)-4-hydroxypiperidine-1-carboxylate (a Mixture of 2 Stereoisomers)

To a solution of (cis)-benzyl 3-amino-4-hydroxypiperidine-1-carboxylate hydrochloride I-10-7 (5.50 g, 19.2 mmol), triethylamine (4.86 g, 48.1 mmol) in tetrahydrofuran (70 mL) was added 2-chloroacetyl chloride (2.40 g, 21.1 mmol) dropwised at 0° C. After stirred at 0° C. for 1 hour, the reaction mixture was diluted with ethyl acetate (100 mL), washed with brine (100 mL) twice. The separated organic layer was dried over Na₂SO₄₍ₛ₎ and filtered. The filtrate was concentrated and purified by silica gel column chromatography (petroleum ether:ethyl acetate=1:1) to give the title compound (4.60 g, 73% yield) as white solids.

¹H NMR (400 MHz, DMSO-d₆) δ 7.98 (d, J=10.4 Hz, 1H), 7.40-7.34 (m, 5H), 5.15 (d, J=5.2 Hz, 1H), 5.10 (s, 2H), 4.14 (s, 2H), 3.86-3.54 (m, 4H), 3.40-3.35 (m, 1H), 1.70-1.63 (m, 2H).

### Intermediate I-10-9: (cis)-Benzyl 3-oxohexahydro-2H-pyrido[4,3-b][1,4]oxazine-6(7H)-carboxylate (a Mixture of 2 Stereoisomers)

A solution of (cis)-benzyl 3-(2-chloroacetamido)-4-hydroxypiperidine-1-carboxylate I-10-8 (4.10 g, 12.5 mmol) in tetrahydrofuran (2000 mL) under nitrogen atmosphere at 0° C. was added 60% wt. sodium hydride in mineral oil (5.00 g, 125 mmol). After stirred at room temperature for 2 hours, the reaction mixture was poured into water (500 mL) and extracted with ethyl acetate (500 mL) twice. The combined organic phases were washed with brine (200 mL), dried over Na₂SO₄₍ₛ₎ and filtered. The filtrate was concentrated and purified by C18 column (acetonitrile:water=30% to 95%) to give the title compound (1.74 g, 43% yield) as white solids.

¹H NMR (400 MHz, DMSO-d₆) δ 8.16 (s, 1H), 7.40-7.31 (m, 5H), 5.08 (s, 2H), 4.10-3.98 (m, 3H), 3.92-3.91 (m, 1H), 3.82-3.78 (m, 1H), 3.26-3.24 (m, 1H), 3.93-3.88 (m, 2H), 1.79-1.74 (m, 2H).

### Intermediate I-10-10: (cis)-Benzyl 4-benzyl-3-oxohexahydro-2H-pyrido[4,3-b][1,4]oxazine-6(7H)-carboxylate (a Mixture of 2 Stereoisomers)

A solution of (cis)-benzyl 3-oxohexahydro-2H-pyrido[4,3-b][1,4]oxazine-6 (7H)-carboxylate I-10-9 (1.64 g, 5.66 mmol) in tetrahydrofuran (20 mL) under nitrogen atmosphere at 0° C. was added 60% wt. sodium hydride in mineral oil (450 mg, 11.4 mmol). Then benzyl bromide (1.27 g, 7.40 mmol) was added. After stirred at room temperature overnight, the reaction mixture was poured into water (100 mL) and extracted with ethyl acetate (100 mL) twice. The combined organic phases were washed with brine (100 mL), dried over Na₂SO₄₍ₛ₎ and filtered. The filtrate was concentrated and purified by silica gel column chromatography (petroleum ether:ethyl acetate=10:1) to give the title compound (1.47 g, 68% yield) as white solids.

LC-MS (ESI): R_{T}=1.722 min, mass calcd. for C₂₂H₂₄N₂O₄ 380.2, m/z found 381.2 [M+H]⁺.

### Intermediate I-10-11: (cis)-4-Benzylhexahydro-2H-pyrido[4,3-b][1,4]oxazin-3(4H)-one (a Mixture of 2 Stereoisomers)

To a solution of (cis)-benzyl 4-benzyl-3-oxohexahydro-2H-pyrido[4,3-b][1,4]oxazine-6(7H)-carboxylate I-10-10 (1.50 g, 3.95 mmol) in tetrahydrofuran (20 mL) was added 10% palladium on activated carbon wt. (1.00 g) at room temperature. After stirred at room temperature under hydrogen atmosphere balloon overnight, the reaction mixture was filtered and concentrated to afford the title compound (1.0 g, 90% purity from ¹H NMR, 93% yield) as light yellow oil.

¹H NMR (400 MHz, CDCl₃) δ 7.36-7.25 (m, 5H), 4.94-4.90 (m, 1H), 4.26-4.17 (m, 3H), 3.95 (d, J=1.6 Hz, 1H), 3.47-3.42 (m, 1H), 3.21-3.16 (m, 1H), 3.11-3.07 (m, 1H), 2.67-2.61 (m, 2H), 1.76-1.67 (m, 2H), 1.06 (t, J=6.8 Hz, 1H).

### Intermediate I-10-12: (cis)-tert-Butyl 4-benzyl-3-oxohexahydro-2H-pyrido[4,3-b][1,4]oxazine-6(7H)-carboxylate (a Mixture of 2 Stereoisomers)

To a solution of (cis)-4-benzylhexahydro-2H-pyrido[4,3-b][1,4]oxazin-3(4H)-one I-10-11 (550 mg, 90% purity, 2.01 mmol) in acetonitrile (10 mL) was added triethylamine (800 mg, 7.91 mmol), di-tert-butyl dicarbonate (1.32 g, 6.05 mmol) and 4-dimethylaminopyridine (50 mg, 0.410 mmol). After stirred at 35° C. overnight, the mixture was concentrated under reduced pressure to give a residue, which was purified by C18 column (acetonitrile:water=05% to 95%) to give the title compound (700 mg, 90% purity from ¹H NMR, 90% yield) as yellow oil.

¹H NMR (400 MHz, CDCl₃) δ 7.36-7.28 (m, 5H), 5.34-5.24 (m, 1H), 4.41-3.88 (m, 6H), 3.14-2.93 (m, 3H), 1.87-1.83 (m, 1H), 1.73-1.67 (m, 1H), 1.45 (s, 9H).

### Intermediate I-10-13: (cis)-tert-Butyl 4-benzylhexahydro-2H-pyrido[4,3-b][1,4]oxazine-6(7H)-carboxylate (a Mixture of 2 Stereoisomers)

To a solution of (cis)-tert-butyl 4-benzyl-3-oxohexahydro-2H-pyrido[4,3-b][1,4]oxazine-6(7H)-carboxylate I-10-12 (700 mg, 90% purity, 1.82 mmol) in tetrahydrofuran (10 mL) was added 1 M borane-tetrahydrofuran complex in tetrahydrofuran (4.6 mL, 4.6 mmol) at 0° C. slowly. After stirred at 35° C. overnight, the mixture was quenched with 1 M hydrochloride aqueous solution, diluted with water (20 mL) and extracted with ethyl acetate (20 mL) twice. The combined organic layers was washed by brine (20 mL), dried over Na₂SO₄(s), filtered and concentrated under reduced pressure to give the desired product (720 mg, 78% purity, 93% yield) as yellow oil.

LC-MS (ESI): R_{T}=1.904 min, mass calcd. for C₁₉H₂₈N₂O₃ 332.2, m/z found 333.2 [M+H]⁺.

### Intermediate I-10-14: (cis)-4-Benzyloctahydro-2H-pyrido[4,3-b][1,4]oxazine (a Mixture of 2 Stereoisomers)

To a solution of (cis)-tert-butyl 4-benzylhexahydro-2H-pyrido[4,3-b][1,4]oxazine-6(7H)-carboxylate I-10-13 (720 mg, 78% purity, 1.69 mmol) in dichloromethane (10 mL) was added trifluoroacetic acid (5 mL). After stirred at room temperature for 1 hour, the mixture was concentrated under reduced pressure to give a residue, which was purified by C18 column (acetonitrile:water (+0.02% ammonium bicarbonate)=05% to 95%) to give the title compound (256 mg, 90% purity from ¹H NMR, 59% yield) as yellow oil.

¹H NMR (400 MHz, CDCl₃) δ 7.35-7.29 (m, 4H), 7.26-7.22 (m, 1H), 3.90-3.83 (m, 2H), 3.75-3.62 (m, 3H), 3.24-3.18 (m, 1H), 2.94-2.67 (m, 5H), 2.36-2.32 (m, 1H), 1.89-1.86 (m, 1H), 1.70-1.67 (m, 1H).

### Intermediate I-10-15: (cis)-tert-Butyl 3-(4-benzylhexahydro-2H-pyrido[4,3-b][1,4]oxazin-6(7H)-yl)-2,2-dimethylpropanoate (a Mixture of 2 Stereoisomers)

To a mixture of (cis)-4-benzyloctahydro-2H-pyrido[4,3-b][1,4]oxazine I-10-14 (150 mg, 90% purity, 0.581 mmol) and tert-butyl 2,2-dimethyl-3-oxopropanoate I-10-3 (570 mg, 70% purity, 2.32 mmol) in anhydrous dicholormethane (5 mL) was added 1 M chlorotriisopropoxytitanium(IV) in dicholormethane (1.2 mL, 1.2 mmol) at room temperature under nitrogen atmosphere. After the addition, the mixture was stirred at room temperature for 0.5 hour. Sodium triacetoxyborohydride (600 mg, 2.83 mmol) was added, followed by the addition of acetic acid (0.2 mL). After stirred at room temperature overnight, the reaction mixture was quenched with saturated sodium bicarbonate aqueous solution (20 mL) and extracted with dichloromethane (20 mL) twice. The combined organic layers were washed with brine (20 mL), dried over Na₂SO₄₍ₛ₎ and filtered. The filtrate was concentrated to give a residue, which was purified by C18 column (acetonitrile:water=05% to 95%) to give the title compound (115 mg, 95% purity, 48% yield) as yellow oil.

LC-MS (ESI): R_{T}=2.14 min, mass calcd. for C₂₃H₃₆N₂O₃ 388.3, m/z found 389.5 [M+H]⁺.

### Intermediate I-10-16: (cis)-tert-Butyl 3-(hexahydro-2H-pyrido[4,3-b][1,4]oxazin-6(7H)-yl)-2,2-dimethylpropanoate (a Mixture of 2 Stereoisomers)

To a solution of (cis)-tert-butyl 3-(4-benzylhexahydro-2H-pyrido[4,3-b][1,4]oxazin-6(7H)-yl)-2,2-dimethylpropanoate I-10-15 (115 mg, 95% purity, 0.281 mmol) in methanol (5 mL) was added 20% palladium hydroxide wt. (10 mg). After stirred at 40° C. under hydrogen atmosphere balloon for 2 hours, the mixture was filtered and the filtrate was concentrated under reduced pressure to give the title product (84 mg, 90% purity from ¹H NMR, 90% yield) as colorless oil.

¹H NMR (400 MHz, CDCl₃) δ 3.87-3.79 (m, 1H), 3.65-3.49 (m, 2H), 3.11-3.03 (m, 1H), 2.90-2.83 (m, 2H), 2.70-2.60 (m, 1H), 2.53-2.36 (m, 5H), 1.93-1.85 (m, 0.6H), 1.75-1.65 (m, 1.4H), 1.44 (s, 9H), 1.10 (s, 3H), 1.09 (s, 3H).

### Intermediate I-10-17: (cis)-(S)-Ethyl 6-((6-(3-(tert-butoxy)-2,2-dimethyl-3-oxopropyl)hexahydro-2H-pyrido[4,3-b][1,4]oxazin-4(3H)-yl)methyl)-4-(3-fluoro-2-methylphenyl)-2-(thiazol-2-yl)-1,4-dihydropyrimidine-5-carboxylate (a Mixture of 2 Stereoisomers)

To a solution of (cis)-tert-butyl 3-(hexahydro-2H-pyrido[4,3-b][1,4]oxazin-6 (7H)-yl)-2,2-dimethylpropanoate I-10-16 (80 mg, 90% purity, 0.242 mmol) and (S)-ethyl 6-(bromomethyl)-4-(3-fluoro-2-methylphenyl)-2-(thiazol-2-yl)-1,4-dihydropyrimidine-5-carboxylate VIa-2 (166 mg, 95% purity, 0.360 mmol) in dichloromethane (4 mL) was added triethylamine (74 mg, 0.733 mmol). After stirred at 25° C. overnight, the mixture was concentrated to give a residue, which was purified by C18 column (acetonitrile:water=05% to 95%) to give the title compound (91 mg, 97% purity, 56% yield) as yellow solids.

LC-MS (ESI): R_{T}=2.34 min, mass calcd. for C₃₄H₄₆FN₅O₅S 655.3, m/z found 656.8 [M+H]⁺.

### Intermediates I-10-17A (a Single Stereoisomer) and I-10-17B (a Single Stereoisomer): (S)-Ethyl 6-4(4aR*,8aS*)-6-(3-(tert-butoxy)-2,2-dimethyl-3-oxopropyl)hexahydro-2H-pyrido[4,3-b][1,4]oxazin-4(3H)-yl)methyl)-4-(3-fluoro-2-methylphenyl)-2-(thiazol-2-yl)-1,4-dihydropyrimidine-5-carboxylate (a Single Stereoisomer) and (S)-ethyl 6-(((4aS*,8aR*)-6-(3-(tert-butoxy)-2,2-dimethyl-3-oxopropyl)hexahydro-2H-pyrido [4,3-b][1,4]oxazin-4(3H)-yl)methyl)-4-(3-fluoro-2-methylphenyl)-2-(thiazol-2-yl)-1,4-dihydropyrimidine-5-carboxylate (a Single Stereoisomer)

A racemic mixture of (cis)-(S)-ethyl 6-((6-(3-(ter t-butoxy)-2,2-dimethyl-3-oxopropyl)hexahydro-2H-pyrido[4,3-b][1,4]oxazin-4 (31-1)-yl)methyl)-4-(3-fluoro-2-methylphenyl)-2-(thiazol-2-yl)-1,4-dihydropyrimidine-5-carboxylate I-10-17 (135 mg, 92% purity, 0.189 mmol) was separated by chiral Prep. HPLC (separation condition: Column: Chiralpak IB 5 um 20*250 mm; Mobile Phase: Hex:EtOH=95:5 at 18 mL/min; Temp: 30° C.; Wavelength: 214 nm) to afford the title compound I-10-17A (68 mg, 90% purity from ¹H NMR, 49% yield, 99.9% stereopure) and I-10-17B (64 mg, 90% purity from ¹H NMR, 46% yield, 99.2% stereopure) as yellow solids.

Intermediate I-10-17A: Chiral analysis (Column: Chiralpak IB 5 um 4.6*250 mm; Mobile Phase: Hex:EtOH=95:5 at 1.0 mL/min; Temp: 30° C.; Wavelength: 254 nm, R_{T}=4.818 min). ¹H NMR (400 MHz, CDCl₃) δ 9.71 (s, 1H), 7.81 (d, J=3.2 Hz, 1H), 7.40 (d, J=2.8 Hz, 1H), 7.08-7.02 (m, 1H), 6.98-6.96 (m, 1H), 6.91-6.87 (m, 1H), 6.00 (s, 1H), 4.10-3.95 (m, 5H), 3.87-3.80 (m, 2H), 2.98-2.96 (m, 1H), 2.85-2.63 (m, 3H), 2.59-2.52 (m, 5H), 2.42-2.40 (m, 3H), 1.78-1.74 (m, 2H), 1.42 (s, 9H), 1.14-1.08 (m, 9H).

Intermediate I-10-17B: Chiral analysis (Column: Chiralpak IB 5 um 4.6*250 mm; Mobile Phase: Hex:EtOH=95:5 at 1.0 mL/min; Temp: 30° C.; Wavelength: 254 nm, R_{T}=6.445 min). ¹H NMR (400 MHz, CDCl₃) δ 9.74 (s, 1H), 7.81 (d, J=2.8 Hz, 1H), 7.40 (d, J=3.2 Hz, 1H), 7.11-7.06 (m, 1H), 7.01-6.99 (m, 1H), 6.92-6.88 (m, 1H), 6.02 (s, 1H), 4.31 (d, J=17.2 Hz, 1H), 4.10-4.01 (m, 2H), 3.92-3.80 (m, 4H), 2.89-2.85 (m, 3H), 2.73-2.71 (m, 1H), 2.65-2.54 (m, 5H), 2.41-2.38 (m, 2H), 2.26-2.23 (m, 1H), 1.80 (br s, 2H), 1.45 (s, 9H), 1.14-1.09 (m, 9H).

### Compound I-10A: 3-((4aR*,8aS*)-4-0(S)-5-(Ethoxycarbonyl)-6-(3-fluoro-2-methylphenyl)-2-(thiazol-2-yl)-3,6-dihydropyrimidin-4-yl)methyl)hexahydro-2H-pyrido[4,3-b][1,4]oxazin-6(7H)-yl)-2,2-dimethylpropanoic acid (a Single Stereoisomer)

To a solution of (S)-ethyl 6-4(4aR*,8aS*)-6-(3-(tert-butoxy)-2,2-dimethyl-3-oxopropyl)hexahydro-2H-pyrido[4,3-b][1,4]oxazin-4(3H)-yl)methyl)-4-(3-fluoro-2-methylphenyl)-2-(thiazol-2-yl)-1,4-dihydropyrimidine-5-carboxylate I-10-17A (68 mg, 90% purity, 0.093 mmol) in dichloromethane (2 mL) was added trifluoroacetic acid (2 mL). After stirred at room temperature for 3 hours, the mixture was concentrated under reduced pressure to give a residue, which was purified by C18 column (acetonitrile:water (+0.02% ammonium bicarbonate)=20% to 70%) to give the title compound (45 mg, 99.6% purity, 80% yield) as yellow solids.

LC-MS (ESI): R_{T}=3.839 min, mass calcd. for C₃₀H₃₈FN₅O₅S 599.3, m/z found 600.3 [M+H]⁺. ¹H NMR (400 MHz, CD₃OD) δ 7.94 (d, J=3.2 Hz, 1H), 7.74 (d, J=3.2 Hz, 1H), 7.17-7.08 (m, 2H), 6.96-6.92 (m, 1H), 5.98 (s, 1H), 4.37 (d, J=16.8 Hz, 1H), 4.13-3.86 (m, 6H), 3.56-3.50 (m, 1H), 3.32-3.25 (m, 1H), 3.18-3.00 (m, 6H), 2.60-2.57 (m, 1H), 2.53 (d, J=2.0 Hz, 3H), 2.04-2.02 (m, 2H), 1.26 (s, 3H), 1.24 (s, 3H), 1.14 (t, J=7.2 Hz, 3H).

### Compound I-11A: (S)-3-((4aR*,8aS*)-1-((5-(Ethoxycarbonyl)-6-(3-fluoro-2-methylphenyl)-2-(thiazol-2-yl)-3,6-dihydropyrimidin-4-yl)methyl)hexahydro-1H-pyrido[3,4-b][1,4]oxazin-6(7H)-yl)-2,2-dimethylpropanoic acid (a Single Stereoisomer)

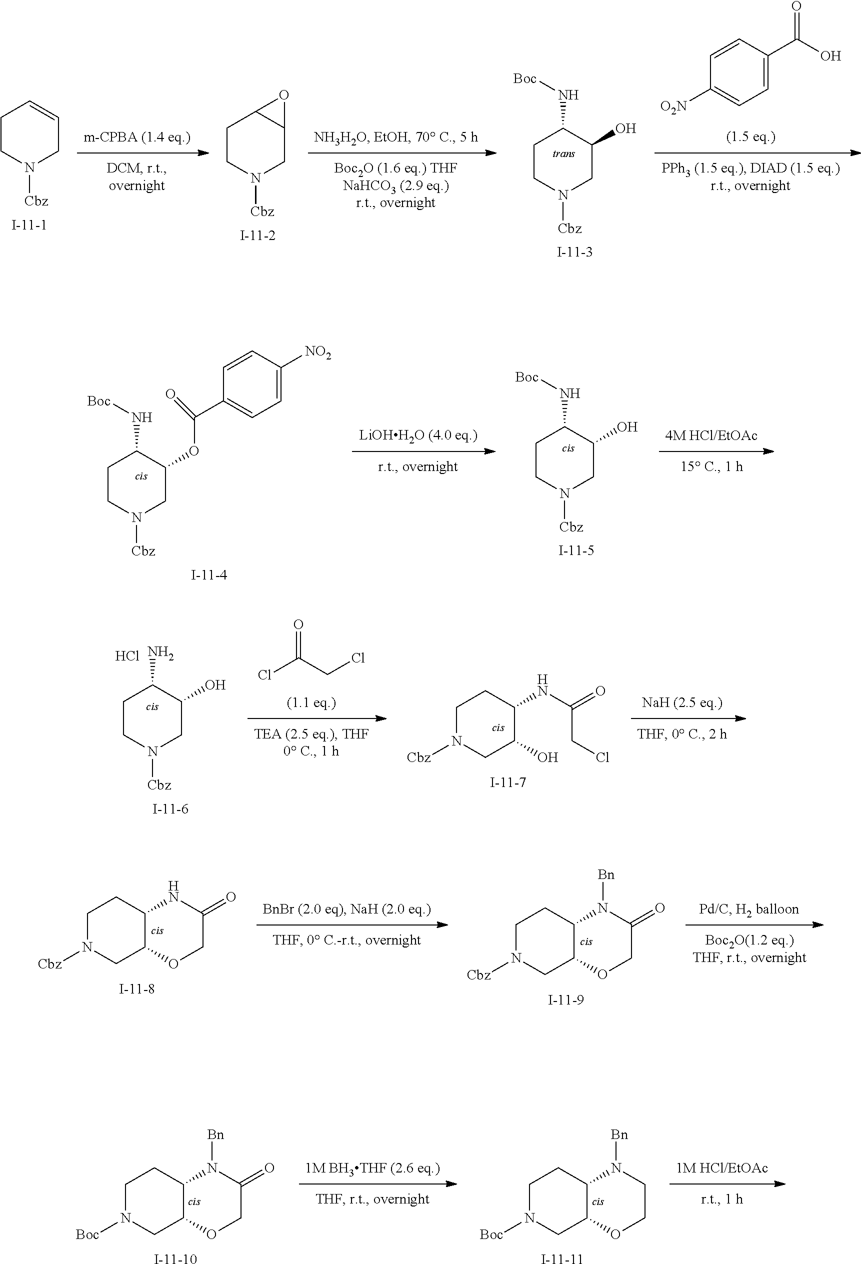

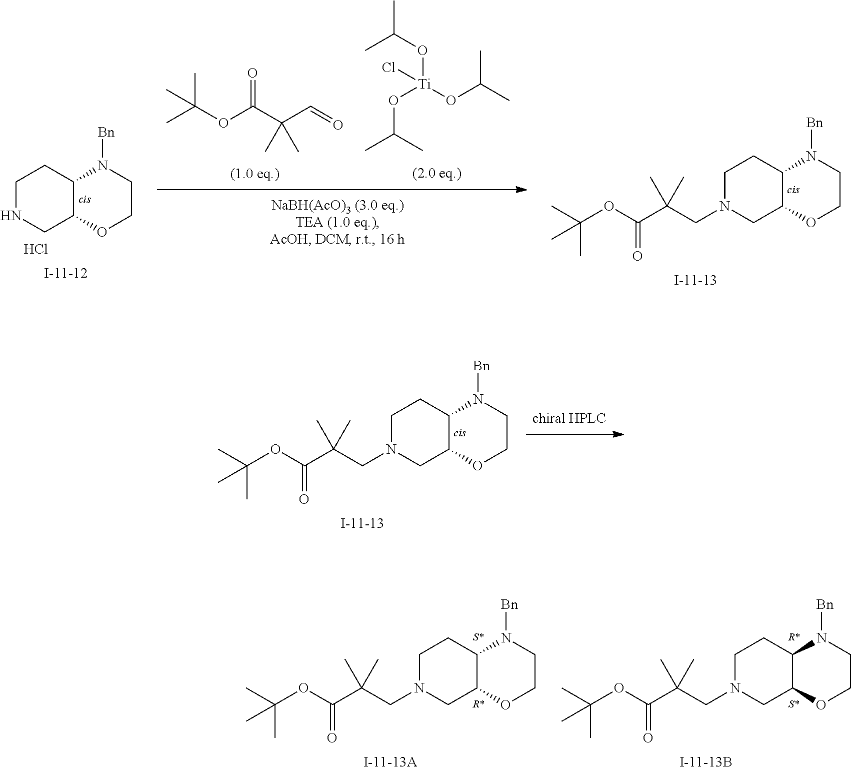

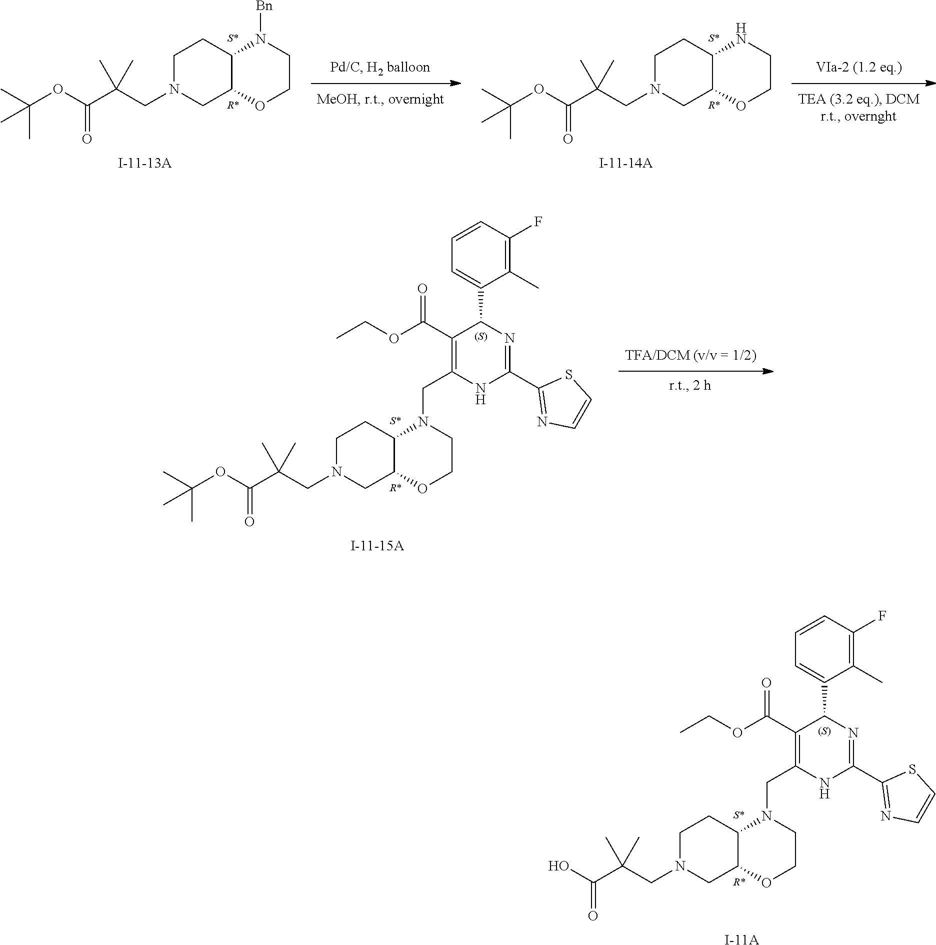

### Intermediate I-11-2: Benzyl 7-oxa-3-azabicyclo[4.1.0]heptane-3-carboxylate

The solution of benzyl 5,6-dihydropyridine-1(2H)-carboxylate (25.0 g, 115 mmol) in dichloromethane (400 mL) was cooled to 0° C. A solution of m-chloroperoxybenzoic acid (27.8 g, 160 mmol) in dichloromethane (100 mL) was added dropwise and the resulting colorless reaction mixture was warmed to room temperature. After stirred at room temperature overnight, the reaction mixture was washed with 5% sodium sulfite aqueous solution (300 mL) three times and brine (500 mL). The organic layer was dried over Na₂SO₄(s), filtered and concentrated in vacuo to give the title compound (26.7 g, 84% purity, 84% yield) as yellow oil.

LC-MS (ESI): R_{T}=1.26 min, mass calcd. for C₁₃H₁₅NO₃ 233.1, m/z found 251.2 [M+18]⁺.

### Intermediates I-11-3 and I-10-4: (trans)-Benzyl 4-((tert-butoxycarbonyl)amino)-3-hydroxypiperidine-1-carboxylate (a Mixture of 2 Stereoisomers) and (trans)-benzyl 3-((tert-butoxycarbonyl)amino)-4-hydroxypiperidine-1-carboxylate (a Mixture of 2 Stereoisomers)

The solution of benzyl 7-oxa-3-azabicyclo[4.1.0]heptane-3-carboxylate I-11-2 (26.7 g, 84% purity, 96.3 mmol) in 25-30% ammonium hydroxide aqueous solution (300 mL) and ethanol (250 mL) was stirred at 70° C. for 5 hours. The reaction mixture was concentrated in vacuo by half. The resultant solution was diluted with tetrahydrofuran (300 mL), then sodium bicarbonate (24.0 g, 287 mmol) and di-tert-butyl dicarbonate (34.0 g, 150 mmol) added dropwise. The reaction was allowed to stirred at room temperature overnight. Then it was quenched with saturated ammonium chloride aqueous (200 mL) and the layers separated. The aqueous phase was further extracted with ethyl acetate (150 mL) twice. The combined organic layers were concentrated under reduced pressure to give a residue, which was purified by silica gel column chromatography (petroleum ether:ethyl acetate=10:1 to 4:1) to give the title compounds I-11-3 (17.0 g, 90% purity from ¹H NMR, 42% yield) and I-10-4 (9.0 g, 90% purity from ¹H NMR, 23% yield) as white solids.

Intermediate I-11-3: LC-MS (ESI): R_{T}=1.54 min, mass calcd. for C18H₂₆N₂O₅ 350.2, m/z found 351.5 [M+H]⁺. ¹H NMR (400 MHz, CDCl₃) δ 7.38-7.33 (m, 5H), 5.16 (s, 2H), 4.76-4.75 (m, 1H), 4.38-4.08 (m, 2H), 3.49-3.42 (m, 2H), 2.86-2.68 (m, 2H), 2.01-1.97 (m, 1H), 1.86 (s, 1H), 1.49 (s, 9H).

Intermediate I-10-4: ¹H NMR (400 MHz, CDCl₃) δ 7.45-7.35 (m, 5H), 5.23-5.14 (m, 2H), 4.88-4.71 (m, 1H), 4.13-3.92 (m, 2H), 3.69-3.46 (m, 3H), 3.27-3.00 (m, 2H), 2.03-1.75 (m, 2H), 1.49 (s, 9H).

### Intermediate I-11-4: (cis)-Benzyl 4-((tert-butoxycarbonyl)amino)-3-((4-nitrobenzoyl)oxy)piperidine-1-carboxylate (a Mixture of 2 Stereoisomers)

To a solution of (trans)-benzyl 4-((tert-butoxycarbonyl)amino)-3-hydroxypiperidine-1-carboxylate I-11-3 (37.0 g, 95% purity, 101 mmol), triphenylphosphine (39.4 g, 150 mmol) and 4-nitrobenzoic acid (25.0 g, 150 mmol) in tetrahydrofuran (500 mL) was added diisopropyl azodicarboxylate (30.4 g, 150 mmol) dropwise at 0° C. After stirred at room temperature overnight under nitrogen atmosphere, the reaction mixture was poured into water (200 mL) and extracted with ethyl acetate (150 mL) for three times. The combined organic phases were washed with brine (150 mL), dried over Na₂SO₄₍ₛ₎ and filtered. The filtrate was concentrated and purified by silica gel column chromatography (petroleum ether:ethyl acetate=6:1) to give the title compound (23.0 g, 85% purity, 39% yield) as white solids.

LC-MS (ESI): R_{T}=1.75 min, mass calcd. for C₂₅H₂₉N₃O₈ 499.2, m/z found 444.1 [M-56+H]⁺.

### Intermediate I-11-5: (cis)-Benzyl 4-((tert-butoxycarbonyl)amino)-3-hydroxypiperidine-1-carboxylate (a Mixture of 2 Stereoisomers)

To a solution of (cis)-benzyl 4-((tert-butoxycarbonyl)amino)-3-((4-nitrobenzoyl)oxy)piperidine-1-carboxylate I-11-4 (23.0 g, 85% purity, 39.0 mmol) in tetrahydrofuran (150 mL) and water (150 mL) was added lithium hydroxide monohydrate (6.60 g, 157 mmol). After stirred at room temperature overnight, the reaction mixture was diluted with water (150 mL), removed tetrahydrofuran in vacuo and extracted with ethyl acetate (150 mL) for three times. The combined organic layers were washed with brine (150 mL), dried over Na₂SO₄₍ₛ₎ and filtered. The filtrate was concentrated and purified by silica gel column chromatography (petroleum ether:ethyl acetate=3:1) to give the title compound (15.0 g, 90% purity from ¹H NMR, 98% yield) as white solids.

¹H NMR (400 MHz, CDCl₃) δ 7.39-7.31 (m, 5H), 5.17 (s, 2H), 5.03-5.02 (m, 1H), 4.35-4.11 (m, 2H), 3.94 (s, 1H), 3.79-3.66 (m, 1H), 3.14-2.89 (m, 2H), 2.18-1.93 (m, 1H), 1.83-1.69 (m, 2H), 1.48 (s, 9H).

### Intermediate I-11-6: (cis)-Benzyl 4-amino-3-hydroxypiperidine-1-carboxylate hydrochloride (a Mixture of 2 Stereoisomers)

To a solution of (cis)-benzyl 4-((tert-butoxycarbonyl)amino)-3-hydroxypiperidine-1-carboxylate I-11-5 (15.0 g, 90% purity, 38.5 mmol) in 4 M hydrochloride in ethyl acetate (50 mL) was stirred at 15° C. for 1 hour. The reaction mixture was concentrated in vacuo to give the title compound (12.0 g, 90% purity from ¹H NMR, 98% yield) as white solids.

¹H NMR (400 MHz, DMSO-d₆) δ 8.13 (s, 3H), 7.37-7.31 (m, 5H), 5.62 (s, 1H), 5.07 (s, 2H), 3.97 (d, J=12.0 Hz, 2H), 3.88 (s, 1H), 3.25 (d, J=8.0 Hz, 1H), 3.14-2.82 (m, 2H), 1.81-1.69 (m, 2H).

### Intermediate I-11-7: (cis)-Benzyl 4-(2-chloroacetamido)-3-hydroxypiperidine-1-carboxylate (a Mixture of 2 Stereoisomers)

To a solution of benzyl 4-amino-3-hydroxypiperidine-1-carboxylate hydrochloride I-11-6 (11.0 g, 90% purity, 34.5 mmol), triethylamine (8.73 g, 86.3 mmol) in tetrahydrofuran (300 mL) was added 2-chloroacetyl chloride (4.29 g, 38.0 mmol) dropwised at 0° C. After stirred at 0° C. for 1 hour, the reaction mixture was diluted with ethyl acetate (200 mL), washed with brine (200 mL) twice. The separated organic layer was dried over Na₂SO₄(s) and filtered. The filtrate was concentrated and purified by silica gel column chromatography (petroleum ether:ethyl acetate=1:1) to give the title compound (11.0 g, 90% purity from ¹H NMR, 88% yield) as white solids.

¹H NMR (400 MHz, CDCl₃) δ 7.48-7.34 (m, 5H), 7.06 (s, 1H), 5.19 (s, 1H), 4.42-4.23 (m, 2H), 4.09-3.96 (m, 4H), 3.14-2.89 (m, 2H), 1.92-1.80 (m, 2H)

### Intermediate I-11-8: (cis)-Benzyl 2-oxohexahydro-1H-pyrido[3,4-b][1,4]oxazine-6(7H)-carboxylate (a Mixture of 2 Stereoisomers)

A solution of (cis)-benzyl 4-(2-chloroacetamido)-3-hydroxypiperidine-1-carboxylate I-11-7 (4.00 g, 90% purity, 11.0 mmol) in tetrahydrofuran (1300 mL) under nitrogen atmosphere at 0° C. was added 60% wt. sodium hydride in mineral oil (1.10 g, 27.5 mmol). After stirred at room temperature for 2 hours, the reaction mixture was poured into water (200 mL) and extracted with ethyl acetate (200 mL) twice. The combined organic phases were washed with brine (200 mL), dried over Na₂SO₄₍ₛ₎ and filtered. The filtrate was concentrated and purified by C18 column (acetonitrile:water=30% to 95%) to give the title compound (1.50 g, 98% purity from ¹H NMR, 46% yield) as white solids. ¹H NMR (400 MHz, CDCl₃) δ 7.36-7.31 (m, 5H), 7.00 (s, 1H), 5.15 (s, 2H), 4.58-4.37 (m, 1H), 4.31-4.18 (m, 3H), 3.83-3.80 (m, 1H), 3.42-3.38 (m, 1H), 3.15-3.00 (m, 1H), 2.88-2.68 (m, 1H), 2.02-1.92 (m, 1H), 1.83-1.80 (m, 1H).

### Intermediate I-11-9: (cis)-Benzyl 1-benzyl-2-oxohexahydro-1H-pyrido[3,4-b][1,4]oxazine-6(7H)-carboxylate (a Mixture of 2 Stereoisomers)

A solution of (cis)-benzyl 2-oxohexahydro-1H-pyrido[3,4-b][1,4]oxazine-6 (7H)-carboxylate I-11-8 (1.60 g, 98% purity, 5.40 mmol) in tetrahydrofuran (60 mL) under nitrogen atmosphere at 0° C. was added 60% wt. sodium hydride in mineral oil (431 mg, 10.7 mmol). Then benzyl bromide (1.85 g, 10.8 mmol) was added. After stirred at room temperature overnight, the reaction mixture was poured into water (100 mL) and extracted with ethyl acetate (100 mL) twice. The combined organic phases were washed with brine (100 mL), dried over Na₂SO₄₍ₛ₎ and filtered. The filtrate was concentrated and purified by silica gel column chromatography (petroleum ether:ethyl acetate=10:1) to give the title compound (2.00 g, 90% purity from ¹H NMR, 87% yield) as white solids.

¹H NMR (400 MHz, CDCl₃) δ 7.34-7.24 (m, 10H), 5.32-5.28 (m, 1H), 5.14-5.13 (m, 2H), 4.56-4.17 (m, 4H), 3.99 (d, J=15.2 Hz, 1H), 3.77-3.71 (m, 1H), 3.17-3.13 (m, 1H), 2.97-2.88 (m, 1H), 2.74-2.59 (m, 1H), 1.97-1.83 (m, 2H).

### Intermediate I-11-10: (cis)-tert-Butyl 1-benzyl-2-oxohexahydro-1H-pyrido[3,4-b][1,4]oxazine-6(7H)-carboxylate (a Mixture of 2 Stereoisomers)

To a solution of (cis)-benzyl 1-benzyl-2-oxohexahydro-1H-pyrido[3,4-b][1,4]oxazine-6(7H)-carboxylate I-11-9 (2.00 g, 90% purity, 4.73 mmol) in tetrahydrofuran (20 mL) was added di-tert-butyl dicarbonate (1.20 g, 5.50 mmol) and 10% palladium on activated carbon wt. (1.00 g) at room temperature. After stirred at room temperature under hydrogen atmosphere balloon overnight, the reaction mixture was filtered and concentrated to afford the title compound (1.65 g, 90% purity from ¹H NMR, 91% yield) as light yellow oil.

LC-MS (ESI): R_{T}=1.58 min, mass calcd. for C₁₉H₂₆N₂O₄ 346.2, m/z found 291.2 [M+H-56]⁺. ¹H NMR (400 MHz, CDCl₃) δ 7.42-7.29 (m, 5H), 5.30 (d, J=20.0 Hz, 1H), 4.67-4.11 (m, 4H), 4.03 (d, J=20.0 Hz, 1H), 3.80-3.67 (m, 1H), 3.18-3.12 (m, 1H), 2.96-2.80 (m, 1H), 2.68-2.51 (m, 1H), 1.96-1.83 (m, 2H), 1.47 (s, 9H).

### Intermediate I-11-11: (cis)-tert-Butyl 1-benzylhexahydro-1H-pyrido[3,4-b][1,4]oxazine-6(7H)-carboxylate (a Mixture of 2 Stereoisomers)

To a solution of (cis)-tert-butyl 1-benzyl-2-oxohexahydro-1H-pyrido[3,4-b][1,4]oxazine-6(7H)-carboxylate I-11-10 (1.30 g, 90% purity, 3.38 mmol) in tetrahydrofuran (10 mL) was added dropwise 1 M borane-tetrahydrofuran complex in tetrahydrofuran (9 mL, 9 mmol) at 0° C. After stirred at room temperature overnight under nitrogen atmosphere, the reaction mixture was acidified with 1 M hydrochloride aqueous solution to pH 5 and extracted with dichloromethane (20 mL) for three times. The combined organic layers were washed with brine (20 mL), dried over Na₂SO₄₍ₛ₎ and filtered. The filtrate was concentrated to give a residue, which was purified by silica gel column chromatography (petroleum ether:ethyl acetate=10:1 to 2:1) to afford the title compound (950 mg, 90% purity from ¹H NMR, 76% yield) as white solids.

¹H NMR (400 MHz, CDCl₃) δ 7.42-7.29 (m, 5H), 4.26-4.08 (m, 3H), 3.93-3.83 (m, 1H), 3.70-3.51 (m, 4H), 2.91-2.80 (m, 1H), 2.78-2.64 (m, 2H), 2.61-2.49 (m, 1H), 2.34-2.31 (m, 1H), 2.13-2.06 (m, 1H), 1.45 (s, 9H).

### Intermediate I-11-12: (cis)-1-Benzyloctahydro-1H-pyrido[3,4-b][1,4]oxazine hydrochloride (a Mixture of 2 Stereoisomers)

To a solution of (cis)-tert-butyl 1-benzylhexahydro-1H-pyrido[3,4-b][1,4]oxazine-6(7H)-carboxylate I-11-11(950 mg, 90% purity, 2.57 mmol) in dichloromethane (8 mL) was added 4 M hydrochloride in ethyl acetate (6 mL, 24 mmol) under nitrogen atmosphere. After stirred at room temperature under nitrogen atmosphere for 1 hour, the reaction mixture was concentrated to afford the title compound (760 mg, 90% purity from ¹H NMR, 99% yield) as white solids.

LC-MS (ESI): R_{T}=1.28 min, mass calcd. for C₁₄H₂₁ClN₂O 286.1, m/z found 233.3 [M+H−HCl]⁺. ¹H NMR (400 MHz, CD₃OD) δ 7.70 (s, 2H), 7.50 (s, 3H), 4.51-4.38 (m, 3H), 4.21-4.18 (m, 1H), 4.10-4.00 (m, 1H), 3.92-3.89 (m, 1H), 3.63-3.48 (m, 3H), 3.38-3.34 (m, 1H), 3.21-3.11 (m, 2H), 2.66-2.53 (m, 1H), 2.39-2.27 (m, 1H).

### Intermediate I-11-13: (cis)-tert-Butyl 3-(1-benzylhexahydro-1H-pyrido[3,4-b][1,4]oxazin-6(7H)-yl)-2,2-dimethylpropanoate (a Mixture of 2 Stereoisomers)

To a solution of (cis)-1-benzyloctahydro-1H-pyrido[3,4-b][1,4]oxazine hydrochloride I-11-12 (600 mg, 90% purity, 2.01 mmol) in dichloromethane (10 mL) was added triethylamine (200 mg, 1.98 mmol). After stirred at 20° C. for 20 minutes, acetic acid (1.5 mL), tert-butyl 2,2-dimethyl-3-oxopropanoate (495 mg, 70% purity, 2.01 mmol) and 1 M triisopropoxytitanium(IV) chloride in hexanes (4 mL, 4 mmol) were added. The mixture was stirred at 20° C. for 40 minutes, then sodium triacetoxyborohydride (1.30 g, 6.13 mmol) was added. After stirred at 20° C. for 16 hours, the reaction mixture was quenched with saturated sodium bicarbonate aqueous solution (30 mL). The mixture was extracted with dichloromethane (20 mL) for three times. The combined organic phases were washed with brine (10 mL), dried over Na₂SO₄₍ₛ₎ and filtered. The filtrate was concentrated to give a residue, which was purified by C18 column (acetonitrile:water=40% to 70%) to give the title compound (800 mg, 90% purity from ¹H NMR, 92% yield) as light yellow oil.

LC-MS (ESI): R_{T}=2.09 min, mass calcd. for C₂₃H₃₆N₂O₃ 388.3, m/z found 389.4 [M+H]⁺. ¹H NMR (400 MHz, CDCl₃) δ 7.34-7.22 (m, 5H), 3.89-3.85 (m, 1H), 3.67-3.52 (m, 4H), 2.98 (d, J=12.4 Hz, 1H), 2.88-2.79 (m, 1H), 2.70-2.63 (m, 1H), 2.58-2.56 (m, 1H), 2.50 (d, J=13.6 Hz, 1H), 2.42 (d, J=13.6 Hz, 1H), 2.30 (t, J=12.4 Hz, 2H), 2.16-2.04 (m, 2H), 1.48-1.36 (m, 1H), 1.44 (s, 9H), 1.12 (s, 6H).

### Intermediates I-11-13A (a Single Stereoisomer) and I-11-13B (a Single Stereoisomer): tert-Butyl 3-((4aR*,8aS*)-1-benzylhexahydro-1H-pyrido[3,4-b][1,4]oxazin-6(7H)-yl)-2,2-dimethylpropanoate (a Single Stereoisomer) and tert-butyl 3-((4aS*, 8aR*)-1-benzylhexahydro-1H-pyrido[3,4-b][1,4]oxazin-6(7H)-yl)-2,2-dimethylpropanoate (a Single Stereoisomer)

A racemic mixture of (cis)-tert-butyl 3-(1-benzylhexahydro-1H-pyrido[3,4-b][1,4]oxazin-6(7H)-yl)-2,2-dimethylpropanoate I-11-13 (800 mg, 90% purity, 1.85 mmol) was separated by chiral Prep. HPLC (separation conditon: Column: Chiralpak IC 5 μm 20*250 mm; Mobile Phase: Hex:EtOH=98:2 at 25 mL/min; Temp: 30° C.; Wavelength: 230 nm) to give the title compounds I-11-13A (320 mg, 90% purity from ¹H NMR, 40% yield, 100% stereopure) and I-11-13B (330 mg, 90% purity from ¹H NMR, 41% yield, 99.6% stereopure) as yellow solids.

Intermediate I-11-13A: LC-MS (ESI): R_{T}=2.09 min, mass calcd. for C₂₃H₃₆N₂O₃ 388.3, m/z found 389.5 [M+F1]⁺. Chiral analysis (Column: Chiralpak IC 5 μm 4.6*250 mm; Mobile Phase: Hex:EtOH=98:2 at 1 mL/min; Temp: 30° C.; Wavelength: 214 nm, R_{T}=4.843 min). ¹H NMR (400 MHz, CDCl₃) δ 7.35-7.22 (m, 5H), 3.90-3.86 (m, 1H), 3.69-3.51 (m, 4H), 2.98 (d, J=12.4 Hz, 1H), 2.86-2.80 (m, 1H), 2.70-2.63 (m, 1H), 2.61-2.55 (m, 1H), 2.50 (d, J=13.6 Hz, 1H), 2.42 (d, J=13.6 Hz, 1H), 2.33-2.27 (m, 2H), 2.17-2.08 (m, 2H), 1.49-1.36 (m, 1H), 1.44 (s, 9H), 1.12 (s, 6H).

Intermediate I-11-13B: LC-MS (ESI): R_{T}=2.09 min, mass calcd. for C₂₃H₃₆N₂O₃ 388.3, m/z found 389.5 [M+H]⁺. Chiral analysis (Column: Chiralpak IC 5 μm 4.6*250 mm; Mobile Phase: Hex:EtOH=98:2 at 1 mL/min; Temp: 30° C.; Wavelength: 214 nm, R_{T}=6.048 min). ¹H NMR (400 MHz, CDCl₃) δ 7.34-7.22 (m, 5H), 3.91-3.86 (m, 1H), 3.67-3.53 (m, 4H), 2.98 (d, J=12 Hz, 1H), 2.90-2.79 (m, 1H), 2.70-2.64 (m, 1H), 2.60-2.55 (m, 1H), 2.49 (d, J=13.2 Hz, 1H), 2.42 (d, J=13.2 Hz, 1H), 2.33-2.26 (m, 2H), 2.17-2.05 (m, 2H), 1.50-1.38 (m, 1H), 1.44 (s, 9H), 1.12 (s, 6H).

### Intermediate I-11-14A: tert-Butyl 3-((4aR*,8aS*)-hexahydro-1H-pyrido[3,4-b][1,4]oxazin-6(7H)-yl)-2,2-dimethylpropanoate (a Single Stereoisomer)

To a solution of tert-butyl 3-((4aR*,8aS*)-1-benzylhexahydro-1H-pyrido[3,4-b][1,4]oxazin-6(7H)-yl)-2,2-dimethylpropanoate I-11-13A (150 mg, 90% purity, 0.347 mmol) in methanol (4 mL) was added 10% palladium on activated carbon wt. (60 mg) at room temperature. After stirred at room temperature under hydrogne atmosphere balloon overnight, the reaction mixture was filtered and concentrated to afford the title compound (111 mg, 90% purity from ¹H NMR, 96% yield) as light yellow oil.

¹H NMR (400 MHz, CDCl₃) δ 3.91-3.82 (m, 1H), 3.67-3.62 (m, 1H), 3.56-3.49 (m, 1H), 3.12-3.04 (m, 1H), 3.00-2.96 (m, 1H), 2.87-2.80 (m, 1H), 2.71-2.68 (m, 2H), 2.51-2.46 (m, 2H), 2.44-2.40 (m, 1H), 2.34-2.23 (m, 1H), 2.15-2.01 (m, 1H), 1.52-1.38 (m, 1H), 1.44 (s, 9H), 1.11 (s, 6H).

### Intermediate I-11-15A: (S)-Ethyl 6-4(4aR*,8aS*)-6-(3-(tert-butoxy)-2,2-dimethyl-3-oxopropyl)octahydro-1H-pyrido[3,4-b][1,4]oxazin-1-yl)methyl)-4-(3-fluoro-2-methylphenyl)-2-(thiazol-2-yl)-1,4-dihydropyrimidine-5-carboxylate (a Single Stereoisomer)

To a solution of (S)-ethyl 6-(bromomethyl)-4-(3-fluoro-2-methylphenyl)-2-(thiazol-2-yl)-1,4-dihydropyrimidine-5-carboxylate VIa-2 (170 mg, 95% purity, 0.368 mmol) in dichloromethane (6 mL) was added tert-butyl 3-((4aR*,8aS*)-hexahydro-1H-pyrido[3,4-b][1,4]oxazin-6(7H)-yl)-2,2-dimethylpropanoate I-11-14A (111 mg, 90% purity, 0.335 mmol) and triethylamine (110 mg, 1.09 mmol) at room temperature. After stirring at room temperature overnight, the reaction mixture was cooled to room temperature, diluted with water (10 mL) and extracted with ethyl acetate (10 mL) twice. The combined organic layers were washed with brine (10 mL), dried over Na₂SO₄₍ₛ₎, filtered and concentrated.

The residue was purified by C18 column (acetonitrile:water=50% to 80%) to give the title compound (180 mg, 90% purity from ¹H NMR, 67% yield) as yellow solids. LC-MS (ESI): R_{T}=2.32 min, mass calcd. for C₃₄H₄₆FN₅O₅S 655.3, m/z found 656.5 [M+H]⁺. ¹H NMR (400 MHz, CDCl₃) δ 9.77 (s, 1H), 7.81 (d, J=3.2 Hz, 1H), 7.41 (d, J=3.2 Hz, 1H), 7.08-7.03 (m, 1H), 6.97-6.95 (m, 1H), 6.92-6.87 (m, 1H), 6.00 (s, 1H), 4.10-3.98 (m, 5H), 3.79-3.72 (m, 2H), 3.06-3.02 (m, 1H), 2.98-2.84 (m, 2H), 2.61-2.58 (m, 1H), 2.55 (s, 3H), 2.51-2.43 (m, 4H), 2.33-2.10 (m, 2H), 1.52-1.47 (m, 1H), 1.44 (s, 9H), 1.14-1.10 (m, 9H).

### Compound I-11A: (S)-3-((4aR*,8aS*)-1-((5-(Ethoxycarbonyl)-6-(3-fluoro-2-methylphenyl)-2-(thiazol-2-yl)-3,6-dihydropyrimidin-4-yl)methyphexahydro-1H-pyrido[3,4-b][1,4]oxazin-6(7H)-yl)-2,2-dimethylpropanoic acid (a Single Stereoisomer)

To a solution of (S)-ethyl 6-4(4aR*,8aS*)-6-(3-(tert-butoxy)-2,2-dimethyl-3-oxopropyl)octahydro-1H-pyrido[3,4-b][1,4]oxazin-1-yl)methyl)-4-(3-fluoro-2-methylphenyl)-2-(thiazol-2-yl)-1,4-dihydropyrimidine-5-carboxylate I-11-15A (180 mg, 90% purity, 0.247 mmol) in dichloromethane (8 mL) was added trifluoroacetic acid (4 mL) at room temperature. After stirred at room temperature under nitrogen atmosphere for 2 hours, the reaction mixture was diluted with water (20 mL) and extracted with ethyl acetate (20 mL) twice. The combined organic layers were washed with brine (20 mL), dried over Na₂SO₄₍ₛ₎, filtered and concentrated. The residue was purified by C18 column (acetonitrile:water=60% to 80%) to give the desired compound (90 mg, 97.9% purity, 59% yield) as yellow solids.

LC-MS (ESI): R_{T}=3.835 min, mass calcd. for C₃₀H₃₈FN₅O₅S 599.3, m/z found 600.3.

¹H NMR (400 MHz, CD₃OD) δ 7.93 (d, J=3.2 Hz, 1H), 7.75 (d, J=3.2 Hz, 1H), 7.17-7.08 (m, 2H), 6.97-6.92 (m, 1H), 5.97 (s, 1H), 4.30 (d, J=16.8 Hz, 1H), 4.11-4.03 (m, 5H), 3.93-3.87 (m, 1H), 3.61-3.51 (m, 2H), 3.22 (d, J=14.8 Hz, 1H), 3.11-2.96 (m, 5H), 2.61-2.58 (m, 1.7H), 2.53 (s, 3H), 2.52-2.49 (s, 0.3H), 1.92-1.85 (m, 1H), 1.28 (s, 3H), 1.23 (s, 3H), 1.15 (t, J=7.2 Hz, 3H).

### Compound I-12A: 3-((4aR*,7aS*)-4-4(S)-5-(ethoxycarbonyl)-6-(3-fluoro-2-methylphenyl)-2-(thiazol-2-yl)-3,6-dihydropyrimidin-4-yl)methyl)hexahydropyrrolo[3,4-b][1,4]oxazin-6(2H)-yl)-2,2-dimethyl-3-oxopropanoic acid (a Single Stereoisomer)

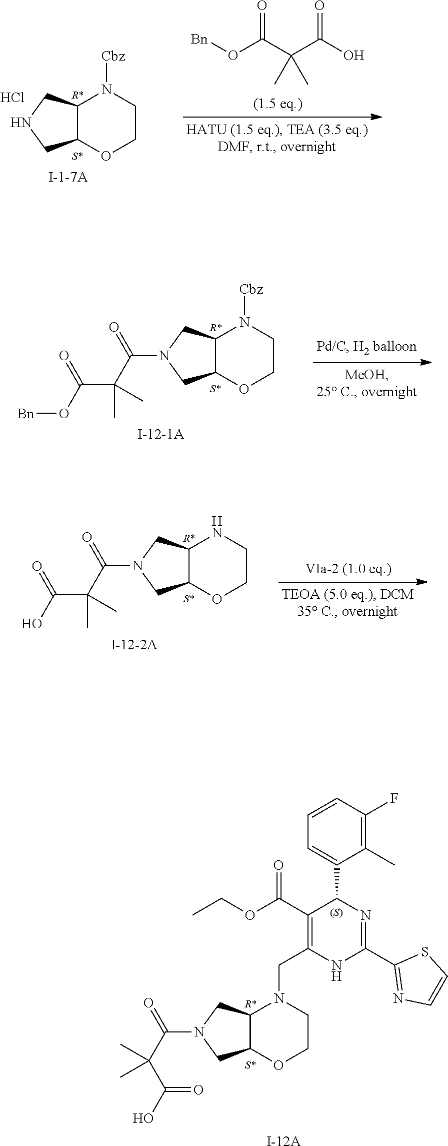

### Intermediate I-12-1A: (4aR*,7aS*)-Benzyl 6-(3-(benzyloxy)-2,2-dimethyl-3-oxopropanoyl)hexahydropyrrolo[3,4-b][1,4]oxazine-4(4aH)-carboxylate (a Single Stereoisomer)

To a solution of 3-(benzyloxy)-2,2-dimethyl-3-oxopropanoic acid (112 mg, 90% purity, 0.454 mmol) in N,N-dimethylformamide (2 mL) was added 2-(3H-[1,2,3]triazolo[4,5-b]pyridin-3-yl)-1,1,3,3-tetramethyl uronium hexafluorophosphate(V) (172 mg, 0.452 mmol) and triethylamine (107 mg, 1.06 mmol) at room temperature under nitrogen atmosphere. After stirred at room temperature for 15 minutes, (4aR*,7aS*)-benzyl hexahydropyrrolo[3,4-b][1,4]oxazine-4(4aH)-carboxylate hydrochloride I-1-7A (100 mg, 90% purity, 0.301 mmol) was added. After stirred at room temperature overnight, the mixture was diluted with water (5 mL) and extracted with ethyl acetate (5 mL) for three times. The combined organic layers were washed with brine (10 mL), dried over

Na₂SO₄(s) and filtered. The filtrate was concentrated and purified by silica gel column chromatography (petroleum ether:ethyl acetate=20:1 to 3:1) to give the title compound (110 mg, 90% purity from ¹H NMR, 70% yield) as yellow oil.

¹H NMR (400 MHz, CDCl₃) δ 7.37-7.30 (m, 10H), 5.30-5.24 (m, 2H), 5.17-5.10 (m, 2H), 4.40 (s, 0.5H), 4.22 (s, 0.5H), 3.89-3.43 (m, 7H), 3.11-2.86 (m, 2H), 1.45-1.35 (m, 6H).

### Intermediate I-12-2A: 3-((4aR*,7aS*)-Hexahydropyrrolo[3,4-b][1,4]oxazin-6(2H)-yl)-2,2-dimethyl-3-oxopropanoic acid (a Single Stereoisomer)

To a solution of (4aR*,7aS*)-benzyl 6-(3-(benzyloxy)-2,2-dimethyl-3-oxopropanoyl)hexahydropyrrolo[3,4-b][1,4]oxazine-4(4aH)-carboxylate I-12-1A (100 mg, 90% purity, 0.179 mmol) in methanol (20 mL) was added 10% palladium on charcoal wt. (200 mg, 0.188 mmol) at room temperature. After stirred at 25° C. under hydrogen atmosphere (balloon) overnight, the mixture was filtered and the filtrate was concentrated to give the title compound (48 mg, 90% purity from ¹H NMR, 99% yield) as yellow oil.

¹H NMR (400 MHz, CDCl₃) δ 4.01 (s, 1H), 3.87-3.76 (m, 1H), 3.67-3.43 (m, 6H), 3.12-2.99 (m, 1H), 2.70-2.63 (m, 1H), 1.38 (s, 3H), 1.35 (s, 3H).

### Compound I-12A: 3-((4aR*,7aS*)-4-0(S)-5-(ethoxycarbonyl)-6-(3-fluoro-2-methylphenyl)-2-(thiazol-2-yl)-3,6-dihydropyrimidin-4-yl)methyl)hexahydropyrrolo[3,4-b][1,4]oxazin-6(2H)-yl)-2,2-dimethyl-3-oxopropanoic acid (a Single Stereoisomer)

To a solution of 3-((4aR*,7aS*)-hexahydropyrrolo[3,4-b][1,4]oxazin-6(2H)-yl)-2,2-dimethyl-3-oxopropanoic acid I-12-2A (48 mg, 90% purity, 0.178 mmol) in dichloromethane (10 mL) was added triethanolamine (133 mg, 0.891 mmol) and (S)-ethyl 6-(bromomethyl)-4-(3-fluoro-2-methylphenyl)-2-(thiazol-2-yl)-1,4-dihydropyrimidine-5-carboxylate VIa-2 (80 mg, 98% purity, 0.179 mmol) at room temperature under nitrogen atmosphere. After stirred at 35° C. overnight, the mixture was diluted with water (20 mL) and extracted with ethyl acetate (20 mL) for three times. The combined organic layers were washed with brine (50 mL), dried over Na₂SO₄₍ₛ₎ and filtered. The filtrate was concentrated and purified by C18 column (acetonitrile:water (+0.1% ammonium bicarbonate)=30% to 95%) to give the title compound (50 mg, 99.5% purity, 46% yield) as yellow solids.

LC-MS (ESI): R_{T}=3.408 min, mass calcd. for C₂₉H₃₄FN₅O₆S 649.3, m/z found 600.2 [M+H]⁺. ¹H NMR (400 MHz, DMSO-d₆) δ 9.49 (br s, 1H), 8.00 (d, J=3.2 Hz, 1H), 7.92 (d, J=3.2 Hz, 1H), 7.20-7.15 (m, 1H), 7.04-7.00 (m, 2H), 5.88 (s, 0.96H), 5.76 (s, 0.04H), 4.14-3.97 (m, 5H), 3.88-3.85 (m, 1H), 3.58-3.54 (m, 2H), 3.47-3.35 (m, 4H), 2.84-2.78 (m, 1H), 2.59-2.54 (m, 1H), 2.44 (s, 3H), 1.26-1.19 (m, 6H), 1.07 (t, J=7.8 Hz, 3H).

### Compound I-13A: 3-((4aR*,7aS*)-4-0(R*)-6-(2-Chloro-3-fluorophenyl)-5-(ethoxycarbonyl)-2-(thiazol-2-yl)-3,6-dihydropyrimidin-4-yl)methyl)hexahydropyrrolo[3,4-b][1,4]oxazin-6(2H)-yl)-2,2-dimethylpropanoic acid (a Single Stereoisomer)

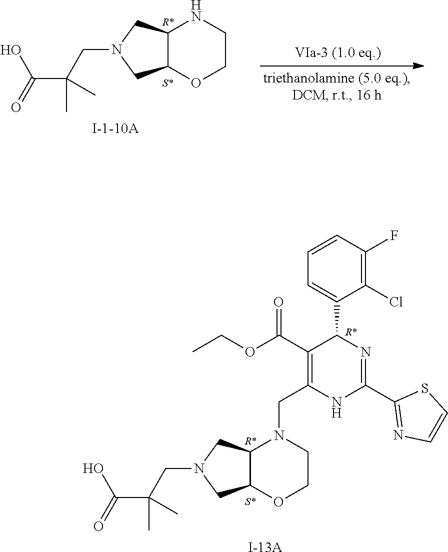

By utilizing the analogous procedure of Method C, the title compound was synthesized from I-1-10A and VIa-3 as yellow solids.

LC-MS (ESI): R_{T}=3.575 min, mass calcd. for C₂₈H₃₃ClFN₅O₅S 605.2, m/z found 606.3 [M+H]⁺. ¹H NMR (400 MHz, DMSO-d₆) δ 13.06 (s, 1H), 9.65 (s, 1H), 8.02 (d, J=3.2 Hz, 1H), 7.95 (d, J=3.2 Hz, 1H), 7.38-7.28 (m, 2H), 7.24-7.22 (m, 1H), 6.01 (s, 1H), 4.07-3.92 (m, 4H), 3.86-3.83 (m, 1H), 3.59-3.54 (m, 1H), 3.29-3.26 (m, 1H), 3.15-3.06 (m, 2H), 2.81-2.67 (m, 5H), 2.57-2.54 (m, 1H), 1.05-1.02 (m, 9H).

### Compound I-14A: 3-((4aR*,7aS*)-4-0(S*)-6-(3-fluoro-2-methylphenyl)-5-(methoxycarbonyl)-2-(thiazol-2-yl)-3,6-dihydropyrimidin-4-yl)methyl)hexahydropyrrolo[3,4-b][1,4]oxazin-6(2H)-yl)-2,2-dimethylpropanoic acid (a Single Stereoisomer)

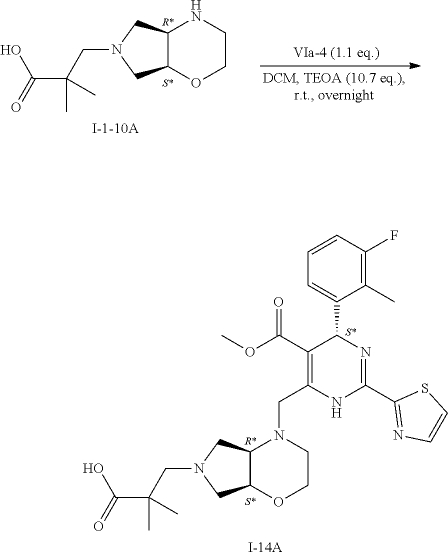

By utilizing the analogous procedure of Method C, the title compound was synthesized from I-1-10A and VIa-4 as yellow solids.

LC-MS (ESI): R_{T}=3.449 min, mass calcd. for C₂₈H₃₄FN₅O₅S 571.3, found 572.3 [M+H]⁺. ¹H NMR (400 MHz, CD₃OD) δ 7.94 (d, J=2.8 Hz, 1H), 7.75 (d, J=3.2 Hz, 1H), 7.17-7.08 (m, 2H), 6.95 (t, J=9.6 Hz, 1H), 5.98 (s, 1H), 4.35-4.26 (m, 2H), 4.06-4.00 (m, 2H), 3.91-3.76 (m, 3H), 3.62 (s, 3H), 3.46 (br s, 2H), 3.40-3.38 (m, 1H), 3.26 (d, J=12.8 Hz, 1H), 3.16 (d, J=12.8 Hz, 1H), 3.05 (td, J=12.4, 2.8 Hz, 1H), 2.69 (d, J=12.0 Hz, 1H), 2.53 (s, 3H), 1.30 (s, 3H), 1.22 (s, 3H).

### Compound I-15A: 3-((4aR*,7aS*)-4-((R*)-6-(2-Chloro-4-fluorophenyl)-5-(methoxycarbonyl)-2-(thiazol-2-yl)-3,6-dihydropyrimidin-4-yl)methyl)hexahydropyrrolo[3,4-b][1,4]oxazin-6(2H)-yl)-2,2-dimethylpropanoic acid (a Single Stereoisomer)

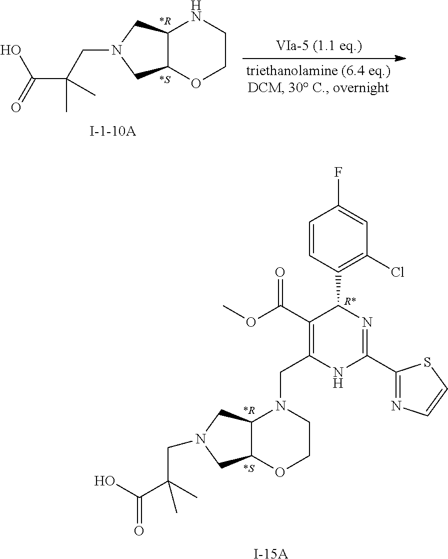 [SMILES: CC(C)(CN1C[C@@H]2OCCN[C@@H]2C1)C(=O)O ; COC(=O)C1=C(CN2CCO[C@H]3CN(CC(C)(C)C(=O)O)C[C@H]32)NC(c2nccs2)=N[C@H]1c1ccc(F)cc1Cl ; "I-15A"]

By utilizing the analogous procedure of Method C, the title compound was synthesized from I-1-10A and VIa-5 as yellow solids.

LC-MS (ESI): R_{T}=3.656 min, mass calcd. for C₂₇H₃₁ClFN₅O₅S 591.2, m/z found 592.1 [M+H]⁺. ¹H NMR (400 MHz, DMSO-d₆) δ 13.07 (s, 1H), 9.69 (s, 1H), 8.02 (d, J=2.8 Hz, 1H), 7.94 (d, J=3.2 Hz, 1H), 7.43-7.36 (m, 2H), 7.19-7.14 (m, 1H), 6.04 (s, 1H), 4.07-3.93 (m, 3H), 3.86-3.83 (m, 1H), 3.60-3.54 (m, 1H), 3.52 (s, 3H), 3.26-3.22 (m, 1H), 3.14-3.05 (m, 2H), 2.81-2.67 (m, 5H), 2.58-2.53 (m, 1H), 1.05 (s, 3H), 1.02 (s, 3H).

### Compound I-16A: 3-((4aR*,7aS*)-4-0(R*)-6-(2-Chloro-3-fluorophenyl)-5-(methoxycarbonyl)-2-(thiazol-2-yl)-3,6-dihydropyrimidin-4-yl)methyl)hexahydropyrrolo[3,4-b][1,4]oxazin-6(2H)-yl)-2,2-dimethylpropanoic acid (a Single Stereoisomer)

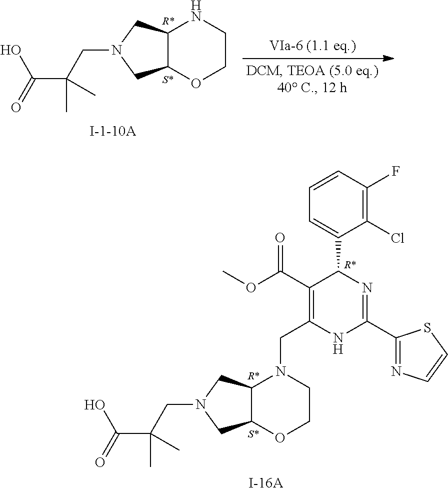 [SMILES: CC(C)(CN1C[C@@H]2OCCN[C@@H]2C1)C(=O)O ; COC(=O)C1=C(CN2CCO[C@H]3CN(CC(C)(C)C(=O)O)C[C@H]32)NC(c2nccs2)=N[C@H]1c1cccc(F)c1Cl ; "I-16A"]

By utilizing the analogous procedure of Method C, the title compound was synthesized from I-1-10A and VIa-6 as yellow solids.

LC-MS (ESI): R_{T}=3.284 min, mass calcd. for C₂₇H₃₁ClFN₅O₅S 591.2 m/z found 592.3 [M+H]⁺. ¹H NMR (400 MHz, DMSO-d₆) δ 13.14 (br s, 1H), 9.72 (s, 1H), 8.02 (d, J=3.2 Hz, 1H), 7.95 (d, J=2.8 Hz, 1H), 7.38-7.29 (m, 2H), 7.22-7.20 (m, 1H), 6.09 (s, 1H), 4.06 (d, J=17.2 Hz, 1H), 3.98-3.94 (m, 2H), 3.86-3.83 (m, 1H), 3.59-3.54 (m, 1H), 3.51 (s, 3H), 3.27-3.22 (m, 1H), 3.17-3.06 (m, 2H), 2.81-2.67 (m, 5H), 2.56-2.51 (m, 1H), 1.05 (s, 3H), 1.02 (s, 3H).

### Compound I-17B: 3-((4aS*,7aS*)-4-(((S*)-6-(3,4-difluoro-2-methylphenyl)-5-(methoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

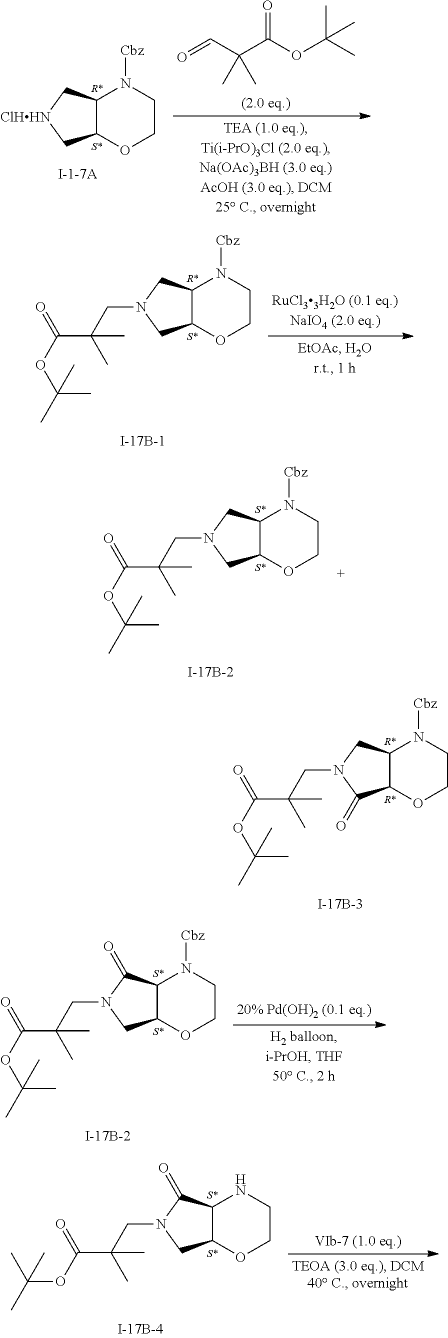

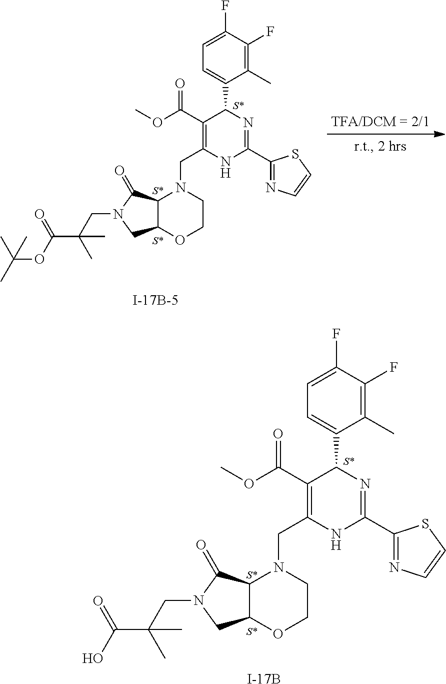 [SMILES: COC(=O)C1=C(CN2CCO[C@H]3CN(CC(C)(C)C(=O)OC(C)(C)C)C(=O)[C@H]32)NC(c2nccs2)=N[C@H]1c1ccc(F)c(F)c1C ; COC(=O)C1=C(CN2CCO[C@H]3CN(CC(C)(C)C(=O)O)C(=O)[C@H]32)NC(c2nccs2)=N[C@H]1c1ccc(F)c(F)c1C ; "I-17B"]

### Intermediates I-17B-1: (4R*, 7S*)-Benzyl 6-(3-(tert-butoxy)-2,2-dimethyl-3-oxopropyl)hexahydropyrrolo[3,4-b][1,4]oxazine-4(4aH)-carboxylate (a Single Stereoisomer)

A suspension of (4R*, 7S*)-benzyl hexahydropyrrolo[3,4-b][1,4]oxazine-4(4aH)-carboxylate hydrochloride I-1-7A (20.3 g, 90% purity, 61.2 mmol) in dichloromethane (200 mL) was added triethylamine (6.19 g, 61.2 mmol). After stirred for 10 minutes, a solution of tert-butyl 2,2-dimethyl-3-oxopropanoate (30.1 g, 70% purity, 122 mmol) and 1 M chlorotriisopropoxytitanium(IV) in dichloromethane (122 mL, 122 mmol) was added at room temperature. After stirred at room temperature for 30 minutes, sodium triacetoxyborohydride (38.9 g, 184 mmol) and acetic acid (11.0 g, 183 mmol) were added into the mixture. After stirred at 25° C. overnight under nitrogen atmosphere, the mixture was quenched with saturated sodium bicarbonate aqueous solution (100 mL) at 0° C. The mixture was filtered through a pad of celite, washed with ethyl acetate (100 mL) twice. The filtrate was concentrated under reduced pressure to give a residue, which was purified by silica gel column chromatography (petroleum ether:ethyl acetate=10:1 to 5:1) to give the title compound (28.0 g, 80% purity from ¹H NMR, 88% yield) as yellow oil. ¹H NMR (400 MHz, CDCl₃) δ 7.38-7.30 (m, 5H), 5.15 (s, 2H), 4.36-4.29 (m, 0.4H), 4.24-4.19 (m, 0.6H), 3.89-3.71 (m, 3H), 3.49-3.32 (m, 2H), 3.21-3.08 (m, 1H), 3.02-2.98 (m, 0.4H), 2.93-2.88 (m, 0.6H), 2.81-2.74 (m, 2H), 2.68-2.57 (m, 2H), 1.43 (s, 3H), 1.39 (s, 6H), 1.10 (s, 4H), 1.09 (s, 2H).

### Intermediates I-17B-2 (a Single Stereoisomer) and I-17B-3 (a Single Stereoisomer): (4S*,7S*)-Benzyl 6-(3-(tert-butoxy)-2,2-dimethyl-3-oxopropyl)-5-oxohexahydropyrrolo[3,4-b][1,4]oxazine-4(4aH)-carboxylate (a Single Stereoisomer) and (4R*, 7R*)-benzyl 6-(3-(tert-butoxy)-2,2-dimethyl-3-oxopropyl)-7-oxohexahydropyrrolo[3,4-b][1,4]oxazine-4(4aH)-carboxylate (a Single Stereoisomer)

To a solution of (4R*, 7S*)-benzyl 6-(3-(tert-butoxy)-2,2-dimethyl-3-oxopropyl)hexahydropyrrolo[3,4-b][1,4]oxazine-4(4 aH)-carboxylate I-17B-1 (56.0 g, 80% purity, 107 mmol) in ethyl acetate (280 mL) and water (280 mL) were added ruthenium(III) chloride trihydrate (2.80 g, 10.7 mmol) and sodium periodate (45.8 g, 214 mmol) at room temperature. After stirred at room temperature for 1 hour, the mixture was quenched with saturated sodium bicarbonate (500 mL) and extracted with ethyl acetate (500 mL) for three times. The combined organic layers were washed with brine (500 mL), dried over Na₂SO₄₍ₛ₎ and filtered. The filtrate was concentrated under reduced pressure to give a residue, which was purified by silica gel column chromatography (petroleum ether:ethyl acetate=5:1 to 1:1) to give the title compound I-17B-2 (10.5 g, 90% purity from ¹H NMR, 20% yield, 99.5% stereopure) as brown oil, the mixture compound of I-17B-2 and I-17B-3 (17.0 g, 90% purity, 33% yield) as brown oil and the title compound I-17B-3 (16.5 g, 90% purity, 32% yield, 90.0% stereopure) as brown solids. Intermediate I-17B-2: Chiral analysis (Column: Chiralpak IG 5 μm 4.6*250 mm; Mobile Phase: Hex:EtOH=50:50 at 1 mL/min; Temp: 30° C.; Wavelength: 214 nm, R_{T}=10.473 min). ¹H NMR (400 MHz, CDCl₃) δ 7.37-7.30 (m, 5H), 5.25-5.15 (m, 2H), 4.81-4.77 (m, 0.5H), 4.62-4.57 (m, 0.5H), 4.13-4.07 (m, 1H), 3.90-3.73 (m, 2H), 3.69-3.64 (m, 1H), 3.59-3.45 (m, 2H), 3.33-3.29 (m, 1H), 3.25 (d, J=11.6 Hz, 1H), 3.08-2.93 (m, 1H), 1.46 (s, 5H), 1.44 (s, 4H), 1.17 (s, 3H), 1.13 (s, 3H).

Intermediate I-17B-3: Chiral analysis (Column: Chiralpak IG 5 um 4.6*250 mm; Mobile Phase: Hex:EtOH=50:50 at 1 mL/min; Temp: 30° C.; Wavelength: 214 nm, R_{T}=15.698 min).

### Intermediates I-17B-2 (a Single Stereoisomer) and I-17B-3 (a Single Stereoisomer): (4S*, 7S*)-Benzyl 6-(3-(tert-butoxy)-2,2-dimethyl-3-oxopropyl)-5-oxohexahydropyrrolo[3,4-b][1,4]oxazine-4(4aH)-carboxylate (a Single Stereoisomer) and (4R*, 7R*)-benzyl 6-(3-(tert-butoxy)-2,2-dimethyl-3-oxopropyl)-7-oxohexahydropyrrolo[3,4-b][1,4]oxazine-4(4aH)-carboxylate (a Single Stereoisomer)

A mixture of I-17B-1 (26.0 g, 90% purity, 54.1 mmol) was separated by chiral Prep. HPLC (Column: Chiralpak IG 5 um 20*250 mm; Mobile Phase: Hex:EtOH=50:50 at 15 mL/min; Temp: 30° C.; Wavelength: 214 nm) to afford the title compounds I-17B-2 (12.7 g, 90% purity from ¹H NMR, 49% yield, 100% stereopure) and I-17B-3 (9.80 g, 90% purity from ¹H NMR, 38% yield, 86.3% stereopure) as yellow solids. Intermediate I-17B-2: LC-MS (ESI): R_{T}=1.423 min, mass calcd. for C₂₃H₃₂N₂O₆ 432.2, m/z found 433.2 [M+H]⁺. Chiral analysis (Column: Chiralpak IG 5 um 4.6*250 mm; Mobile Phase: Hex:EtOH=50:50 at 1 mL/min; Temp: 30° C.; Wavelength: 214 nm, R_{T}=10.491 min). ¹H NMR (300 MHz, CDCl₃) δ 7.41-7.29 (m, 5H), 5.26-5.14 (m, 2H), 4.81-4.77 (m, 0.5H), 4.62-4.57 (m, 0.5H), 4.13-4.07 (m, 1H), 3.90-3.41 (m, 5H), 3.33-3.29 (m, 2H), 3.09-2.92 (m, 1H), 1.45 (s, 5H), 1.43 (s, 4H), 1.17 (s, 3H), 1.13 (s, 3H).

Intermediate I-17B-3: LC-MS (ESI): R_{T}=1.411 min, mass calcd. for C₂₃H₃₂N₂O₆ 432.2, m/z found 433.2 [M+H]⁺. Chiral analysis (Column: Chiralpak IG 5 um 4.6*250 mm; Mobile Phase: Hex:EtOH=50:50 at 1 mL/min; Temp: 30° C.; Wavelength: 214 nm, R_{T}=15.826 min). ¹H NMR (300 MHz, CDCl₃) δ 7.42-7.31 (m, 5H), 5.16 (s, 2H), 4.46-4.36 (m, 1H), 4.01-3.78 (m, 3H), 3.60-3.36 (m, 5H), 3.25-3.11 (m, 1H), 1.42 (s, 9H), 1.15 (s, 6H).

### Intermediate I-17B-4: (4S*, 7 S*)-tert-Butyl 2,2-dimethyl-3-(5-oxohexahydropyrrolo[3,4-b][1,4]oxazin-6(2H)-yl)propanoate (a Single Stereoisomer)

To a solution of (4S*, 7S*)-benzyl 6-(3-(tert-butoxy)-2,2-dimethyl-3-oxopropyl)-5-oxohexahydropyrrolo[3,4-b][1,4]oxazine-4(4aH)-carboxylate I-17B-2 (8.50 g, 90% purity, 17.7 mmol) in propan-2-ol (45 mL) and tetrahydrofuran (45 mL) was added 20% palladium hydroxide on charcoal (1.24 g, 1.77 mmol) at room temperature. After stirred at 50° C. under hydrogen atmosphere balloon for 2 hours, the mixture was filtered through a pad of celite. The filtrate was concentrated under reduced pressure to give the title compound (5.60 g, 90% purity from ¹H NMR, 95% yield) as colorless oil.

¹H NMR (400 MHz, CDCl₃) δ 4.23 (t, J=3.6 Hz, 1H), 3.76-3.72 (m, 1H), 3.66 (d, J=14.0 Hz, 1H), 3.62-3.57 (m, 1H), 3.49-3.45 (m, 1H), 3.41-3.38 (m, 1H), 3.35 (d, J=14.0 Hz, 1H), 3.18 (d, J=11.6 Hz, 1H), 2.88-2.81 (m, 1H), 2.66-2.62 (m, 1H), 1.45 (s, 9H), 1.18 (s, 3H), 1.12 (s, 3H).

### Intermediate I-17B-5: methyl (S*)-6-(((4aS*,7aS*)-6-(3-(tert-butoxy)-2,2-dimethyl-3-oxopropyl)-5-oxohexahydropyrrolo[3,4-b][1,4]oxazin-4(4aH)-yl)methyl)-4-(3,4-difluoro-2-methylphenyl)-2-(thiazol-2-yl)-1,4-dihydropyrimidine-5-carboxylate (a Single Stereoisomer)

By utilizing the analogous procedure of Method C, the title compound was synthesized from I-17B-4 and VIb-7 as yellow solids.

LC-MS(ESI): Rₜ=3.4 min, mass calcd. for C₃₂H₃₉F₂N₅O₆S 659.3, m/z found 660.3 [M+H]⁺.

### Compound I-17B: 3-((4aS*,7aS*)-4-4(S*)-6-(3,4-difluoro-2-methylphenyl)-5-(methoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

To a solution of I-17B-5 (33 mg, 0.05 mmol) in DCM (1.5 mL) in Argonat 0° was added TFA (3.0 mL) and stirred at rt for 2 hrs. The solvent was removed to give a residue, which was extracted with dichloromethane (20 mL) for three times. The combined organic phases were washed with brine (10 mL), dried over Na₂SO₄₍ₛ₎ and filtered. The filtrate was concentrated to give a residue, which was purified by C18 column (acetonitrile:water=40% to 70%) to give the title compound as a yellow solid.

LC-MS(ESI): Rₜ=2.8 min, mass calcd. for C₂₈H₃₁F₂N₅O₆S 603.6, m/z found 604.3 [M+H]⁺. ¹H NMR (400 MHz, METHANOL-d₄) δ: 7.93 (d, 1H), 7.72 (d, 1H), 6.94-7.05 (m, 2H), 5.89 (s, 1H), 4.50-4.68 (m, 2H), 4.27 (m, 1H), 3.74-3.90 (m, 2H), 3.68 (d, 1H), 3.55-3.65 (m, 5H), 3.27-3.40 (m, 2H), 2.83-2.94 (m, 1H), 2.60-2.67 (m, 1H), 2.55 (d, 3H), 1.21 (s, 3H), 1.17 (s, 3H).

### Compound I-18A: 3-((4aS*,7aS*)-4-4(R*)-6-(2-chloro-3-fluorophenyl)-5-(ethoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

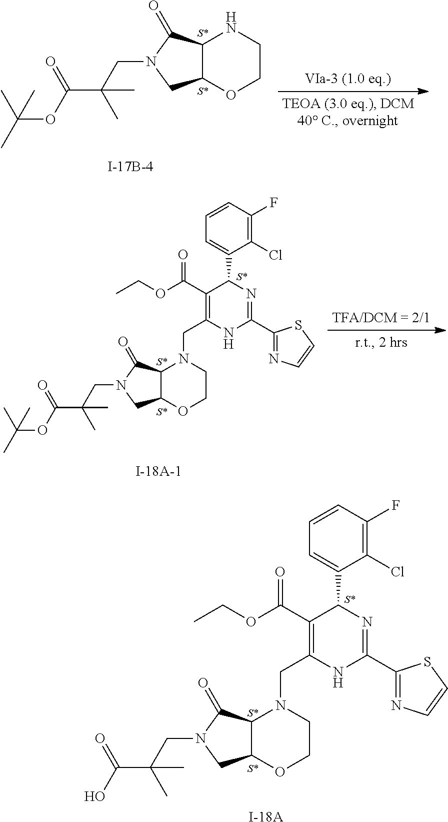 [SMILES: CC(C)(C)OC(=O)C(C)(C)CN1C[C@@H]2OCCN[C@@H]2C1=O ; CCOC(=O)C1=C(CN2CCO[C@H]3CN(CC(C)(C)C(=O)OC(C)(C)C)C(=O)[C@H]32)NC(c2nccs2)=N[C@H]1c1cccc(F)c1Cl ; CCOC(=O)C1=C(CN2CCO[C@H]3CN(CC(C)(C)C(=O)O)C(=O)[C@H]32)NC(c2nccs2)=N[C@H]1c1cccc(F)c1Cl ; "I-18A"]

### Intermediate I-18A-1: ethyl (R*)-6-(((4aS*,7aS*)-6-(3-(tert-butoxy)-2,2-dimethyl-3-oxopropyl)-5-oxohexahydropyrrolo[3,4-b][1,4]oxazin-4(4aH)-yl)methyl)-4-(2-chloro-3-fluorophenyl)-2-(thiazol-2-yl)-1,4-dihydropyrimidine-5-carboxylate (a Single Stereoisomer)

By utilizing the analogous procedure of Method C, the title compound was synthesized from I-17B-4 and VIa-3 as yellow solids.

LC-MS(ESI): Rₜ=3.41 min, mass calcd. for C₃₂H₃₉ClFN₅O₆S 675.2, m/z found 676.3 [M+H]⁺.

### Compound I-18A: 3-((4aS*,7aS*)-4-4(R*)-6-(2-chloro-3-fluorophenyl)-5-(ethoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

By utilizing the analogous procedure of I-17B, the title compound was synthesized from I-18A-1 as yellow solids.

LC-MS(ESI): Rₜ=2.91 min, mass calcd. for C₂₈H₃₁ClFN₅O₆S 619.2, m/z found 620.3 [M+H]⁺. ¹H NMR (400 MHz, CDCl₃) δ: 9.60 (brs, 1H), 7.84 (d, J=3.2 Hz, 1H), 7.44 (d, J=3.2 Hz, 1H), 7.20-7.10 (m, 2H), 7.05-7.01 (m, 1H), 6.26 (s, 1H), 4.65-4.52 (m, 2H), 4.30 (t, J=3.2 Hz, 1H), 4.08-4.03 (q, J=7.2 Hz, 2H), 3.88-3.80 (m, 2H), 3.64-3.60 (d, J=13.9 Hz, 1H), 3.55-3.52 (dd, J=3.6, 11.2 Hz, 1H), 3.45-3.35 (m, 3H), 2.98-2.93 (m, 1H), 2.58 (d, J=11.8 Hz, 1H), 1.26 (s, 3H), 1.23 (s, 3H), 1.15 (t, J=7.1 Hz, 3H).

### Compound I-19A: 3-((4aS*,7aS*)-4-4(S*)-6-(3-fluoro-2-methylphenyl)-5-(methoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

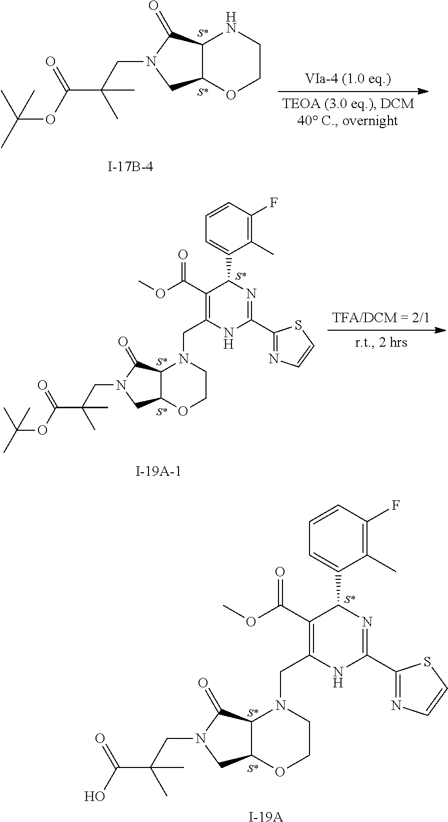 [SMILES: CC(C)(C)OC(=O)C(C)(C)CN1C[C@@H]2OCCN[C@@H]2C1=O ; COC(=O)C1=C(CN2CCO[C@H]3CN(CC(C)(C)C(=O)OC(C)(C)C)C(=O)[C@H]32)NC(c2nccs2)=N[C@H]1c1cccc(F)c1C ; COC(=O)C1=C(CN2CCO[C@H]3CN(CC(C)(C)C(=O)O)C(=O)[C@H]32)NC(c2nccs2)=N[C@H]1c1cccc(F)c1C ; "I-19A"]

### Intermediate I-19A-1: methyl (S*)-6-(((4aS*,7aS*)-6-(3-(tert-butoxy)-2,2-dimethyl-3-oxopropyl)-5-oxohexahydropyrrolo[3,4-b][1,4]oxazin-4(4aH)-yl)methyl)-4-(3-fluoro-2-methylphenyl)-2-(thiazol-2-yl)-1,4-dihydropyrimidine-5-carboxylate (a Single Stereoisomer)

By utilizing the analogous procedure of Method C, the title compound was synthesized from I-17B-4 and VIa-4 as yellow solids.

LC-MS(ESI): Rₜ=3.29 min, mass calcd. for C₃₂H₄₀FN₅O₆S 641.3, m/z found 642.3 [M+H]⁺.

### Compound I-19A: 3-((4aS*,7aS*)-4-4(S*)-6-(3-fluoro-2-methylphenyl)-5-(methoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

By utilizing the analogous procedure of I-17B, the title compound was synthesized from I-19A-1 as yellow solids.

LC-MS(ESI): Rₜ=2.69 min, mass calcd. for C₂₈H₃₂FN₅O₆S 585.2, m/z found 586.3 [M+H]⁺. ¹H NMR (400 MHz, CDCl₃) δ: 9.52 (brs, 1H), 7.82 (d, J=3.2 Hz, 1H), 7.42 (d, J=3.2 Hz, 1H), 7.09-7.03 (m, 1H), 6.98-6.96 (m, 1H), 6.90 (t, J=9.35 Hz, 1H), 6.01 (s, 1H), 4.68-4.52 (m, 2H), 4.30 (m, 1H), 3.88-3.76 (m, 2H), 3.63 (s, 3H), 3.61-3.37 (m, 3H), 2.98-2.92 (m, 1H), 2.64 (s, 2H), 2.59 (s, 1H), 2.55 (d, J=2.0 Hz, 3H), 1.27 (s, 3H), 1.24 (s, 3H).

### Compound I-20A: 3-((4aS*,7aS*)-4-4(R*)-6-(2-chloro-4-fluorophenyl)-5-(ethoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

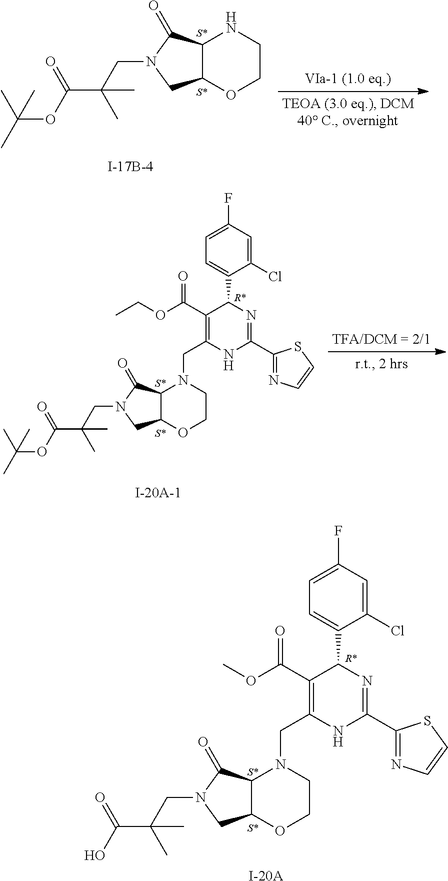 [SMILES: CC(C)(C)OC(=O)C(C)(C)CN1C[C@@H]2OCCN[C@@H]2C1=O ; CCOC(=O)C1=C(CN2CCO[C@H]3CN(CC(C)(C)C(=O)OC(C)(C)C)C(=O)[C@H]32)NC(c2nccs2)=N[C@H]1c1ccc(F)cc1Cl ; COC(=O)C1=C(CN2CCO[C@H]3CN(CC(C)(C)C(=O)O)C(=O)[C@H]32)NC(c2nccs2)=N[C@H]1c1ccc(F)cc1Cl ; "I-20A"]

### Intermediate I-20A-1: ethyl (R*)-6-(((4aS*,7aS*)-6-(3-(tert-butoxy)-2,2-dimethyl-3-oxopropyl)-5-oxohexahydropyrrolo[3,4-b][1,4]oxazin-4(4aH)-yl)methyl)-4-(2-chloro-4-fluorophenyl)-2-(thiazol-2-yl)-1,4-dihydropyrimidine-5-carboxylate (a Single Stereoisomer)

By utilizing the analogous procedure of Method C, the title compound was synthesized from I-17B-4 and VIa-1 as yellow solids.

LC-MS(ESI): Rₜ=2.73 min, mass calcd. for C₃₂H₃₉ClFN₅O₆S 675.2, m/z found 676.3 [M+H]⁺.

### Compound I-20A: 3-((4aS*,7aS*)-4-4(R*)-6-(2-chloro-4-fluorophenyl)-5-(ethoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

By utilizing the analogous procedure of I-17B, the title compound was synthesized from I-20A-1 as yellow solids.

LC-MS(ESI): Rₜ=2.85 min, mass calcd. for C₂₈H₃₁ClFN₅O₆S 619.2, m/z found 620.3 [M+H]⁺. ¹H NMR (400 MHz, DMSO-d₆) δ: 12.27-12.61 (m, 1H), 9.55-9.91 (m, 1H), 7.96 (d, J=3.06 Hz, 1H), 7.89 (d, J=3.18 Hz, 1H), 7.32-7.38 (m, 2H), 7.10 (dt, J=2.69, 8.50 Hz, 1H), 5.97 (s, 1H), 4.44 (s, 2H), 4.09 (br s, 1H), 3.82-3.95 (m, 2H), 3.63-3.78 (m, 2H), 3.50-3.62 (m, 1H), 3.38-3.49 (m, 2H), 3.10-3.21 (m, 1H), 3.04 (d, J=11.00 Hz, 1H), 2.53-2.71 (m, 2H), 0.97-1.05 (m, 9H).

### Compound I-21A: 3-((4aS*,7aS*)-4-4(R*)-6-(2-chloro-3-fluorophenyl)-5-(methoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

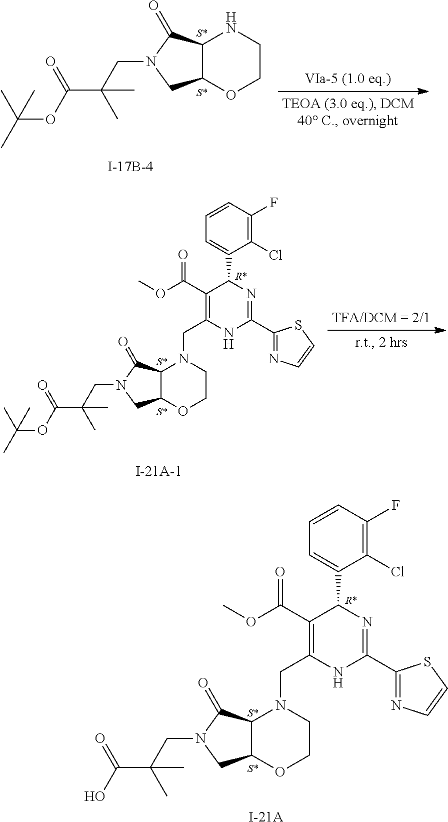 [SMILES: CC(C)(C)OC(=O)C(C)(C)CN1C[C@@H]2OCCN[C@@H]2C1=O ; COC(=O)C1=C(CN2CCO[C@H]3CN(CC(C)(C)C(=O)OC(C)(C)C)C(=O)[C@H]32)NC(c2nccs2)=N[C@H]1c1cccc(F)c1Cl ; COC(=O)C1=C(CN2CCO[C@H]3CN(CC(C)(C)C(=O)O)C(=O)[C@H]32)NC(c2nccs2)=N[C@H]1c1cccc(F)c1Cl ; "I-21A"]

### Intermediate I-21A-1: methyl (R*)-6-(((4aS*,7aS*)-6-(3-(tert-butoxy)-2,2-dimethyl-3-oxopropyl)-5-oxohexahydropyrrolo[3,4-b][1,4]oxazin-4(4aH)-yl)methyl)-4-(2-chloro-3-fluorophenyl)-2-(thiazol-2-yl)-1,4-dihydropyrimidine-5-carboxylate (a Single Stereoisomer)

By utilizing the analogous procedure of Method C, the title compound was synthesized from I-17B-4 and VIa-5 as yellow solids.

LC-MS(ESI): Rₜ=2.57 min, mass calcd. for C₃₁H₃₇ClFN₅O₆S 661.2, m/z found 662.3 [M+H]⁺.

### Compound I-21A: 3-((4aS*,7aS*)-4-4(R*)-6-(2-chloro-3-fluorophenyl)-5-(methoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

By utilizing the analogous procedure of I-17B, the title compound was synthesized from I-21A-1 as yellow solids.

LC-MS(ESI): Rₜ=2.71 min,mass calcd. for C₂₇H₂₉ClFN₅O₆S 605.2, m/z found 606.3 [M+H]⁺. ¹H NMR (400 MHz, DMSO-d₆) δ: 12.15-12.55 (m, 1H), 9.54-9.86 (m, 1H), 7.96 (d, J=3.18 Hz, 1H), 7.89 (d, J=3.18 Hz, 1H), 7.30-7.38 (m, 2H), 7.10 (dt, J=2.63, 8.47 Hz, 1H), 5.96 (s, 1H), 4.44 (br s, 2H), 4.01-4.17 (m, 1H), 3.64-3.80 (m, 2H), 3.50-3.63 (m, 1H), 3.45 (s, 3H), 3.38-3.48 (m, 2H), 3.15 (d, J=13.69 Hz, 1H), 3.05 (d, J=11.00 Hz, 1H), 2.53-2.72 (m, 2H), 1.03 (s, 3H), 1.00 (s, 3H).

### Compound I-22A: 3-((4aS*,7aS*)-4-4(R*)-6-(2-chloro-3,4-difluorophenyl)-5-(methoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

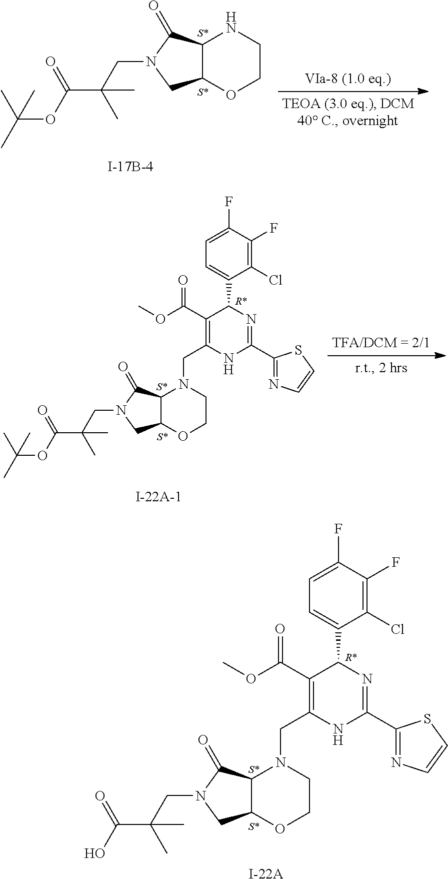 [SMILES: CC(C)(C)OC(=O)C(C)(C)CN1C[C@@H]2OCCN[C@@H]2C1=O ; COC(=O)C1=C(CN2CCO[C@H]3CN(CC(C)(C)C(=O)OC(C)(C)C)C(=O)[C@H]32)NC(c2nccs2)=N[C@H]1c1ccc(F)c(F)c1Cl ; COC(=O)C1=C(CN2CCO[C@H]3CN(CC(C)(C)C(=O)O)C(=O)[C@H]32)NC(c2nccs2)=N[C@H]1c1ccc(F)c(F)c1Cl ; "I-22A"]

### Intermediate I-22A-1: methyl (R*)-6-4(4aS*,7aS*)-6-(3-(tert-butoxy)-2,2-dimethyl-3-oxopropyl)-5-oxohexahydropyrrolo[3,4-b][1,4]oxazin-4(4aH)-yl)methyl)-4-(2-chloro-3,4-difluorophenyl)-2-(thiazol-2-yl)-1,4-dihydropyrimidine-5-carboxylate (a Single Stereoisomer)

By utilizing the analogous procedure of Method C, the title compound was synthesized from I-17B-4 and VIa-8 as yellow solids.

LC-MS(ESI): Rₜ=3.37 min, mass calcd. for C₃₁H₃₆ClF₂N₅O₆S 679.2, m/z found 680.3 [M+H]⁺.

### Compound I-22A: 3-((4aS*,7aS*)-4-4(R*)-6-(2-chloro-3,4-difluorophenyl)-5-(methoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

By utilizing the analogous procedure of I-17B, the title compound was synthesized from I-22A-1 as yellow solids.

LC-MS(ESI): Rₜ=2.81 min, mass calcd. for C₂₇H₂₈ClF2N₅O₆S 623.1, m/z found 624.2 [M+1-1]⁺. ¹H NMR (400 MHz, METHANOL-d₄) δ: 7.94 (d, J=3.18 Hz, 1H), 7.74 (d, J=3.18 Hz, 1H), 7.17-7.24 (m, 2H), 6.14 (s, 1H), 4.51-4.66 (m, 2H), 4.28 (t, J=3.55 Hz, 1H), 3.75-3.88 (m, 2H), 3.66-3.72 (m, 1H), 3.56-3.65 (m, 5H), 3.32-3.40 (m, 2H), 2.83-2.92 (m, 1H), 2.61-2.69 (m, 1H), 1.20 (s, 3H), 1.17 (s, 3H).

### Compound I-23A: 3-((4aS*,7aS*)-4-4(R*)-6-(2-chloro-3,4-difluorophenyl)-5-(ethoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

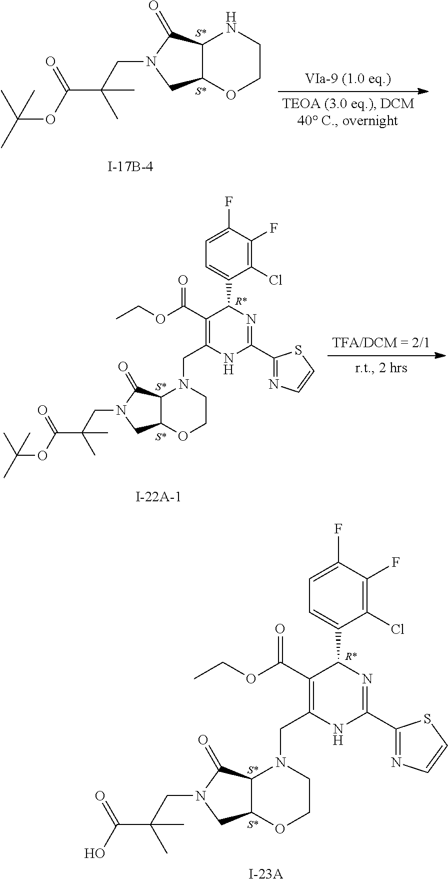 [SMILES: CC(C)(C)OC(=O)C(C)(C)CN1C[C@@H]2OCCN[C@@H]2C1=O ; CCOC(=O)C1=C(CN2CCO[C@H]3CN(CC(C)(C)C(=O)OC(C)(C)C)C(=O)[C@H]32)NC(c2nccs2)=N[C@H]1c1ccc(F)c(F)c1Cl ; CCOC(=O)C1=C(CN2CCO[C@H]3CN(CC(C)(C)C(=O)O)C(=O)[C@H]32)NC(c2nccs2)=N[C@H]1c1ccc(F)c(F)c1Cl ; "I-23A"]

### Intermediate I-23A-1: ethyl (R*)-6-(((4aS*,7aS*)-6-(3-(tert-butoxy)-2,2-dimethyl-3-oxopropyl)-5-oxohexahydropyrrolo[3,4-b][1,4]oxazin-4(4aH)-yl)methyl)-4-(2-chloro-3,4-difluorophenyl)-2-(thiazol-2-yl)-1,4-dihydropyrimidine-5-carboxylate (a Single Stereoisomer)

By utilizing the analogous procedure of Method C, the title compound was synthesized from I-17B-4 and VIa-9 as yellow solids.

LC-MS(ESI): Rₜ=2.73 min, mass calcd. for C₃₂H₃₈ClF₂N₅O₆S 693.2, m/z found 694.3 [M+H]⁺.

### Compound I-23A: 3-((4aS*,7aS*)-4-4(R*)-6-(2-chloro-3,4-difluorophenyl)-5-(ethoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

By utilizing the analogous procedure of I-17B, the title compound was synthesized from I-23A-1 as yellow solids.

LC-MS(ESI): Rₜ=2.90 min, mass calcd. for C₂₈H₃₀ClF2N₅O₆S 637.1, m/z found 638.2 [M+H]⁺. ¹H NMR (400 MHz, METHANOL-d₄) δ: ppm 7.94 (d, J=3.18 Hz, 1H), 7.74 (d, J=3.06 Hz, 1H), 7.17-7.25 (m, 2H), 6.14 (s, 1H), 4.51-4.66 (m, 2H), 4.28 (t, J=3.55 Hz, 1H), 4.05 (q, J=7.09 Hz, 2H), 3.82 (td, J=10.42, 2.14 Hz, 2H), 3.69 (d, J=3.79 Hz, 1H), 3.55-3.63 (m, 2H), 3.33-3.40 (m, 1H), 3.27-3.30 (m, 1H), 2.88 (br s, 1H), 2.60-2.71 (m, 1H), 1.12-1.23 (m, 9H).

### Compound I-24A: 3-((4aS*,7aS*)-4-4(R*)-6-(2-chloro-3-fluorophenyl)-5-(methoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

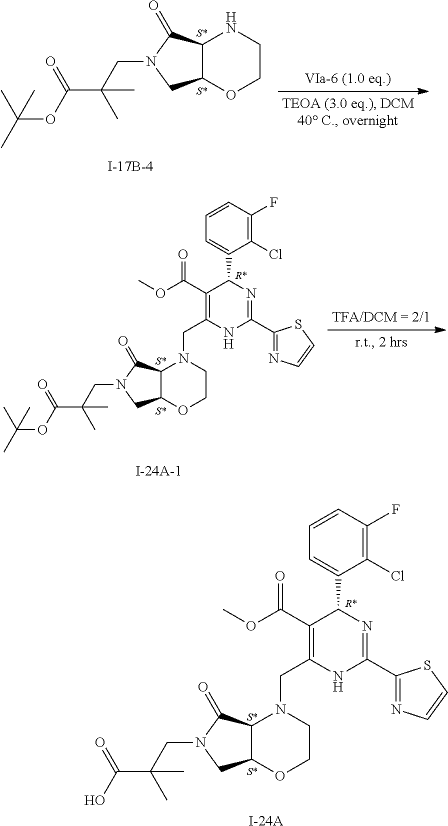 [SMILES: CC(C)(C)OC(=O)C(C)(C)CN1C[C@@H]2OCCN[C@@H]2C1=O ; COC(=O)C1=C(CN2CCO[C@H]3CN(CC(C)(C)C(=O)OC(C)(C)C)C(=O)[C@H]32)NC(c2nccs2)=N[C@H]1c1cccc(F)c1Cl ; COC(=O)C1=C(CN2CCO[C@H]3CN(CC(C)(C)C(=O)O)C(=O)[C@H]32)NC(c2nccs2)=N[C@H]1c1cccc(F)c1Cl ; "I-24A"]

### Intermediate I-24A-1: methyl (R*)-6-(((4aS*,7aS*)-6-(3-(tert-butoxy)-2,2-dimethyl-3-oxopropyl)-5-oxohexahydropyrrolo[3,4-b][1,4]oxazin-4(4aH)-yl)methyl)-4-(2-chloro-3-fluorophenyl)-2-(thiazol-2-yl)-1,4-dihydropyrimidine-5-carboxylate (a Single Stereoisomer)

By utilizing the analogous procedure of Method C, the title compound was synthesized from I-17B-4 and VIa-6 as yellow solids.

LC-MS(ESI): Rₜ=3.30 min, mass calcd. for C₃₁H₃₇ClFN₅O₆S 661.2, m/z found 662.3 [M+H]⁺.

### Compound I-24A: 3-((4aS*,7aS*)-4-4(R*)-6-(2-chloro-3-fluorophenyl)-5-(methoxycarbonyl)-2-(thiazol-2-yl)-3,6-dihydropyrimidin-4-yl)methyl)-5-oxohexahydropyrrolo[3,4-b][1,4]oxazin-6(2H)-yl)-2,2-dimethylpropanoic acid (a Single Stereoisomer)

By utilizing the analogous procedure of I-17B, the title compound was synthesized from I-24A-1 as yellow solids.

LC-MS(ESI): Rₜ=2.70 min, mass calcd. for C₂₇H₂₉ClFN₅O₆S 605.1, m/z found 606.2 [M+H]⁺. ¹H NMR (400 MHz, CDCl₃) δ: ppm 9.45-9.64 (m, 1H), 7.80-7.94 (m, 1H), 7.40-7.51 (m, 1H), 7.08-7.21 (m, 2H), 6.97-7.08 (m, 1H), 6.18-6.30 (m, 1H), 4.59-4.74 (m, 1H), 4.45-4.57 (m, 1H), 4.25-4.36 (m, 1H), 3.72-3.94 (m, 2H), 3.62 (s, 3H), 3.39-3.60 (m, 5H), 2.87-2.99 (m, 1H), 2.51-2.63 (m, 1H), 1.22-1.33 ppm (m, 6H).

Below compound from patent WO01/68641 (compound 72 in the patent) was selected as reference 1. Chemical structure is shown below.

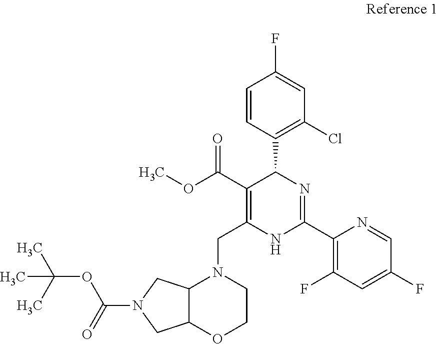 [SMILES: COC(=O)C1=C(CN2CCOC3CN(C(=O)OC(C)(C)C)CC32)NC(c2ncc(F)cc2F)=N[C@H]1c1ccc(F)cc1Cl]

### Reference 1A (a Single Stereoisomer) and Reference 1B (a Single Stereoisomer): (4aR*,7aR*)-tert-butyl 4-(((R*)-6-(2-chloro-4-fluorophenyl)-2-(3,5-difluoropyridin-2-yl)-5-(methoxycarbonyl)-3,6-dihydropyrimidin-4-yl)methyl)hexahydropyrrolo[3,4-b][1,4]oxazine-6(2H)-carboxylate (a Single Stereoisomer) and (4aS*,7aS*)-tert-butyl 4-(((R*)-6-(2-chloro-4-fluorophenyl)-2-(3,5-difluoropyridin-2-yl)-5-(methoxycarbonyl)-3,6-dihydropyrimidin-4-yl)methyl)hexahydropyrrolo[3,4-b][1,4]oxazine-6(2H)-carboxylate (a Single Stereoisomer)

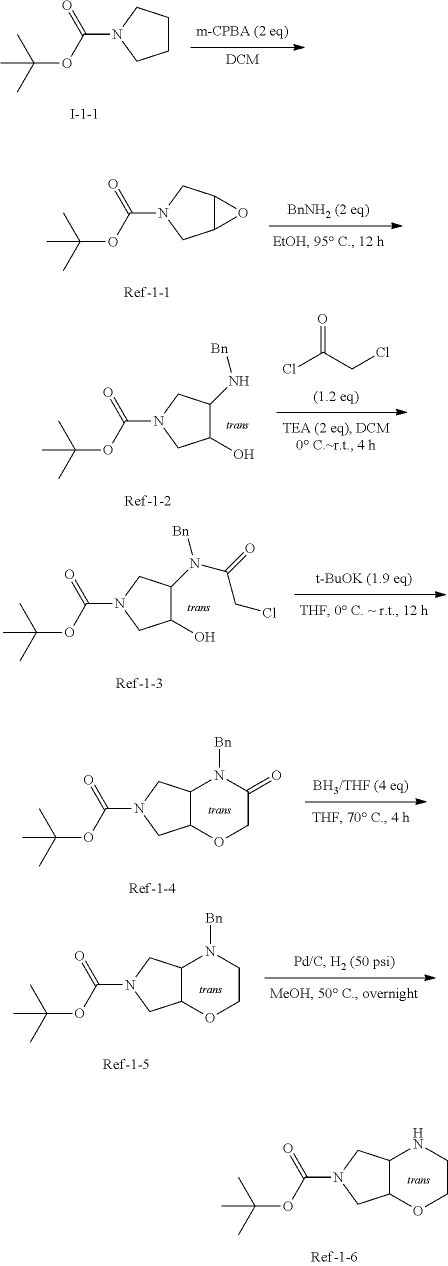

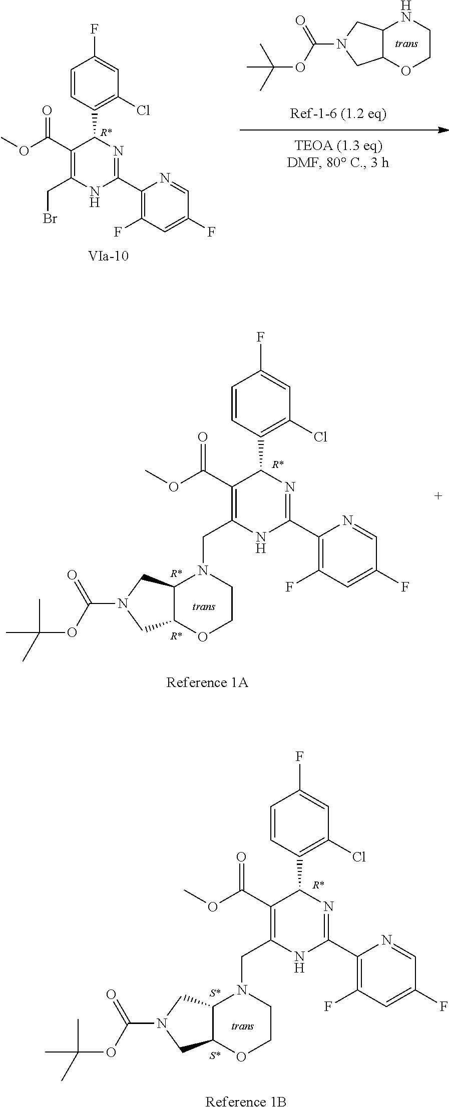

### Intermediate Ref-1-1: tert-butyl 6-oxa-3-azabicyclo[3.1.0]hexane-3-carboxylate

To a solution of tert-butyl 2,5-dihydro-1H-pyrrole-1-carboxylate I-1-1(20 g, 118 mmol) in dichloromethane (300 mL) was added 3-chloroperoxybenzoic acid (41 g, 238 mmol) at room temperature. After stirred at room temperature for 12 hours, the reaction mixture was quenched with saturated sodium sulfite aqueous solution (500 mL), washed with saturated sodium bicarbonate aqueous solution (500 mL) for three times, dried over Na₂SO₄(s) and filtered. The filtrate was concentrated under reduced pressure to give the title compound (19 g, 80% purity from ¹H NMR, 69% yield) as yellow oil. ¹H NMR (400 MHz, CD₃OD) δ 3.82 (d, J=12.8 Hz, 1H), 3.74 (d, J=12.8 Hz, 1H), 3.68 (d, J=2.8 Hz, 2H), 3.36-3.30 (m, 2H), 1.45 (s, 9H).

### Intermediate Ref-1-2: (trans)-tert-butyl 3-(benzylamino)-4-hydroxypyrrolidine-1-carboxylate

To a solution of tert-butyl 6-oxa-3-azabicyclo[3.1.0]hexane-3-carboxylate Ref-1-1(21 g, 80% purity, 90.7 mmol) in ethanol (200 mL) was added phenylmethanamine (20 mL, 183 mmol) at room temperature. After stirred at 95° C. for 12 hours, the reaction mixture was concentrated to give a residue. The residue was diluted with a solution of petroleum ether (50 mL) and ethyl acetate (50 mL). The resulting suspension was stirred at room temperature for 1 hour. The precipitate was collected by filtration to give the title compound (21 g, 95% purity from ¹H NMR, 75% yield) as white solids. ¹H NMR (400 MHz, DMSO-d₆) δ 7.36-7.28 (m, 4H), 7.23-7.20 (m, 1H), 5.00 (br s, 1H), 3.97 (s, 1H), 3.70 (s, 2H), 3.46-3.31 (m, 2H), 3.13-3.06 (m, 2H), 2.95-2.93 (m, 1H), 1.39 (s, 9H).

### Intermediate Ref-1-3: (trans)-tert-butyl 3-(N-benzyl-2-chloroacetamido)-4-hydroxypyrrolidine-1-carboxylate

To a solution of (trans)-tert-butyl 3-(benzylamino)-4-hydroxypyrrolidine-1-carboxylate Ref-1-2 (21 g, 95% purity, 68.2 mmol) and triethylamine (19 mL, 137 mmol) in dichloromethane (200 mL) was added 2-chloroacetyl chloride (6.6 mL, 82.9 mmol) dropwise at 0° C. After stirred at room temperature for 4 hours, the mixture was diluted with water (100 mL) and extracted with ethyl acetate (100 mL) for three times. The combined organic layers were dried over Na₂SO₄₍ₛ₎ and filtered. The filtrate was concentrated and purified by silica gel column chromatography (petroleum ether:ethyl acetate=1:0 to 8:1) to give the title compound (11.5 g, 80% purity from ¹H NMR, 37% yield) as yellow solids. ¹H NMR (400 MHz, DMSO-d₆) δ 7.41-7.15 (m, 5H), 5.61-5.58 (m, 0.7H), 5.37 (s, 0.3H), 4.87-4.77 (m, 1.3H), 4.51 (d, J=14.0 Hz, 0.7H), 4.40-4.21 (m, 3.3H), 3.52-3.38 (m, 2H), 3.18-2.93 (m, 2H), 1.36-1.31 (m, 9H).

### Intermediate Ref-1-4: (trans)-tert-butyl 4-benzyl-3-oxohexahydropyrrolo[3,4-b][1,4]oxazine-6(2H)-carboxylate

To a solution of (trans)-tert-butyl 3-(N-benzyl-2-chloroacetamido)-4-hydroxy pyrrolidine-1-carboxylate Ref-1-3 (11.5 g, 80% purity, 24.9 mmol) in tetrahydrofuran (100 mL) at 0° C. was added potassium tert-butoxide (5.3 g, 47.2 mmol). After stirred at room temperature for 12 hours, the reaction mixture was diluted with water (100 mL) and extracted with ethyl acetate (100 mL) for three times. The combined organic layers were dried over Na₂SO₄₍ₛ₎ and filtered. The filtrate was concentrated under reduced pressure to give the title compound (2.8 g, 95% purity from ¹H NMR, 32% yield) as white solids.

¹H NMR (300 MHz, DMSO-d₆) δ 7.41-7.36 (m, 2H), 7.33-7.26 (m, 3H), 4.83-4.74 (m, 1H), 4.40 (s, 2H), 4.38-4.32 (m, 1H), 4.25-4.16 (m, 1H), 3.77-3.57 (m, 3H), 3.13-2.88 (m, 2H), 1.39 (s, 9H).

### Intermediate Ref-1-5: (trans)-tert-butyl 4-benzylhexahydropyrrolo[3,4-b][1,4]oxazine-6(2H)-carboxylate

To a mixture of (trans)-tert-butyl 4-benzyl-3-oxohexahydropyrrolo[3,4-b][1,4]oxazine-6(2H)-carboxylate Ref-1-4 (500 mg, 90% purity, 1.35 mmol) in tetrahydrofuran (10 mL) was added 1 M borane-tetrahydrofuran complex (5.5 mL, 5.5 mmol). After stirred at 70° C. for 4 hours, the reaction mixture was cooled down to room temperature, quenched with saturated ammonium chloride aqueous solution (20 mL) and extracted with ethyl acetate (30 mL) twice. The combine organic layers were washed with brine (50 mL), dried over Na₂SO₄₍ₛ₎ and filtered. The filtrate was concentrated and purified by silica gel column chromatography (dichloromethane:methanol=20:1) to give the title compound (400 mg, 90% purity from ¹H NMR, 84% yield) as yellow oil. ¹H NMR (300 MHz, CDCl₃) δ 7.41-7.32 (m, 5H), 3.94 (dd, J=11.4 Hz, 3.0 Hz, 1H), 3.81-3.61 (m, 5H), 3.31-3.17 (m, 2H), 3.07-3.00 (m, 1H), 2.73 (d, J=11.7 Hz, 1H), 2.46-2.33 (m, 1H), 2.26-2.15 (m, 1H), 1.49 (s, 9H).

### Intermediate Ref-1-6: (trans)-tert-butyl hexahydropyrrolo[3,4-b][1,4]oxazine-6(2H)-carboxylate

To a mixture of (trans)-tert-butyl 4-benzylhexahydropyrrolo[3,4-b][1,4]oxazine-6(2H)-carboxylate Ref-1-5 (1.7 g, 90% purity, 4.81 mmol) in methanol (30 mL) was added 10% wt. palladium on charcoal (340 mg). After stirred at 50° C. under hydrogen atmosphere (50 psi) overnight, the reaction mixture was filtered. The filtrate was concentrated and purified by silica gel column chromatography (dichloromethane:methanol=20:1) to give the title compound (1.1 g, 90% purity from ¹H NMR, 90% yield) as yellow oil. ¹H NMR (400 MHz, CDCl₃) δ 3.96-3.93 (m, 1H), 3.75-3.56 (m, 3H), 3.50-3.40 (m, 1H), 3.15-2.77 (m, 5H), 1.46 (s, 9H).

### Compounds Reference 1A and Reference 1B: (4aR*,7aR*)-tert-butyl 4-(((R*)-6-(2-chlo ro-4-fluo rophenyl)-2-(3,5-difluoro pyridin-2-yl)-5-(methoxycarbonyl)-3,6-dihydropyrimidin-4-yl)methyphexahydropyrrolo[3,4-b][1,4]oxazine-6(2H)-carboxylate (4aS*,7aS*)-tert-butyl 4-(((R*)-6-(2-chloro-4-fluorophenyl)-2-(3,5-difluoropyridin-2-yl)-5-(methoxycarbonyl)-3,6-dihydropyrimidin-4-yl)methyphexahydropyrrolo[3,4-b][1,4]oxazine-6(2H)-carboxylate

By utilizing the analogous procedure of Method C, the filtrate of the title compounds were synthesized from Ref-1-6 and VIa-10 as yellow solids. The filtrate was purified by Prep. HPLC (Column: X-bridge C18 (5 um 19*150 mm); Mobile Phase A: water (+0.1% ammonium bicarbonate), Mobile Phase B: acetonitrile, UV: 214 nm, Flow rate: 15 mL/min, Gradient: 40-90% (% B)) to give Reference 1A (25 mg, 95.4% purity, 32% yield) and Reference 1B (28 mg, 96.3% purity, 36% yield) as white solids. Reference 1A: LC-MS (ESI): R_{T}=3.105 min, mass calcd. for C₂₉H₃₁ClF₃N₅O₅ 621.2, m/z found 622.3 [M+H]⁺. ¹H NMR (400 MHz, CD₃OD) δ 8.52 (s, 1H), 7.72-7.68 (m, 1H), 7.41-7.38 (m, 1H), 7.22 (d, J=8.8 Hz, 1H), 7.05-7.01 (m, 1H), 6.22 (s, 1H), 4.19-4.02 (m, 2H), 3.94-3.86 (m, 2H), 3.73-3.67 (m, 3H), 3.62 (s, 3H), 3.17-2.94 (m, 3H), 2.63-2.55 (m, 2H), 1.45 (s, 4H), 1.42 (s, 5H).

Reference 1B: LC-MS (ESI): R_{T}=3.255 min, mass calcd. for C₂₉H₃₁ClF3N₅O₅ 621.2, m/z found 622.3 [M+H]⁺. ¹H NMR (400 MHz, CD₃OD) δ 8.51 (s, 1H), 7.80-7.69 (m, 1H), 7.50-7.40 (m, 1H), 7.31-7.21 (m, 1H), 7.12-6.99 (m, 1H), 6.33 (s, 0.2H), 6.24 (s, 0.8H), 4.21-4.13 (m, 1H), 4.06-3.88 (m, 4H), 3.83-3.70 (m, 2H), 3.62 (s, 3H), 3.20-3.09 (m, 2H), 2.78 (t, J=10.4 Hz, 1H), 2.69-2.60 (m, 1H), 2.49-2.39 (m, 1H), 1.48 (s, 9H).

### Reference 1C (a Single Stereoisomer) and Reference 1D (a Single Stereoisomer): (4aR*,7aR*)-tert-butyl 4-4(S*)-6-(2-chloro-4-fluorophenyl)-2-(3,5-difluoropyridin-2-yl)-5-(methoxycarbonyl)-3,6-dihydropyrimidin-4-yl)methyl)hexahydropyrrolo[3,4-b][1,4]oxazine-6(2H)-carboxylate (a Single Stereoisomer) and (4aS*,7aS*)-tert-butyl 4-4(S*)-6-(2-chloro-4-fluorophenyl)-2-(3,5-difluoropyridin-2-yl)-5-(methoxycarbonyl)-3,6-dihydropyrimidin-4-yl)methyl)hexahydropyrrolo[3,4-b][1,4]oxazine-6(2H)-carboxylate (a Single Stereoisomer)

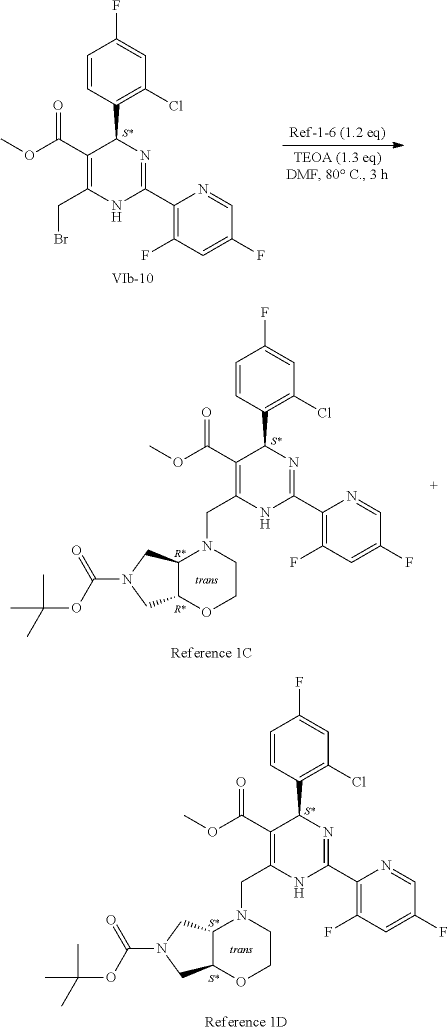 [SMILES: COC(=O)C1=C(CBr)NC(c2ncc(F)cc2F)=N[C@@H]1c1ccc(F)cc1Cl ; COC(=O)C1=C(CN2CCO[C@@H]3CN(C(=O)OC(C)(C)C)C[C@H]32)NC(c2ncc(F)cc2F)=N[C@@H]1c1ccc(F)cc1Cl ; "Reference 1C" ; COC(=O)C1=C(CN2CCO[C@H]3CN(C(=O)OC(C)(C)C)C[C@@H]32)NC(c2ncc(F)cc2F)=N[C@@H]1c1ccc(F)cc1Cl ; "Reference 1D"]

By utilizing the analogous procedure of Method C, the filtrate of the title compounds were synthesized from Ref-1-6 and Vlb-10 as yellow solids. The filtrate was concentrated and purified by Prep. HPLC (Column: X-bridge C18 (5 um 19*150 mm); Mobile Phase A: water (+0.1% ammonium bicarbonate), Mobile Phase B: acetonitrile, UV: 214 nm, Flow rate: 15 mL/min, Gradient: 40-90% (% B)) to give Reference 1C (16 mg, 99.3% purity, 21.3% yield) and Reference 1D (6 mg, 98.1% purity, 7.9% yield) as white solids.

Reference 1C: LC-MS (ESI): R_{T}=3.514 min, mass calcd. for C₂₉H₃₁ClF₃N₅O₅ 621.2, m/z found 622.2 [M+H]⁺. ¹H NMR (400 MHz, CD₃OD) δ 8.51 (s, 1H), 7.74-7.68 (m, 1H), 7.41-7.37 (m, 1H), 7.21 (d, J=8.4 Hz, 1H), 7.05-7.01 (m, 1H), 6.21 (s, 1H), 4.18-4.02 (m, 2H), 3.94-3.85 (m, 2H), 3.75-3.65 (m, 3H), 3.63 (s, 1H), 3.62 (s, 2H), 3.17-2.93 (m, 3H), 2.65-2.52 (m, 2H), 1.44 (s, 4H), 1.41 (s, 5H).

Reference 1D: LC-MS (ESI): R_{T}=3.697 min, mass calcd. for C₂₉H₃₁ClF₃N₅O₅ 621.2, m/z found 622.2 [M+H]⁺. ¹H NMR (400 MHz, CD₃OD) δ 8.51 (s, 1H), 7.75-7.70 (m, 1H), 7.44-7.42 (m, 1H), 7.25-7.22 (m, 1H), 7.12-7.02 (m, 1H), 6.26 (s, 1H), 4.22-4.14 (m, 1H), 3.99-3.84 (m, 4H), 3.80-3.72 (m, 2H), 3.62 (s, 3H), 3.19-3.13 (m, 2H), 2.78 (t, J=10.8 Hz, 1H), 2.69-2.63 (m, 1H), 2.51-2.43 (m, 1H), 1.48 (s, 9H).

### Reference 1E: tert-butyl (4aR*,7aS*)-4-4(R*)-6-(2-chloro-4-fluorophenyl)-2-(3,5-difluoropyridin-2-yl)-5-(methoxycarbonyl)-3,6-dihydropyrimidin-4-yl)methyl)hexahydropyrrolo[3,4-b][1,4]oxazine-6(2H)-carboxylate (a Single Stereoisomer)

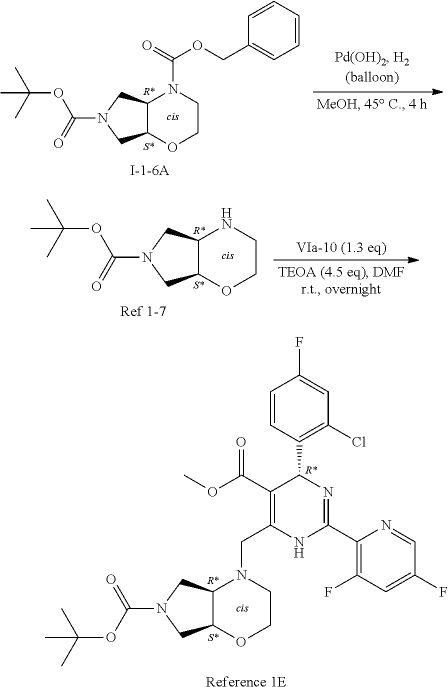

### Intermediate Ref-1-7: tert-butyl (4aR*,7aS*)-hexahydropyrrolo[3,4-b][1,4]oxazine-6(2H)-carboxylate

To a solution of I-1-6A (150 mg, 90% purity, 0.372 mmol) in methanol (20 mL) was added 10% wt. palladium hydroxide on charcoal (90 mg) at room temperature. After stirred at 45° C. for 4 hours under hydrogen atmosphere (balloon), the mixture was filtered. The filtrate was concentrated to give the title compound (85 mg, 90% purity from ¹I-1 NMR, 90% yield) as colorless oil.

LC-MS (ESI): R_{T}=1.25 min, mass calcd. for C₁₁H₂₀N₂O₃ 228.1, m/z found 229.1 [M+H]⁺. ¹H NMR (400 MHz, CDCl₃) δ 3.99-3.93 (m, 1H), 3.84 (td, J=11.2 Hz, 2.8 Hz, 1H), 3.61-3.52 (m, 2H), 3.47-3.35 (m, 4H), 3.15-3.10 (m, 1H), 2.66 (td, J=13.2 Hz, 2.4 Hz, 1H), 1.46-1.45 (m, 9H).

### Reference 1E: tert-butyl (4aR*,7aS*)-4-4(R*)-6-(2-chloro-4-fluorophenyl)-2-(3,5-difluoropyridin-2-yl)-5-(methoxycarbonyl)-3,6-dihydropyrimidin-4-yl)methyphexahydropyrrolo[3,4-b][1,4]oxazine-6(2H)-carboxylate (a Single Stereoisomer)

By utilizing the analogous procedure of Method C, the title compound was synthesized from Ref-1-7 and VIa-10 as yellow solids.

LC-MS (ESI): R_{T}=3.329 min, mass calcd. for C₂₈H₃₁ClF₃N₅O₅ 621.2, m/z found 622.3 [M+H]⁺. ¹H NMR (400 MHz, CD₃OD) δ 8.53-8.51 (m, 1H), 7.75-7.69 (m, 1H), 7.44-7.38 (m, 1H), 7.23-7.22 (m, 1H), 7.06-6.99 (m, 1H), 6.23 (s, 0.4H), 6.22 (s, 0.6H), 4.35-4.21 (m, 2H), 4.03-3.89 (m, 2H), 3.84-3.74 (m, 1H), 3.62 (s, 3H), 3.58-3.51 (m, 1.5H), 3.48-3.31 (m, 3.5H), 2.96-2.82 (m, 1H), 2.67-2.61 (m, 0.6H), 2.50-2.47 (m, 0.4H), 1.46 (s, 4H), 1.43 (s, 2.5H), 1.37 (s, 2.5H).

### Reference 1F: tert-butyl (4aR*,7aS*)-4-4(S*)-6-(2-chloro-4-fluorophenyl)-2-(3,5-difluoropyridin-2-yl)-5-(methoxycarbonyl)-3,6-dihydropyrimidin-4-yl)methyphexahydropyrrolo[3,4-b][1,4]oxazine-6(2H)-carboxylate (a Single Stereoisomer)

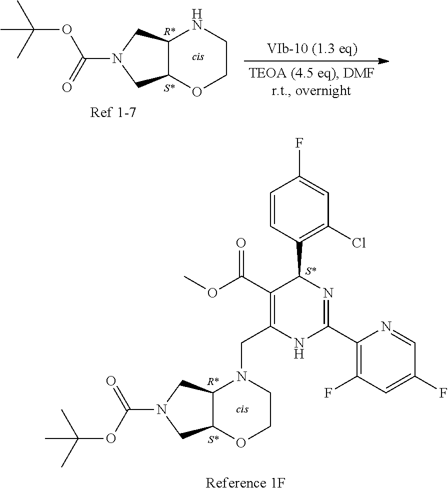

By utilizing the analogous procedure of Method C, the title compound was synthesized from Ref-1-7 and VIb-10 as yellow solids.

LC-MS (ESI): R_{T}=3.766 min, mass calcd. for C₂₈H₃₁ClF3N₅O₅ 621.2, m/z found 622.2 [M+H]⁺. ¹H NMR (400 MHz, CD₃OD) δ 8.53-8.51 (m, 1H), 7.75-7.68 (m, 1H), 7.44-7.37 (m, 1H), 7.23-7.20 (m, 1H), 7.06-7.00 (m, 1H), 6.24 (s, 0.5H), 6.22 (s, 0.5H), 4.35-4.19 (m, 2H), 4.03-3.90 (m, 2H), 3.84-3.74 (m, 1H), 3.62 (s, 3H), 3.58-3.52 (m, 1.7H), 3.48-3.37 (m, 3.3H), 2.96-2.82 (m, 1H), 2.67-2.59 (m, 0.5H), 2.50-2.47 (m, 0.5H), 1.46 (s, 4H), 1.43 (s, 2.5H), 1.37 (s, 2.5H).

### Reference 1G: (4aS*,7aR*)-tert-butyl 4-(((R*)-6-(2-chloro-4-fluorophenyl)-2-(3,5-difluoropyridin-2-yl)-5-(methoxycarbonyl)-3,6-dihydropyrimidin-4-yl)methyl)hexahydropyrrolo[3,4-b][1,4]oxazine-6(2H)-carboxylate (a Single Stereoisomer)

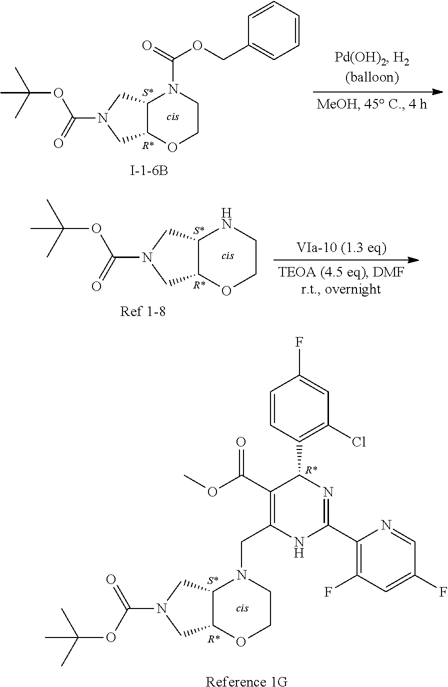

### Intermediate Ref-1-8: (4 aS*,7aR*)-tert-butyl hexahydropyrrolo[3,4-b][1,4]oxazine-6(2H)-carboxylate

To a solution of I-1-6B (150 mg, 90% purity, 0.372 mmol) in methanol (20 mL) was added 10% wt. palladium hydroxide on charcoal (90 mg) at room temperature. After stirred at 45° C. for 4 hours under hydrogen atmosphere (balloon), the mixture was filtered. The filtrate was concentrated to give the title compound (85 mg, 90% purity from ¹I-1 NMR, 90% yield) as colorless oil.

LC-MS (ESI): R_{T}=1.25 min, mass calcd. for C₁₁H₂₀N₂O₃ 228.1, m/z found 229.1 [M+H]⁺. ¹H NMR (400 MHz, CDCl₃) δ 3.99-3.93 (m, 1H), 3.84 (td, J=11.2 Hz, 2.8 Hz, 1H), 3.61-3.52 (m, 2H), 3.47-3.35 (m, 4H), 3.15-3.10 (m, 1H), 2.66 (td, J=13.2 Hz, 2.4 Hz, 1H), 1.46-1.45 (m, 9H).

### Reference 1G: (4aS*,7aR*)-tert-butyl 4-(((R*)-6-(2-chloro-4-fluorophenyl)-2-(3,5-difluoropyridin-2-yl)-5-(methoxycarbonyl)-3,6-dihydropyrimidin-4-yl)methyl)hexahydropyrrolo[3,4-b][1,4]oxazine-6(2H)-carboxylate (a Single Stereoisomer)

By utilizing the analogous procedure of Method C, the title compound was synthesized from Ref-1-8 and VIa-10 as yellow solids.

LC-MS (ESI): R_{T}=4.601 min, mass calcd. for C₂₉H₃₁ClF₃N₅O₅ 621.2, m/z found 622.3 [M+H]⁺. ¹H NMR (400 MHz, CD₃OD) δ 8.52 (d, J=6.4 Hz, 1H), 7.76-7.69 (m, 1H), 7.44-7.37 (m, 1H), 7.24-7.20 (m, 1H), 7.06-7.00 (m, 1H), 6.23 (s, 1H), 4.28-4.21 (m, 2H), 4.04-3.93 (m, 2H), 3.79-3.70 (m, 1H), 3.62 (s, 3H), 3.59-3.56 (m, 1.3H), 3.51-3.38 (m, 3.7H), 2.96-2.91 (m, 1H), 2.68-2.61 (m, 1H), 1.43 (s, 4.3H), 1.37 (s, 4.7H).

### Reference 1H: (4aS*,7aR*)-tert-butyl 4-(((S*)-6-(2-chloro-4-fluorophenyl)-2-(3,5-difluoropyridin-2-yl)-5-(methoxycarbonyl)-3,6-dihydropyrimidin-4-yl)methyl)hexahydropyrrolo[3,4-b][1,4]oxazine-6(2H)-carboxylate (a Single Stereoisomer)

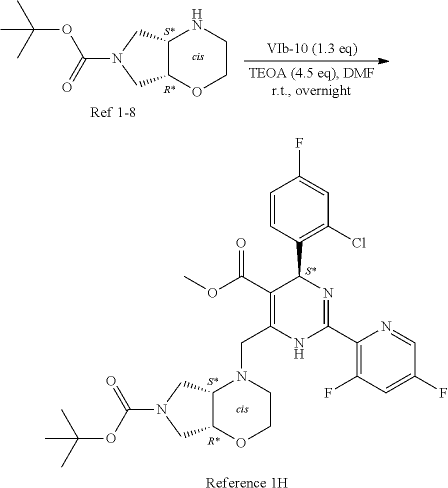

By utilizing the analogous procedure of Method C, the title compound was synthesized from Ref-1-8 and VIb-10 as yellow solids.

LC-MS (ESI): R_{T}=3.662 min, mass calcd. for C₂₉H₃₁ClF₃N₅O₅ 621.2, m/z found 622.3 [M+H]⁺. ¹H NMR (400 MHz, CD₃OD) δ 8.51 (s, 1H), 7.74-7.69 (m, 1H), 7.42-7.38 (m, 1H), 7.22 (dd, J=8.8 Hz, 2.8 Hz, 1H), 7.04-6.99 (m, 1H), 6.24 (s, 1H), 4.35-4.29 (m, 1H), 4.21-4.19 (m, 1H), 4.01-3.90 (m, 2H), 3.84-3.79 (m, 1H), 3.62 (s, 3H), 3.59-3.52 (m, 2H), 3.49-3.47 (m, 3H), 2.89-2.82 (m, 1H), 2.49 (d, J=12.0 Hz, 1H), 1.46 (s, 9H).

### Example 2: Anti-Viral Assay in HepG2.2.15 Cells

### Materials and Equipments

1) Cell Line

HepG2.2.15 (the HepG2.2.15 cell line can be produced by transfection of the HepG2 cell line as described in Sells, Chen, and Acs 1987 (Proc. Natl. Acad. Sci. USA 84: 1005-1009), and the HepG2 cell line is available from ATCC® under number HB-8065™).

2) Reagents

### DMEM/F12 (INVITROGEN-11330032)

### FBS (GIBCO-10099-141)

Dimethyl sulfoxide(DMSO) (SIGMA-D2650)
Penicillin-streptomycin solution (HYCLONE-SV30010)

### NEAA (INVITROGEN-1114050)

### L-Glutamine (INVITROGEN-25030081)

Geneticin Selective Antibiotic (G418, 500 mg/ml) (INVITROGEN-10131027)
Trypsinase digestion solution (INVITROGEN-25300062)

### CCK8 (BIOLOTE-35004)

### QIAamp 96 DNA Blood Kit (12) (QIAGEN-51162)

### FastStart Universal Probe Mast Mix (ROCHE-04914058001)

3) Consumables

96-well cell culture plate (COSTAR-3599)
Micro Amp Optical 96-well reaction plate (APPLIED BIOSYSTEMS-4306737)
Micro Amp Optical 384-well reaction plate (APPLIED BIOSYSTEMS)

4) Equipment

Plate reader (MOLECULAR DEVICES, SPECTRAMAX M2e)

### Centrifuge (BECKMAN, ALLEGRA-X15R)

Real Time PCR system (APPLIED BIOSYSTEMS, QUANTSTUDIO 6)
Real Time PCR system (APPLIED BIOSYSTEMS, 7900HT)

### Methods

1) Anti-HBV Activity and Cytotoxicity Determination

HepG2.2.15 cells were plated into 96-well plate in 2% FBS culture medium at the density of 40,000 cells/well and 5,000 cells/well for HBV inhibitory activity and cytotoxicity determination, respectively. After incubation at 37° C., 5% CO2 overnight, cells were treated with medium containing compounds for 6 days with medium and compounds refreshed after 3 days of treatment. Each compound was tested in a 1:3 serial dilutions at 8 different concentrations in triplicate. The highest concentration of the compounds was 10 uM or 1 uM for anti-HBV activity assay and 100 uM for cytotoxicity determination. Cell viability was determined by CCK-8 assay. After 6 days of compounds treatment, 20 μl CCK-8 reagents were added to each well of cytotoxicity assay plates. Cell plates were incubated at 37° C., 5% CO2 for 2.5 h. The absorbance at 450 nm wavelength and the absorbance at 630 nm wavelength as reference was measured.

The change of HBV DNA level induced by the compounds was assessed by quantitative real-time polymerase chain reaction (qPCR). Briefly, the HBV DNA in the culture medium was extracted using QlAamp 96 DNA Blood Kit according to the manual and then quantified by real-time PCR assay using the primers and probe in the table 1 below.

TABLE 2

| Primers or | | SEQ ID |
|---|---|---|
| Probe | Sequence | NO: |
| HBV-Fw | GTGTCTGCGGCGTTTTATCA | 1 |
| HBV-Rev | GACAAACGGGCAACATACCTT | 2 |
| HBV-Probe | CCTCTKCATCCTGCTGCTATGCCTCATC | 3 |
| With FAM | | |
| reporter | | |
| and TAMRA | | |
| quencher | | |

2) DATA Analysis

EC₅₀ and CC₅₀ values are calculated by the GRAPHPAD PRISM software. If the CV % of DMSO controls is below 15% and the reference compounds shows expected activity or cytotoxicity, the data of this batch of experiment is considered qualified.

RESULTS: See Table 3 below.

TABLE 3

| | EC₅₀ | CC₅₀ |
|---|---|---|
| Compound ID | (μM) | (μM) |
| I-1A | 0.016 | 59.8 |
| I-2A | 0.033 | 74.0 |
| I-3B | 0.0052 | 60.2 |
| I-4E | 0.0071 | 48.3 |
| I-5 | 0.086 | 40.6 |
| I-6A | 0.018 | 82.4 |
| I-7F | 0.008 | 26.4 |
| I-8F | 0.045 | 37.6 |
| I-9F | 0.11 | 82.6 |
| I-10A | 0.028 | 94.5 |
| I-11A | 0.048 | 97.9 |
| I-12A | 0.31 | >100 |
| I-13A | 0.013 | 67.1 |
| I-14A | 0.033 | >100 |
| I-15A | 0.019 | >100 |
| I-16A | 0.015 | 92.8 |
| I-17B | 0.0026 | 41.7 |
| I-18A | 0.007 | 62.4 |
| I-19A | 0.028 | 59.1 |
| I-20A | 0.001 | 55.0 |
| I-21A | 0.008 | >100 |
| I-22A | 0.0005 | 44.3 |
| I-23A | 0.0005 | 45.0 |
| I-24A | 0.014 | >100 |
| Reference 1A | >1 | 14 |
| Reference 1B | >1 | 2.5 |
| Reference 1C | 0.6774 | 3.7 |
| Reference 1D | 0.8124 | 5.6 |
| Reference 1E | >1 | 11.7 |
| Reference 1F | 0.087 | 5.8 |
| Reference 1G | >1 | 14.5 |
| Reference 1H | >1 | 8.7 |

1. A compound of Formula (I), 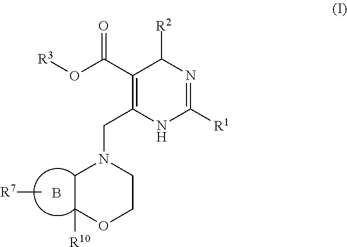
including any possible deuterated isomers, stereoisomers or tautomeric forms thereof, wherein:
R¹ is selected from aryl or heteroaryl, each optionally substituted with one or more substituents selected from halogen, and C₁-C₆alkyl;
R² is selected from aryl or heteroaryl, each substituted with R⁴, R⁵, and R⁶;
R³ is C₁-C₄alkyl;
R⁴, R⁵, and R⁶ independently are selected from the group consisting of H, —OH, —CN, C₁-C₄alkyl, and halogen;
R¹⁰ is selected from the group consisting of H, and C₁-C₄alkyl;
Ring B is selected from the group consisting of 3-8 membered saturated or unsaturated rings, each of the rings optionally comprising 1, 2 or 3 heteroatoms selected from O, N, and S, and each of the rings optionally substituted with one or more substituents independently selected from the group consisting of halogen, ═O, —OH, —CN, C₁-C₄alkyl, C₁-C₄alkyloxy, and hydroxyC₁-C₄alkyl, wherein each C₁-C₄alkyl is optionally substituted with halogen;
R⁷ is selected from the group consisting of —SO₂—R⁸, —SO₂-Q-R⁸, —OC(═O)C₁-C₆alkyl, —C(═O)OC₁-C₆alkyl-COOH, —C(═O)NHC i-C₆alkyl-COOH, —C(═O)O-Q-COOH, —C(═O)NH-Q-COOH, —C(═O)C₁-C₆alkyl, —C(═O)C₁-C₆alkyl-R⁸, —NHC₁-C₆alkyl-R⁸, —C₁-C₆alkyl, —C₁-C₆alkyl-R⁸, —C₁-C₆alkoxyC₁-C₆alkyl-R⁸, —(CH₂)ₚ—R⁸, —(CH₂)ₚ—C(R¹¹R¹²)—R⁸, and —(CH₂)p-Q-R⁸;
R⁸ is selected from the group consisting of —C₁-C₆alkyl, —C₁-C₆alkyl-COOH, —COOH, and carboxylic acid bioisosteres;
R¹¹ and R¹² together with the carbon atom to which they are attached form a 3-8 membered saturated ring optionally substituted with R⁹, the 3-8 membered saturated ring optionally containing a heteroatom, the heteroatom being N or O;
Q is selected from the group consisting of aryl, heteroaryl, and a 3-8 membered saturated ring, each optionally substituted with R⁹, the 3-8 membered saturated ring optionally containing a heteroatom, the heteroatom being N or 0;
R⁹ is selected from the group consisting of H, —C₁-C₆alkyl, —C₁-C₆alkoxyC₁-C₆alkyl and —C₁-C₆alkylcarbonyl;
p is an integer of 0, 1, 2, 3, or 4;
or a pharmaceutically acceptable salt or a solvate thereof.

2. The compound of claim 1, wherein ring B is selected from 5-7 membered saturated rings, each optionally comprising one heteroatom being N, and each optionally substituted with one or more substituents independently selected from the group consisting of halogen, ═O, —OH, —CN, C₁-C₄alkyl, C₁-C₄alkyloxy, and hydroxyC₁-C₄alkyl.

3. The compound of claim 1, which is of the following general formulae:

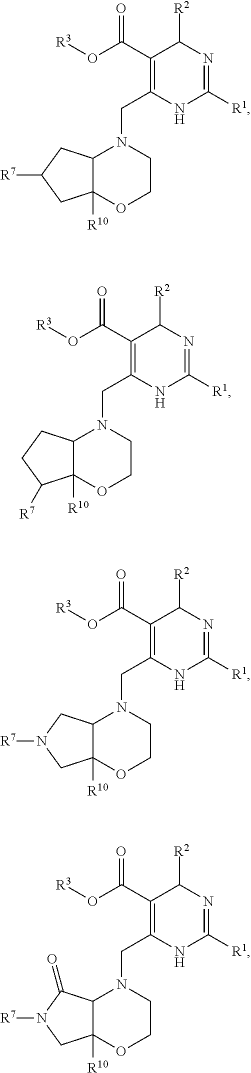

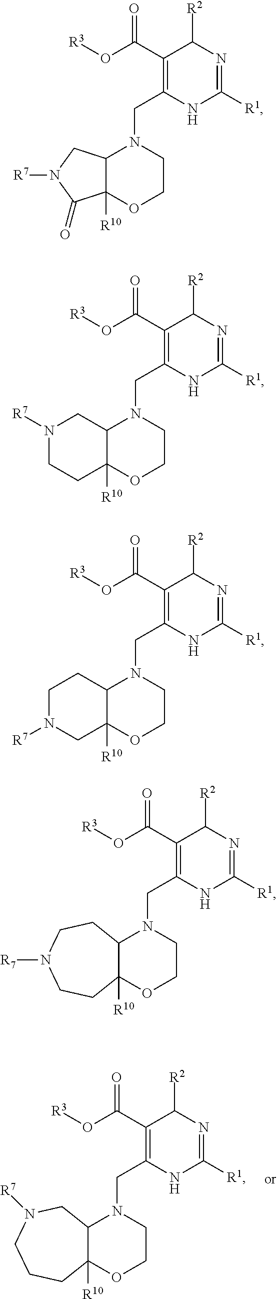

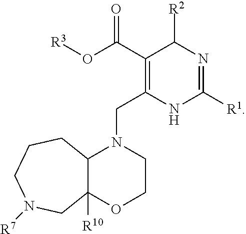

4. The compound of claim 1, wherein R¹ is selected from the group consisting of thiazolyl, pyridyl, and oxazolyl, each optionally substituted with one or more substituents selected from F, and C₁-C₆alkyl.

5. The compound of claim 1, wherein R¹ is thiazolyl.

6. The compound of claim 1, wherein R² is selected from the group consisting of phenyl, thiophenyl, and pyridyl, each substituted with R⁴, R⁵, and R⁶, wherein R⁴, R⁵, and R⁶ independently are selected from the group consisting of H, —OH, —CN, C₁-C₄alkyl, and halogen.

7. The compound of claim 1, wherein R² is phenyl substituted with R⁴, R⁵, and R⁶, wherein R⁴, R⁵, and R⁶ independently are selected from the group consisting of H, —CN, —CH₃, F, Cl and Br.

8. The compound of claim 1, wherein R³ is methyl, ethyl, propyl, or isopropyl.

9. The compound of claim 1, wherein R¹ is selected from the group consisting of —SO₂-Q-R⁸, —C(═O)OC₁-C₆alkyl-COOH, —C(═O)NHC₁-C₆alkyl-COOH, —C(═O)O-Q-COOH, —C(═O)NH-Q-COOH, —C(═O)C₁-C₆alkyl-le, —NHC₁-C₆alkyl-R⁸, —C₁-C₆alkyl-R⁸, —(CH₂)ₚ—R⁸, and —(CH₂)ₚ-Q-R⁸.

10. The compound of claim 1, wherein R⁸ is selected from the group consisting of —C₁-C₆alkyl —COOH, —COOH, —C(═O)NHS(═O)₂C₁-C₆alkyl, and tetrazolyl.

11. The compound of claim 1, wherein Q is selected from the group consisting of aryl, heteroaryl, and a 3-8 membered saturated ring, each optionally substituted with R⁹, the 3-8 membered saturated ring optionally containing a heteroatom, the heteroatom being N or O; and R⁹ is selected from the group consisting of H, —C₁-C₆alkyl.

12. The compound of claim 1, wherein p is an integer of 0, 1, or 2.

13. A compound according to claim 1, selected from the group consisting of the compounds having the following formulae:

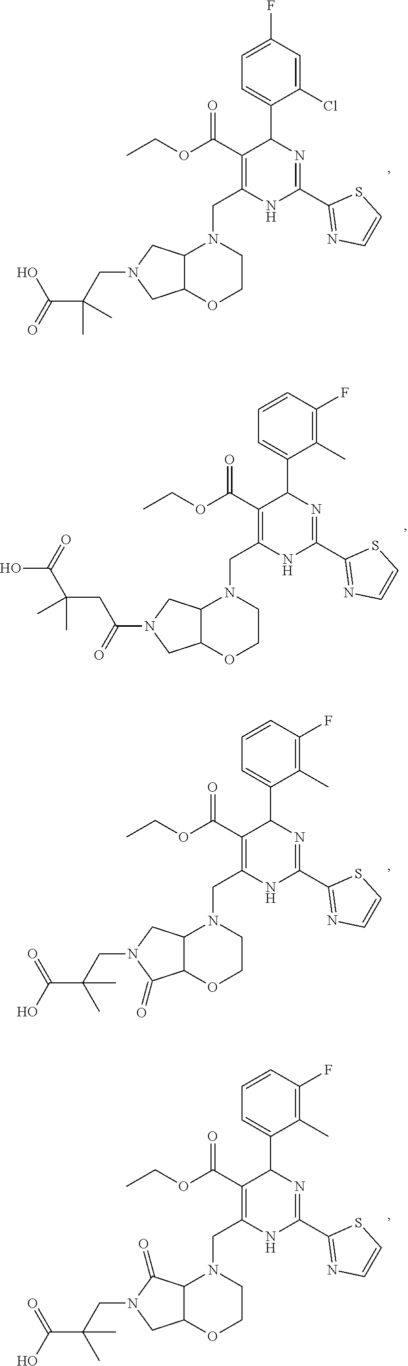 [SMILES: CCOC(=O)C1=C(CN2CCOC3CN(CC(C)(C)C(=O)O)CC32)NC(c2nccs2)=NC1c1ccc(F)cc1Cl ; CCOC(=O)C1=C(CN2CCOC3CN(C(=O)CC(C)(C)C(=O)O)CC32)NC(c2nccs2)=NC1c1cccc(F)c1C ; CCOC(=O)C1=C(CN2CCOC3C(=O)N(CC(C)(C)C(=O)O)CC32)NC(c2nccs2)=NC1c1cccc(F)c1C ; CCOC(=O)C1=C(CN2CCOC3CN(CC(C)(C)C(=O)O)C(=O)C32)NC(c2nccs2)=NC1c1cccc(F)c1C]

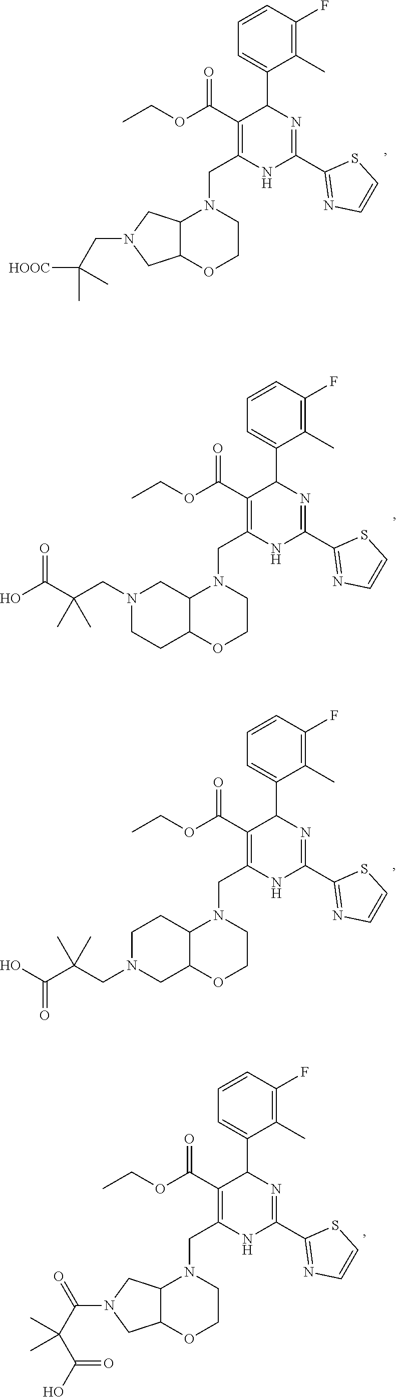 [SMILES: CCOC(=O)C1=C(CN2CCOC3CN(CC(C)(C)C(=O)O)CC32)NC(c2nccs2)=NC1c1cccc(F)c1C ; CCOC(=O)C1=C(CN2CCOC3CCN(CC(C)(C)C(=O)O)CC32)NC(c2nccs2)=NC1c1cccc(F)c1C ; CCOC(=O)C1=C(CN2CCOC3CN(CC(C)(C)C(=O)O)CCC32)NC(c2nccs2)=NC1c1cccc(F)c1C ; CCOC(=O)C1=C(CN2CCOC3CN(C(=O)C(C)(C)C(=O)O)CC32)NC(c2nccs2)=NC1c1cccc(F)c1C]

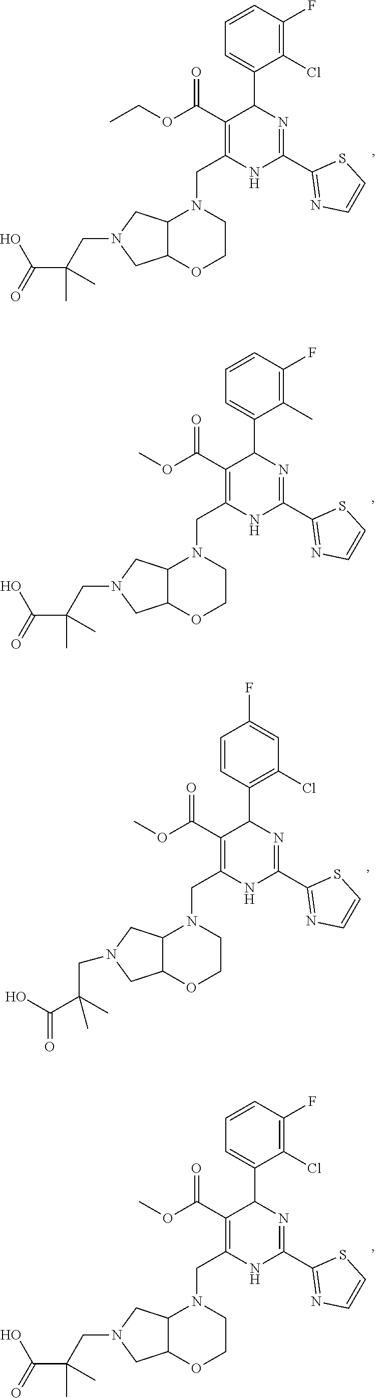 [SMILES: CCOC(=O)C1=C(CN2CCOC3CN(CC(C)(C)C(=O)O)CC32)NC(c2nccs2)=NC1c1cccc(F)c1Cl ; COC(=O)C1=C(CN2CCOC3CN(CC(C)(C)C(=O)O)CC32)NC(c2nccs2)=NC1c1cccc(F)c1C ; COC(=O)C1=C(CN2CCOC3CN(CC(C)(C)C(=O)O)CC32)NC(c2nccs2)=NC1c1ccc(F)cc1Cl ; COC(=O)C1=C(CN2CCOC3CN(CC(C)(C)C(=O)O)CC32)NC(c2nccs2)=NC1c1cccc(F)c1Cl]

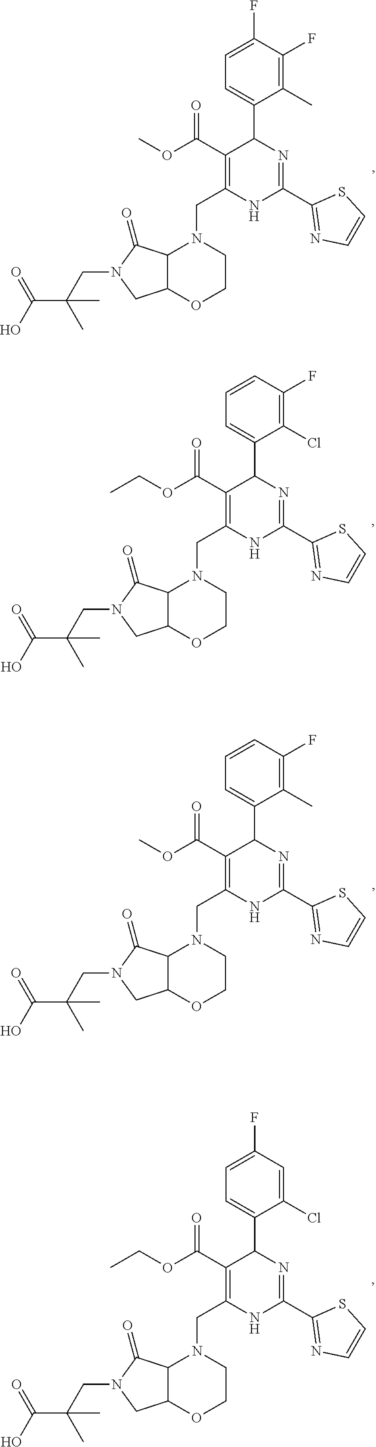 [SMILES: COC(=O)C1=C(CN2CCOC3CN(CC(C)(C)C(=O)O)C(=O)C32)NC(c2nccs2)=NC1c1ccc(F)c(F)c1C ; CCOC(=O)C1=C(CN2CCOC3CN(CC(C)(C)C(=O)O)C(=O)C32)NC(c2nccs2)=NC1c1cccc(F)c1Cl ; COC(=O)C1=C(CN2CCOC3CN(CC(C)(C)C(=O)O)C(=O)C32)NC(c2nccs2)=NC1c1cccc(F)c1C ; CCOC(=O)C1=C(CN2CCOC3CN(CC(C)(C)C(=O)O)C(=O)C32)NC(c2nccs2)=NC1c1ccc(F)cc1Cl]

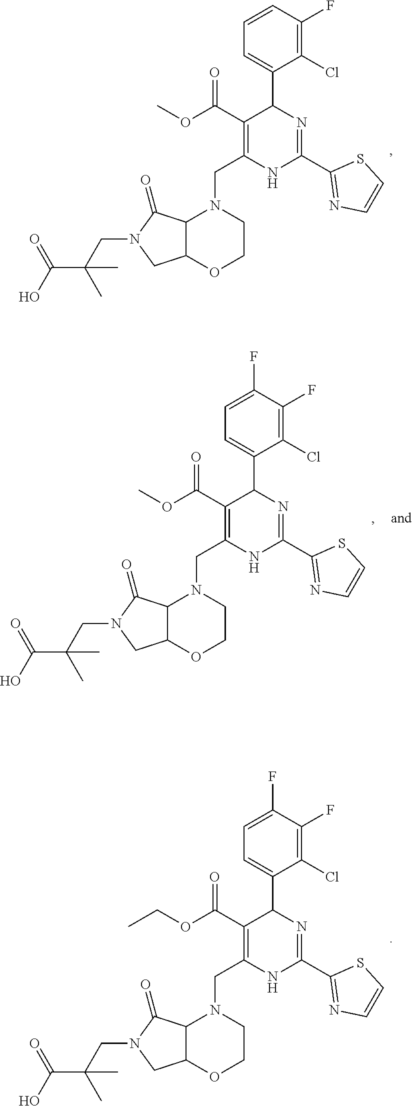 [SMILES: COC(=O)C1=C(CN2CCOC3CN(CC(C)(C)C(=O)O)C(=O)C32)NC(c2nccs2)=NC1c1cccc(F)c1Cl ; COC(=O)C1=C(CN2CCOC3CN(CC(C)(C)C(=O)O)C(=O)C32)NC(c2nccs2)=NC1c1ccc(F)c(F)c1Cl ; CCOC(=O)C1=C(CN2CCOC3CN(CC(C)(C)C(=O)O)C(=O)C32)NC(c2nccs2)=NC1c1ccc(F)c(F)c1Cl]

14. A pharmaceutical composition, which comprises the compound of claim 1 and at least one pharmaceutically acceptable carrier.

15. (canceled)

16. A method of preventing or treating an HBV infection or an HBV-induced disease in a mammal in need thereof, the method comprising administering to the mammal an effective amount of the pharmaceutical composition of claim 14.

17. A method of preventing or treating chronic Hepatitis B in a subject in need thereof, the method comprising administering to the subject an effective amount of the pharmaceutical composition of claim 14.

18. A product comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the compound or pharmaceutically acceptable salt of claim 1, and wherein said second compound is another HBV inhibitor which is selected from the group consisting of HBV combination drugs, HBV DNA polymerase inhibitors, immunomodulators, toll-like (TLR) receptor modulators, interferon alpha receptor ligands, hyaluronidase inhibitors, hepatitis b surface antigen (HbsAg) inhibitors, cytotoxic T-lymphocyte-associated protein 4 (ipi4) inhibitors, cyclohilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, farnsoid X receptor agonists, HBV antibodies, CCR2 chemokine antagonists, thymosin agonists, cytokines, nuceloprotein modulators, retinoic acid-inducible gene 1 stimulators, NOD2 stimulators, phosphatidylinositol 3-kinase (P13K) inhibitors, indoleamine 2,3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and anti-HBV drugs.

19. A process for the preparation of a compound according to claim 1, comprising the steps of:
a. condensing of aldehyde of Formula (II), wherein Formula (II) is 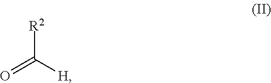
acetoacetate of Formula (III), wherein Formula (III) is 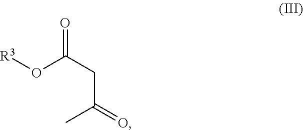
and amidine of Formula (IV), wherein Formula (IV) is 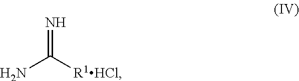
in the presence of a base, the base being preferably to form a compound according to Formula (I-1): 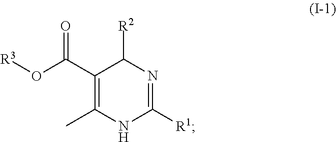
b. reacting the compound of Formula (I-1) with a brominating agent to form a compound according to Formula (I-2), wherein Formula (I-2) is 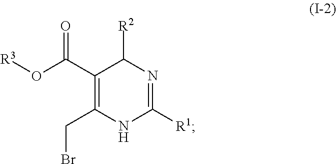
and
c. coupling the compound of Formula (I-2) with a compound of Formula (V), wherein Formula (V) is 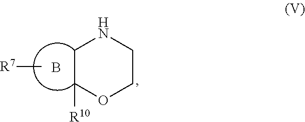
in the presence of a base, to form the compound according to Formula (I).

20. The process of claim 19, wherein the base in step a is NaOAc.

21. The process of claim 19, wherein the brominating agent in step b is N-bromosuccinimide.

22. The process of claim 19, wherein the base in step c is triethyanolamine.


SEQUENCE LISTING (3 sequences)

SEQ ID NO: 1 - DNA, 20 bp, Artificial
  primer
gtgtctgcggcgttttatca

SEQ ID NO: 2 - DNA, 21 bp, Artificial
  primer
gacaaacgggcaacatacctt

SEQ ID NO: 3 - DNA, 28 bp, Artificial
  probe
cctctkcatcctgctgctatgcctcatc